(19) United States
(12) Patent Application Publication
(10) Pub. No.: US 2017/0136085 A1
(43) Pub. Date: May 18, 2017

(54) TARGETED THERAPEUTICS

(71) Applicant: SYNTA PHARMACEUTICALS CORP., LEXINGTON, MA (US)

(72) Inventors: Shoujun Chen, Bedford, MA (US); Genliang Lu, Winchester, MA (US); Sami Osman, Cambridge, MA (US)

(21) Appl. No.: 15/314,769

(22) PCT Filed: May 29, 2015
(86) PCT No.: PCT/US15/33154

§ 371 Date: Nov. 29, 2016

Related U.S. Application Data
(60) Provisional application No. 62/004,519, filed on May 29, 2014.

(51) Int. Cl. A61K 38/07

(52) U.S. Cl. CPC A61K 38/07; A61K 47/48061

(57) ABSTRACT

The present invention provides pharmacological compounds including a peptide epoxy ketone protease inhibitor conjugated to a binding moiety that directs the peptide epoxy ketone protease inhibitor to a biological target of interest. Likewise, the present invention provides compositions, kits, and methods (e.g., therapeutic, diagnostic, and imaging) including the compounds. The compounds can be described as a protein interacting binding moiety-drug conjugate (SDC-TRAP) compounds, which include a protein interacting binding moiety and a peptide epoxy ketone protease inhibitor. For example, in certain embodiments directed to treating cancer, the SDC-TRAP can include an Hsp90 inhibitor conjugated to a cytotoxic agent as the peptide epoxy ketone protease inhibitor.

### RELATED APPLICATIONS

This application claims the benefit of priority to U.S. Provisional Patent Application No. 62/004,519, filed on May 29, 2014, the entire contents of which are hereby incorporated herein by reference.

### FIELD OF THE INVENTION

Pharmacological compounds including a peptide epoxy ketone protease inhibitor conjugated to a binding moiety that directs the protease inhibitor to a biological target of interest are disclosed herein. Such compounds can specifically direct the protease inhibitor to target cells or tissue of interest, for targeted chemotherapeutic treatment of conditions such as cancer, or for treating inflammatory conditions.

### BACKGROUND OF THE INVENTION

The proteasome has been validated as a therapeutic target, as demonstrated by the FDA approval of bortezomib (i.e., VELCADE) for the treatment of various cancer indications, including multiple myeloma. More highly proteasome-specific inhibitors that could have fewer toxic side effects have also been described, including peptide epoxy ketones such as epoxomicin, described in U.S. Pat. No. 6,831,099, and those described in U.S. Pat. Nos. 7,687,456; 7,737,112; 7,232,818; 7,417,042; 8,080,576; 8,088,741; and 8,357,683. While such compounds have promise, improvements in administration, e.g., enhanced solubility, permeability, pharmacokinetics and/or pharmacodynamics compared with the corresponding epoxy ketone protease inhibitors that do not contain such moieties continue to be sought. In some cases it would be advantageous to be able to administer such compounds in a form wherein they exhibit lower therapeutic activity when compared with said corresponding epoxy ketone protease inhibitors, but are rendered fully active once reaching the compound target.

One approach at providing such improvements can be found, e.g., in WO2014/011695 (“'695”), which describes prodrugs of certain peptide epoxy ketone proteasome inhibitors. In some embodiments, the epoxy moiety of the protease inhibitor is replaced with an activated diol, which can contain a PEG moiety. In some embodiments, the ketone moiety of the epoxy ketone protease inhibitors is replaced with a masked ketone moiety, e.g., acylhydrazones, oximes, oxazolidine, or thiazolidine; the masked ketone moiety can optionally contain a PEG moiety. In some embodiments, these proteasome inhibitor prodrugs are cleavable by pH change and/or enzymes such as esterases, cytochrome P450, phosphodiesterase, phosphoamidase, phosphatase, and DT-diaphorase. The '695 publication posits that temporarily masking the protease inhibitory activity until the compounds have reached and released the active into system circulation can reduce side effects associated with routes of administration, also facilitating subcutaneous administration and extending half-life.

However, often times even with the most promising drugs, the applicability and/or effectiveness of chemotherapy, as well as other therapies and diagnostics employing potentially toxic moieties, is limited by undesired side effects. While the '695 publication does appear to address this by providing means for modifying peptide epoxy ketone protease inhibitors, it provides little or no guidance on enabling enhanced target specificity, focusing instead on describing PEGylated protease inhibitor compounds.

Many disease and disorders are characterized by the presence of high levels of certain proteins in specific types of cells. In some cases, the presence of these high levels of protein is caused by overexpression. Historically, some of these proteins have been useful targets for therapeutic molecules or used as biomarkers for the detection of disease. One class of overexpressed intracellular protein that has been recognized as a useful therapeutic target is known as the heat shock proteins.

Heat shock proteins (HSPs) are a class of proteins that are up-regulated in response to elevated temperature and other environmental stresses, such as ultraviolet light, nutrient deprivation, and oxygen deprivation. HSPs have many known functions, including acting as chaperones to other cellular proteins (called client proteins) to facilitate their proper folding and repair, and to aid in the refolding of misfolded client proteins. There are several known families of HSPs, each having its own set of client proteins. Hsp90 is one of the most abundant HSP families, accounting for about 1-2% of proteins in a cell that is not under stress and increasing to about 4-6% in a cell under stress.

Inhibition of Hsp90 results in degradation of its client proteins via the ubiquitin proteasome pathway. Unlike other chaperone proteins, the client proteins of Hsp90 are mostly protein kinases or transcription factors involved in signal transduction, and a number of its client proteins have been shown to be involved in the progression of cancer. Hsp90 has been shown by mutational analysis to be necessary for the survival of normal eukaryotic cells, but Hsp90 is overexpressed in many tumor types, indicating that it may play a significant role in the survival of cancer cells and that cancer cells may be more sensitive to inhibition of Hsp90 than normal cells. For example, cancer cells typically have a large number of mutated and overexpressed oncoproteins that are dependent on Hsp90 for folding. In addition, because the environment of a tumor is typically hostile due to hypoxia, nutrient deprivation, acidosis, etc., tumor cells may be especially dependent on Hsp90 for survival. In view of the above, Hsp90 has been an attractive target of drug development.

### SUMMARY OF THE INVENTION

Pharmacological molecules (“SDC-TRAPs”) including a peptide epoxy ketone protease inhibitor conjugated to a binding moiety, which directs the peptide epoxy ketone protease inhibitor into a target cell of interest in a manner that traps the molecule in the target cell, are disclosed herein. In one embodiment, the peptide epoxy ketone protease inhibitor moiety is conjugated via a cleavable bond or linker to the binding moiety, such that the peptide epoxy ketone protease inhibitor bond or linker is preferentially cleaved after the SDC-TRAP enters the target cell. As described in detail in, e.g., WO2013158644, the properties of SDC-TRAP molecules can be used to selectively deliver a peptide epoxy ketone protease inhibitor moiety to a specific type of cell in order to increase the intracellular level of the peptide epoxy ketone protease inhibitor moiety in the target cell as compared to other cells. SDC-TRAP molecules enter target cells by passive diffusion and are selectively retained in the target cells, and are selectively retained only in cells that overexpress or otherwise have a high intracellular level of the protein to which the binding moiety binds.

The present invention provides compositions, kits, and methods (e.g., therapeutic, diagnostic, and imaging) including the compounds.

Accordingly, in one aspect, the present invention features a binding moiety-drug conjugate (SDC-TRAP) comprising a binding moiety and an effector moiety.

In another aspect, the present invention features a SDC-TRAP comprising a binding moiety and an effector moiety, wherein the SDC-TRAP is able to enter a cell by passive diffusion or active transport.

In one embodiment, the effector moiety is a therapeutic moiety. In a further embodiment, the therapeutic moiety is a cytotoxic moiety. In another further embodiment, the therapeutic moiety is a peptide epoxy ketone protease inhibitor.

In another aspect, the present invention features a SDC-TRAP comprising an Hsp90 binding moiety and a peptide epoxy ketone protease inhibitor.

In one embodiment, the SDC-TRAP is selected from SDC-TRAP-1001, SDC-TRAP-1002, SDC-TRAP-1003, SDC-TRAP-1004, SDC-TRAP-1005, SDC-TRAP-1006, SDC-TRAP-1007, SDC-TRAP-1008, SDC-TRAP-1009, SDC-TRAP-1010, SDC-TRAP-1011, SDC-TRAP-1012, SDC-TRAP-1013, SDC-TRAP-1014, SDC-TRAP-1015, SDC-TRAP-1016, SDC-TRAP-1017, SDC-TRAP-1018, SDC-TRAP-1019, SDC-TRAP-1020, SDC-TRAP-1021, SDC-TRAP-1022, SDC-TRAP-1023, SDC-TRAP-1024, SDC-TRAP-1025, SDC-TRAP-1026, SDC-TRAP-10127, SDC-TRAP-1028, SDC-TRAP-1029, SDC-TRAP-1030, SDC-TRAP-1031, SDC-TRAP-1032, SDC-TRAP-1033 and SDC-TRAP-1034.

In one embodiment, the binding moiety and the effector moiety are covalently attached. In another embodiment, the binding moiety and the effector moiety are covalently attached by a linker. In a further embodiment, the linker comprises a cleavable linker. In another further embodiment, the cleavable linker comprises an enzymatically cleavable linker. In another embodiment, the linker is selected from disulfide, carbamate, amide, ester, and ether linkers.

The SDC-TRAP/peptide epoxy ketone protease inhibitor conjugates provide numerous advantages. For example, SDC-TRAP/peptide epoxy ketone protease inhibitor conjugates can provide for targeted therapy, maximizing efficacy and/or minimizing undesired side effects. The SDC-TRAP can also provide for targeted use of a peptide epoxy ketone protease inhibitor that would otherwise be unsuitable for administration alone due to toxicity and/or undesired systemic effects. Alternatively, the SDC-TRAP can deliver its peptide epoxy ketone protease inhibitor payload in a selective manner a cytotoxic molecule to destroy a target cell, such as a cancer or inflammatory cell.

In various aspects and embodiments, the SDC-TRAP can exhibit decreased and/or minimized toxicity concurrently with increased efficacy (e.g., as compared to that of the peptide epoxy ketone protease inhibitor when used alone). Decreasing and/or minimizing toxicity can encompass reducing toxicity to a predetermined level (e.g., a regulatory guideline or suggested level such as promulgated by the US Food and Drug Administration (“FDA”)). Increasing efficacy can encompass increasing efficacy to a predetermined level (e.g., a regulatory guideline or suggested level). Similarly, decreasing and/or minimizing toxicity concurrently with increasing efficacy can encompass achieving a predetermined therapeutic ratio (e.g., a regulatory guideline or suggested value).

Accordingly, in one embodiment, the SDC-TRAP exhibits decreased toxicity and increased efficacy compared to the effector moiety or the binding moiety used alone.

Decreasing and/or minimizing toxicity can encompass, for example, reducing toxicity by 5, 10, 15, 20, 25, 30, 35, 40, 45, 50, 55, 60, 65, 70, 75, 80, 85, 90, 95%, or more. Increasing efficacy can encompass, for example, increasing efficacy by 5, 10, 15, 20, 25, 30, 35, 40, 45, 50, 55, 60, 65, 70, 75, 80, 85, 90, 95, 100, 125, 150, 175, 200, 250, 300, 400, 500%, or more. Decreasing and/or minimizing toxicity concurrently with increasing efficacy can encompass, for example: essentially the same efficacy with decreased toxicity; essentially the same toxicity with increased efficacy; or decreased toxicity and increased efficacy. Similarly, decreasing and/or minimizing toxicity concurrently with increasing efficacy can encompass, for example, scenarios such as: increased efficacy enabling a lower dose (e.g., lower dose of peptide epoxy ketone protease inhibitor with a correspondingly lower net toxicity) and decreased toxicity enabling a higher dose (e.g., higher dose of peptide epoxy ketone protease inhibitor without a correspondingly higher net toxicity).

Additional advantages are discussed in detail below. While many of the illustrative embodiments and examples are presented in the context of cancer, a person of ordinary skill in the art would understand that the present invention has applications across therapeutic, diagnostic, and imaging applications that require, or would benefit from, targeting of a peptide epoxy ketone protease inhibitor.

In various aspects, the invention provides an SDC-TRAP comprising a binding moiety (e.g., Hsp90 binding moiety) and a peptide epoxy ketone protease inhibitor, wherein the SDC-TRAP is present in target (e.g., cancer) cells for at least 24 hours. In one embodiment, the SDC-TRAP is present in a target cell for at least 24 hours.

In various aspects, the invention provides an SDC-TRAP comprising a binding moiety (e.g., Hsp90 binding moiety) and a peptide epoxy ketone protease inhibitor, wherein the peptide epoxy ketone protease inhibitor is released for a period of at least 6 hours (e.g., within a target cell and/or tissue). In another embodiment, the SDC-TRAP is released in a target cell for at least 6 hours.

In various aspects, the invention provides an SDC-TRAP comprising a binding moiety (e.g., Hsp90 binding moiety) and a peptide epoxy ketone protease inhibitor, wherein the peptide epoxy ketone protease inhibitor is selectively released inside a target (e.g., cancer) cell.

In various aspects, the invention provides an SDC-TRAP comprising a binding moiety (e.g., Hsp90 binding moiety) and a peptide epoxy ketone protease inhibitor, wherein the Hsp90 is an inhibitor (e.g., Hsp90 inhibitor) that is ineffective as a therapeutic agent when administered alone.

In various aspects, the invention provides pharmaceutical compositions comprising a therapeutically effective amount of at least one SDC-TRAP, and at least one pharmaceutical excipient.

In one embodiment, the SDC-TRAP comprises the SDC-TRAP of any of the aspects described herein.

In another aspect, the present invention provides pharmaceutical compositions comprising a therapeutically effective amount of at least one SDC-TRAP comprising an Hsp90 binding moiety and a peptide epoxy ketone protease inhibitor, and at least one pharmaceutical excipient.

In one embodiment, the SDC-TRAP is selected from SDC-TRAP-1001, SDC-TRAP-1002, SDC-TRAP-1003, SDC-TRAP-1004, SDC-TRAP-1005, SDC-TRAP-1006, SDC-TRAP-1007, SDC-TRAP-1008, SDC-TRAP-1009, SDC-TRAP-1010, SDC-TRAP-1011, SDC-TRAP-1012, SDC-TRAP-1013, SDC-TRAP-1014, SDC-TRAP-1015, SDC-TRAP-1016, SDC-TRAP-1017, SDC-TRAP-1018, SDC-TRAP-1019, SDC-TRAP-1020, SDC-TRAP-1021, SDC-TRAP-1022, SDC-TRAP-1023, SDC-TRAP-1024, SDC-TRAP-1025, SDC-TRAP-1026, SDC-TRAP-10127, SDC-TRAP-1028, SDC-TRAP-1029, SDC-TRAP-1030, SDC-TRAP-1031, SDC-TRAP-1032, SDC-TRAP-1033 and SDC-TRAP-1034.

In one aspect, the invention provides methods for treating a subject in need thereof comprising administering a therapeutically effective amount of at least one SDC-TRAP to the subject, thereby treating the subject.

In another aspect, the invention provides methods for treating a subject in need thereof comprising administering a therapeutically effective amount of at least one SDC-TRAP comprising an Hsp90 binding moiety and a peptide epoxy ketone protease inhibitor to the subject, thereby treating the subject.

In one embodiment, the subject is suffering from a disease of disorder selected from cancer, autoimmune disease, graft or transplant-related condition, neurodegenerative disease, fibrotic-associated condition, ischemic-related conditions, infection (viral, parasitic or prokaryotic) and diseases associated with bone loss. In a particular embodiment, the subject is suffering from cancer.

In one aspect, the invention provides methods for imaging, diagnosing, and/or selecting a subject comprising administering an effective amount of at least one SDC-TRAP to the subject, thereby imaging, diagnosing, and/or selecting the subject.

In one embodiment of the above aspects, the SDC-TRAP is selected from SDC-TRAP-1001, SDC-TRAP-1002, SDC-TRAP-1003, SDC-TRAP-1004, SDC-TRAP-1005, SDC-TRAP-1006, SDC-TRAP-1007, SDC-TRAP-1008, SDC-TRAP-1009, SDC-TRAP-1010, SDC-TRAP-1011, SDC-TRAP-1012, SDC-TRAP-1013, SDC-TRAP-1014, SDC-TRAP-1015, SDC-TRAP-1016, SDC-TRAP-1017, SDC-TRAP-1018, SDC-TRAP-1019, SDC-TRAP-1020, SDC-TRAP-1021, SDC-TRAP-1022, SDC-TRAP-1023, SDC-TRAP-1024, SDC-TRAP-1025, SDC-TRAP-1026, SDC-TRAP-10127, SDC-TRAP-1028, SDC-TRAP-1029, SDC-TRAP-1030, SDC-TRAP-1031, SDC-TRAP-1032, SDC-TRAP-1033 and SDC-TRAP-1034.

In various aspects, the invention provides kits for treating a subject in need thereof comprising at least one SDC-TRAP and instruction for administering a therapeutically effective amount of the at least one SDC-TRAP to the subject, thereby treating the subject.

In various aspects, the invention provides kits for imaging, diagnosing, and/or selecting a subject comprising at least one SDC-TRAP and instruction for administering an effective amount of at least one SDC-TRAP to the subject, thereby imaging, diagnosing, and/or selecting the subject.

In various embodiments, the invention can include any one or more of the aspects disclosed herein having any one or more of the features disclosed herein.

In one embodiment, the binding moiety interacts with a protein that is overexpressed in a target cell compared to a normal cell. In a further embodiment, the target cell is a cancer cell.

In another embodiment, the protein is a chaperonin protein. In a related embodiment, the chaperonin is Hsp90.

In another embodiment, the chaperonin is an Hsp90 binding moiety.

In another further embodiment, the binding moiety is an Hsp90 ligand or a prodrug thereof. In one embodiment, the Hsp90 ligand is an Hsp90 inhibitor. In a further embodiment, the Hsp90 inhibitor is selected from ganetespib, geldanamycins, macbecins, tripterins, tanespimycins, and radicicols.

In various embodiments, the binding moiety can be an Hsp90-targeting moiety, for example a triazole/resorcinol-based compound that binds Hsp90, or a resorcinol amide-based compound that binds Hsp90, e.g., ganetespib, AUY-922, or AT-13387.

In various embodiments, the binding moiety can be an Hsp90-binding compound of formula (I):

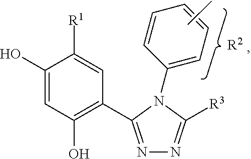

wherein

R¹ may be alkyl, aryl, halide, carboxamide or sulfonamide; R² may be alkyl, cycloalkyl, aryl or heteroaryl, wherein when R² is a six-membered aryl or heteroaryl, R² is substituted at the 3- and 4-positions relative to the connection point on the triazole ring, through which a linker L is attached; and R³ may be SH, OH, —CONHR⁴, aryl or heteroaryl, wherein when R³ is a six-membered aryl or heteroaryl, R³ is substituted at the 3 or 4 position.

In various embodiments, the binding moiety can be an Hsp90-binding compound of formula (II):

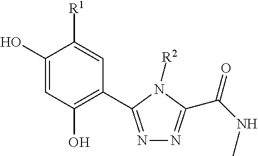

wherein

R¹ may be alkyl, aryl, halo, carboxamido, sulfonamido; and R² may be optionally substituted alkyl, cycloalkyl, aryl or heteroaryl. Examples of such compounds include 5-(2,4-dihydroxy-5-isopropylphenyl)-N-(2-morpholinoethyl)-4-(4-(morpholinomethyl)phenyl)-4H-1,2,4-triazole-3-carboxamide and 5-(2,4-dihydroxy-5-isopropylphenyl)-4-(4-(4-methylpiperazin-1-yl)phenyl)-N-(2,2,2-trifluoroethyl)-4H-1,2,4-triazole-3-carboxamide.

In various embodiments, the binding moiety can be an Hsp90-binding compound of formula (III):

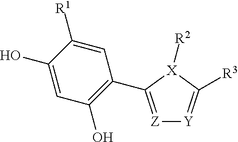

wherein

X, Y, and Z may independently be CH, N, O or S (with appropriate substitutions and satisfying the valency of the corresponding atoms and aromaticity of the ring); R¹ may be alkyl, aryl, halide, carboxamido or sulfonamido; R² may be substituted alkyl, cycloalkyl, aryl or heteroaryl, where a linker L is connected directly or to the extended substitutions on these rings; R³ may be SH, OH, NR⁴R⁵ AND —CONHR⁶, to which a peptide epoxy ketone protease inhibitor may be connected; R⁴ and R⁵ may independently be H, alkyl, aryl, or heteroaryl; and R⁶ may be alkyl, aryl, or heteroaryl, having a minimum of one functional group to which a peptide epoxy ketone protease inhibitor may be connected.

As used herein, the term “alkyl” means a saturated straight chain or branched non-cyclic hydrocarbon having from 1 to 10 carbon atoms. Representative saturated straight chain alkyls include methyl, ethyl, n-propyl, n-butyl, n-pentyl, n-hexyl, n-heptyl, n-octyl, n-nonyl and n-decyl; while saturated branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like. The term “(C₁-C₆)alkyl” means a saturated straight chain or branched non-cyclic hydrocarbon having from 1 to 6 carbon atoms. Representative (C₁-C₆)alkyl groups are those shown above having from 1 to 6 carbon atoms. Alkyl groups included in compounds of this invention may be optionally substituted with one or more substituents.

As used herein, the term “alkenyl” means a saturated straight chain or branched non-cyclic hydrocarbon having from 2 to 10 carbon atoms and having at least one carbon-carbon double bond. Representative straight chain and branched (C₂-C₁₀)alkenyls include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl, 3-decenyl and the like. Alkenyl groups may be optionally substituted with one or more substituents.

As used herein, the term “alkynyl” means a saturated straight chain or branched non-cyclic hydrocarbon having from 2 to 10 carbon atoms and having at least one carbon-carbon triple bond. Representative straight chain and branched alkynyls include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl, and the like. Alkynyl groups may be optionally substituted with one or more substituents.

As used herein, the term “cycloalkyl” means a saturated, mono- or polycyclic alkyl radical having from 3 to 20 carbon atoms. Representative cycloalkyls include cyclopropyl, 1-methylcyclopropyl, cyclobutyl, cyclopentyl, cyclohexyl, cycloheptyl, cyclooctyl, cyclononyl, -cyclodecyl, octahydro-pentalenyl, and the like. Cycloalkyl groups may be optionally substituted with one or more substituents.

As used herein, the term “cycloalkenyl” means a mono- or poly-cyclic non-aromatic alkyl radical having at least one carbon-carbon double bond in the cyclic system and from 3 to 20 carbon atoms. Representative cycloalkenyls include cyclopentenyl, cyclopentadienyl, cyclohexenyl, cyclohexadienyl, cycloheptenyl, cycloheptadienyl, cycloheptatrienyl, cyclooctenyl, cyclooctadienyl, cyclooctatrienyl, cyclooctatetraenyl, cyclononenyl, cyclononadienyl, cyclodecenyl, cyclodecadienyl, 1,2,3,4,5,8-hexahydronaphthalenyl and the like. Cycloalkenyl groups may be optionally substituted with one or more substituents.

As used herein, the term “haloalkyl” means and alkyl group in which one or more (including all) the hydrogen radicals are replaced by a halo group, wherein each halo group is independently selected from —F, —Cl, —Br, and —I. The term “halomethyl” means a methyl in which one to three hydrogen radical(s) have been replaced by a halo group. Representative haloalkyl groups include trifluoromethyl, bromomethyl, 1,2-dichloroethyl, 4-iodobutyl, 2-fluoropentyl, and the like.

As used herein, an “alkoxy” is an alkyl group which is attached to another moiety via an oxygen linker.

As used herein, a “haloalkoxy” is an haloalkyl group which is attached to another moiety via an oxygen linker.

As used herein, the term an “aromatic ring” or “aryl” means a hydrocarbon monocyclic or polycyclic radical in which at least one ring is aromatic. Examples of suitable aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. Aryl groups may be optionally substituted with one or more substituents. In one embodiment, the aryl group is a monocyclic ring, wherein the ring comprises 6 carbon atoms, referred to herein as “(C₆)aryl.”

As used herein, the term “aralkyl” means an aryl group that is attached to another group by a (C₁-C₆)alkylene group. Representative aralkyl groups include benzyl, 2-phenyl-ethyl, naphth-3-yl-methyl and the like. Aralkyl groups may be optionally substituted with one or more substituents.

As used herein, the term “alkylene” refers to an alkyl group that has two points of attachment. The term “(C₁-C₆)alkylene” refers to an alkylene group that has from one to six carbon atoms. Straight chain (C₁-C₆)alkylene groups are preferred. Non-limiting examples of alkylene groups include methylene (—CH₂—), ethylene (—CH₂CH₂—), n-propylene (—CH₂CH₂CH₂—), isopropylene (—CH₂CH(CH₃)—), and the like. Alkylene groups may be optionally substituted with one or more substituents.

As used herein, the term “heterocyclyl” means a monocyclic (typically having 3- to 10-members) or a polycyclic (typically having 7- to 20-members) heterocyclic ring system which is either a saturated ring or a unsaturated non-aromatic ring. A 3- to 10-membered heterocycle can contain up to 5 heteroatoms; and a 7- to 20-membered heterocycle can contain up to 7 heteroatoms. Typically, a heterocycle has at least on carbon atom ring member. Each heteroatom is independently selected from nitrogen, which can be oxidized (e.g., N(O)) or quaternized; oxygen; and sulfur, including sulfoxide and sulfone. The heterocycle may be attached via any heteroatom or carbon atom. Representative heterocycles include morpholinyl, thiomorpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyrindinyl, tetrahydropyrimidinyl, tetrahydrothiophenyl, tetrahydrothiopyranyl, and the like. A heteroatom may be substituted with a protecting group known to those of ordinary skill in the art, for example, the hydrogen on a nitrogen may be substituted with a tert-butoxycarbonyl group. Furthermore, the heterocyclyl may be optionally substituted with one or more substituents. Only stable isomers of such substituted heterocyclic groups are contemplated in this definition.

As used herein, the term “heteroaromatic”, “heteroaryl” or like terms means a monocyclic or polycyclic heteroaromatic ring comprising carbon atom ring members and one or more heteroatom ring members. Each heteroatom is independently selected from nitrogen, which can be oxidized (e.g., N(O)) or quaternized; oxygen; and sulfur, including sulfoxide and sulfone. Representative heteroaryl groups include pyridyl, 1-oxo-pyridyl, furanyl, benzo[1,3]dioxolyl, benzo[1,4]dioxinyl, thienyl, pyrrolyl, oxazolyl, imidazolyl, thiazolyl, a isoxazolyl, quinolinyl, pyrazolyl, isothiazolyl, pyridazinyl, pyrimidinyl, pyrazinyl, a triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, imidazo[1,2-a]pyridyl, and benzothienyl. In one embodiment, the heteroaromatic ring is selected from 5-8 membered monocyclic heteroaryl rings. The point of attachment of a heteroaromatic or heteroaryl ring to another group may be at either a carbon atom or a heteroatom of the heteroaromatic or heteroaryl rings. Heteroaryl groups may be optionally substituted with one or more substituents.

As used herein, the term “(C₅)heteroaryl” means an aromatic heterocyclic ring of 5 members, wherein at least one carbon atom of the ring is replaced with a heteroatom such as, for example, oxygen, sulfur or nitrogen. Representative (C₅)heteroaryls include furanyl, thienyl, pyrrolyl, oxazolyl, imidazolyl, thiazolyl, isoxazolyl, pyrazolyl, isothiazolyl, pyrazinyl, triazolyl, thiadiazolyl, and the like.

As used herein, the term “(C₆)heteroaryl” means an aromatic heterocyclic ring of 6 members, wherein at least one carbon atom of the ring is replaced with a heteroatom such as, for example, oxygen, nitrogen or sulfur. Representative (C₆)heteroaryls include pyridyl, pyridazinyl, pyrazinyl, triazinyl, tetrazinyl and the like.

As used herein, the term “heteroaralkyl” means a heteroaryl group that is attached to another group by a (C₁-C₆)alkylene. Representative heteroaralkyls include 2-(pyridin-4-yl)-propyl, 2-(thien-3-yl)-ethyl, imidazol-4-yl-methyl and the like. Heteroaralkyl groups may be optionally substituted with one or more substituents.

As used herein, the term “halogen” or “halo” means —F, —Cl, —Br or —I.

Suitable substituents for an alkyl, alkylene, alkenyl, alkynyl, cycloalkyl, cycloalkenyl, heterocyclyl, aryl, aralkyl, heteroaryl, and heteroaralkyl groups include any substituent which will form a stable compound of the invention. Examples of substituents for an alkyl, alkylene, alkenyl, alkynyl, cycloalkyl, cycloalkenyl, heterocyclyl, aryl, aralkyl, heteroaryl, and heteroarylalkyl include an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, or a haloalkyl.

In addition, alkyl, cycloalkyl, alkylene, a heterocyclyl, and any saturated portion of a alkenyl, cycloalkenyl, alkynyl, aralkyl, and heteroaralkyl groups, may also be substituted with ═O, or ═S.

When a heterocyclyl, heteroaryl, or heteroaralkyl group contains a nitrogen atom, it may be substituted or unsubstituted. When a nitrogen atom in the aromatic ring of a heteroaryl group has a substituent the nitrogen may be a quaternary nitrogen.

As used herein, the term “lower” refers to a group having up to four atoms. For example, a “lower alkyl” refers to an alkyl radical having from 1 to 4 carbon atoms, “lower alkoxy” refers to “—O—(C₁-C₄)alkyl and a “lower alkenyl” or “lower alkynyl” refers to an alkenyl or alkynyl radical having from 2 to 4 carbon atoms, respectively.

Unless indicated otherwise, the compounds of the invention containing reactive functional groups (such as (without limitation) carboxy, hydroxy, thiol, and amino moieties) also include protected derivatives thereof “Protected derivatives” are those compounds in which a reactive site or sites are blocked with one or more protecting groups. Examples of suitable protecting groups for hydroxyl groups include benzyl, methoxymethyl, allyl, trimethylsilyl, tert-butyldimethylsilyl, acetate, and the like. Examples of suitable amine protecting groups include benzyloxycarbonyl, tert-butoxycarbonyl, tert-butyl, benzyl and fluorenylmethyloxy-carbonyl (Fmoc). Examples of suitable thiol protecting groups include benzyl, tert-butyl, acetyl, methoxymethyl and the like. Other suitable protecting groups are well known to those of ordinary skill in the art and include those found in T. W. Greene, Protecting Groups in Organic Synthesis, John Wiley & Sons, Inc. 1981.

Exemplary Hsp90 inhibitors include those disclosed in U.S. Pat. Nos. 8,362,055 and 7,825,148. Examples of such compounds include AUY-922:

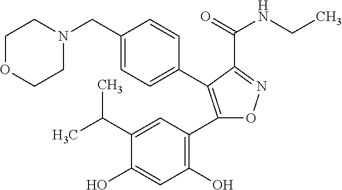 [SMILES: CCNC(=O)c1noc(-c2cc(C(C)C)c(O)cc2O)c1-c1ccc(CN2CCOCC2)cc1]

In various embodiments, the binding moiety can be an Hsp90-binding compound of formula (IV):

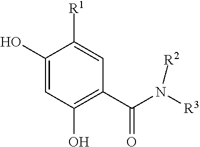

wherein

R¹ may be alkyl, aryl, halo, carboxamido or sulfonamido; R² and R³ are independently C₁-C₅ hydrocarbyl groups optionally substituted with one or more of hydroxy, halogen, C₁-C₂ alkoxy, amino, mono- and di-C₁-C₂ alkylamino; 5- to 12-membered aryl or heteroaryl groups; or, R² and R³, taken together with the nitrogen atom to which they are attached, form a 4- to 8-membered monocyclic heterocyclic group, of which up to 5 ring members are selected from O, N and S. Examples of such compounds include AT-13387:

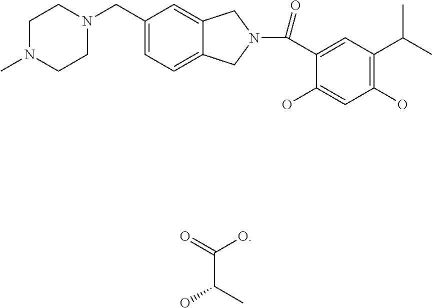

In various embodiments, the binding moiety includes an Hsp90-targeting moiety, for example one or more geldanamycins, e.g., IPI-493

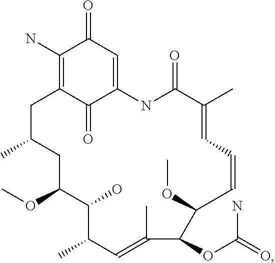

macbecins, tripterins, tanespimycins, e.g., 17-AAG

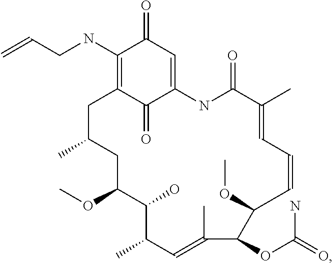 [SMILES: C=CCNC1=C2C[C@@H](C)C[C@H](OC)[C@H](O)[C@@H](C)/C=C(\C)[C@H](OC(N)=O)[C@@H](OC)/C=C\C=C(/C)C(=O)NC(=CC1=O)C2=O]

### KF-55823

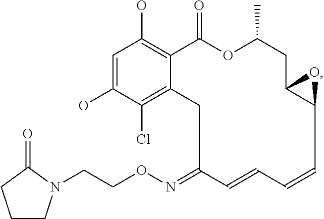 [SMILES: C[C@@H]1C[C@@H]2O[C@H]2\C=C/C=C/C(=N/OCCN2CCCC2=O)Cc2c(Cl)c(O)cc(O)c2C(=O)O1]

radicicols, KF-58333

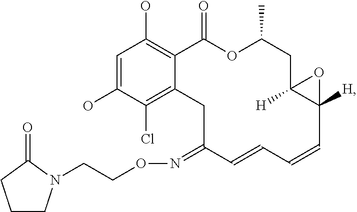

### KF-58332

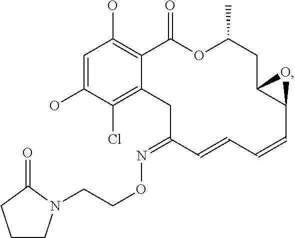 [SMILES: C[C@@H]1C[C@@H]2O[C@H]2\C=C/C=C/C(=N\OCCN2CCCC2=O)Cc2c(Cl)c(O)cc(O)c2C(=O)O1]

### 17-DMAG

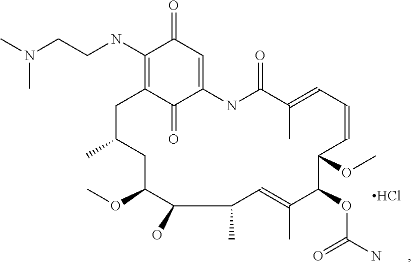 [SMILES: CO[C@H]1/C=C\C=C(/C)C(=O)NC2=CC(=O)C(NCCN(C)C)=C(C[C@@H](C)C[C@H](OC)[C@H](O)[C@@H](C)/C=C(\C)[C@@H]1OC(N)=O)C2=O ; Cl]

### IPI-504

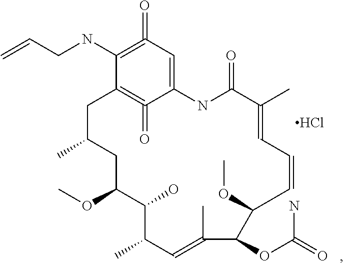

### BIIB-021

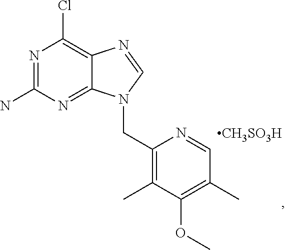

### BIIB-028, PU-H64,

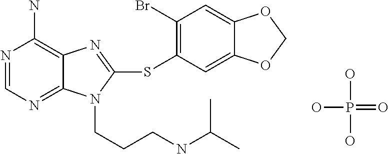 [SMILES: CC(C)NCCCn1c(Sc2cc3c(cc2Br)OCO3)nc2c(N)ncnc21 ; O=P(O)(O)O]

### PU-H71

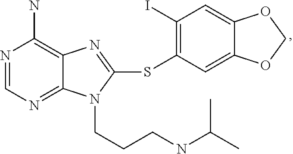 [SMILES: CC(C)NCCCn1c(Sc2cc3c(cc2I)OCO3)nc2c(N)ncnc21]

### PU-DZ8,

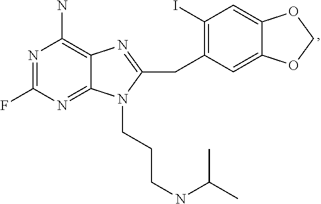 [SMILES: CC(C)NCCCn1c(Cc2cc3c(cc2I)OCO3)nc2c(N)nc(F)nc21]

### PU-HZ151

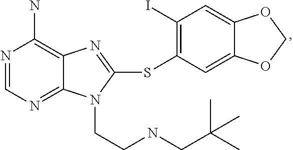 [SMILES: CC(C)(C)CNCCn1c(Sc2cc3c(cc2I)OCO3)nc2c(N)ncnc21]

### SNX-2112

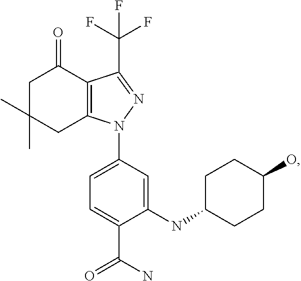

### SNX-2321

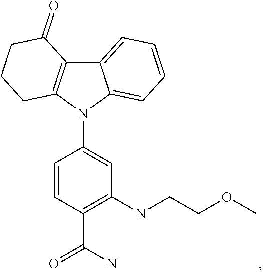 [SMILES: COCCNc1cc(-n2c3c(c4ccccc42)C(=O)CCC3)ccc1C(N)=O]

### SNX-5422

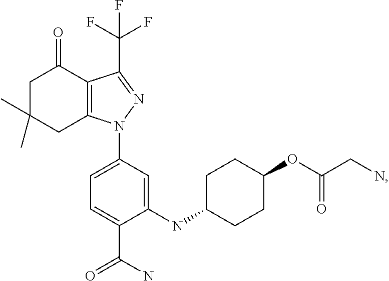

### SNX-7081

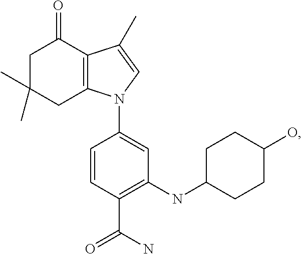

### SNX-8891, SNX-0723

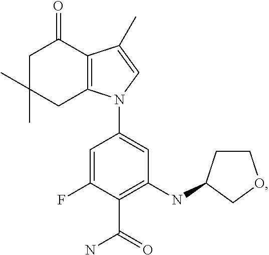 [SMILES: Cc1cn(-c2cc(F)c(C(N)=O)c(N[C@H]3CCOC3)c2)c2c1C(=O)CC(C)(C)C2]

### SAR-567530, ABI-287, ABI-328,

### AT-13387

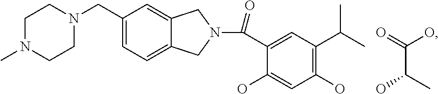

### NSC-113497

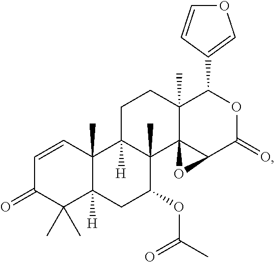

### PF-3823863

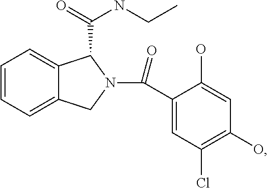

### PF-4470296

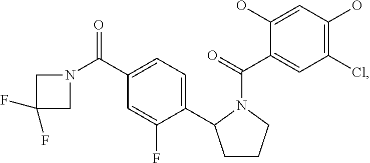 [SMILES: O=C(c1ccc(C2CCCN2C(=O)c2cc(Cl)c(O)cc2O)c(F)c1)N1CC(F)(F)C1]

### EC-102, EC-154, ARQ-250-RP, BC-274

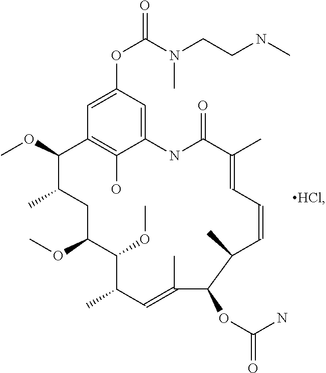

### VER-50589

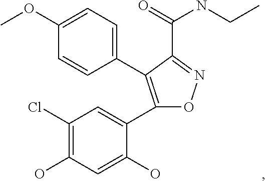 [SMILES: CCNC(=O)c1noc(-c2cc(Cl)c(O)cc2O)c1-c1ccc(OC)cc1]

### KW-2478

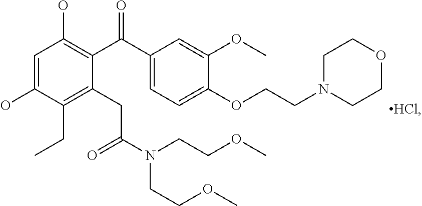 [SMILES: CCc1c(O)cc(O)c(C(=O)c2ccc(OCCN3CCOCC3)c(OC)c2)c1CC(=O)N(CCOC)CCOC]

### BHI-001,

### AUY-922

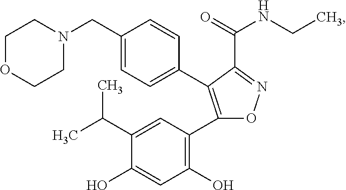 [SMILES: CCNC(=O)c1noc(-c2cc(C(C)C)c(O)cc2O)c1-c1ccc(CN2CCOCC2)cc1]

### EMD-614684

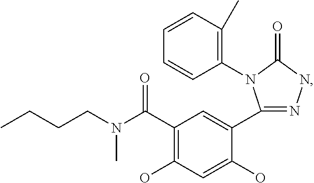

### EMD-683671, XL-888, VER-51047

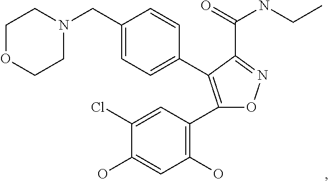 [SMILES: CCNC(=O)c1noc(-c2cc(Cl)c(O)cc2O)c1-c1ccc(CN2CCOCC2)cc1]

### KOS-2484, KOS-2539, CUDC-305

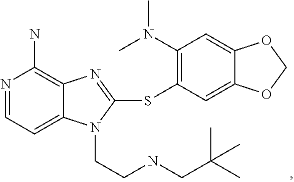 [SMILES: CN(C)c1cc2c(cc1Sc1nc3c(N)nccc3n1CCNCC(C)(C)C)OCO2]

### MPC-3100

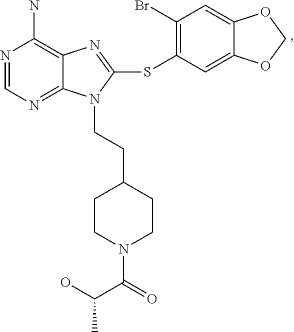 [SMILES: C[C@H](O)C(=O)N1CCC(CCn2c(Sc3cc4c(cc3Br)OCO4)nc3c(N)ncnc32)CC1]

### CH-5164840

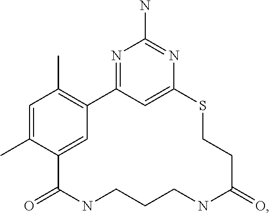 [SMILES: Cc1cc(C)c2cc1C(=O)NCCCNC(=O)CCSc1cc-2nc(N)n1]

### PU-DZ13

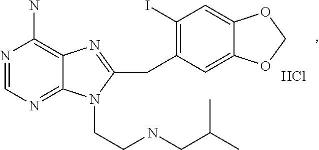 [SMILES: CC(C)CNCCn1c(Cc2cc3c(cc2I)OCO3)nc2c(N)ncnc21 ; Cl]

### PU-HZ151

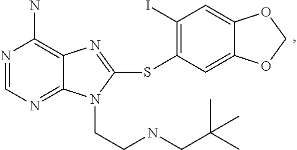 [SMILES: CC(C)(C)CNCCn1c(Sc2cc3c(cc2I)OCO3)nc2c(N)ncnc21]

### PU-DZ13

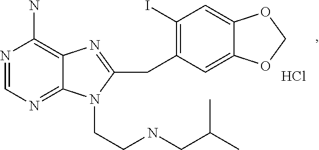 [SMILES: CC(C)CNCCn1c(Cc2cc3c(cc2I)OCO3)nc2c(N)ncnc21 ; Cl]

### VER-82576

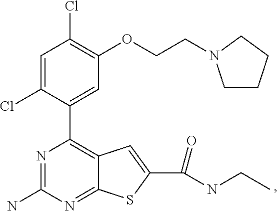 [SMILES: CCNC(=O)c1cc2c(-c3cc(OCCN4CCCC4)c(Cl)cc3Cl)nc(N)nc2s1]

### VER-82160

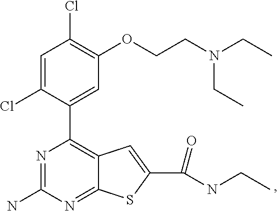 [SMILES: CCNC(=O)c1cc2c(-c3cc(OCCN(CC)CC)c(Cl)cc3Cl)nc(N)nc2s1]

### VER-82576

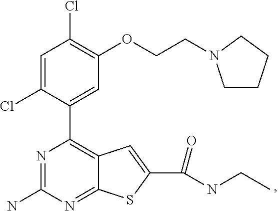 [SMILES: CCNC(=O)c1cc2c(-c3cc(OCCN4CCCC4)c(Cl)cc3Cl)nc(N)nc2s1]

### VER-82160

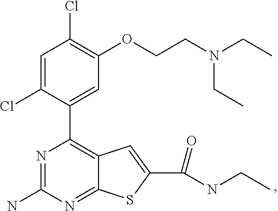 [SMILES: CCNC(=O)c1cc2c(-c3cc(OCCN(CC)CC)c(Cl)cc3Cl)nc(N)nc2s1]

### NXD-30001

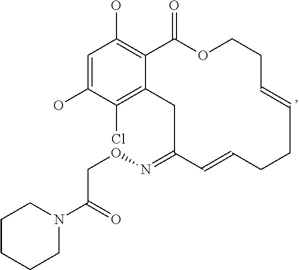

### NVP-HSP990

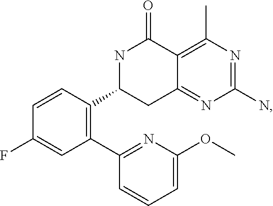

### SST-0201CL1

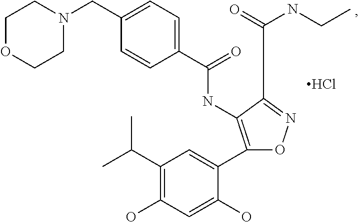 [SMILES: CCNC(=O)c1noc(-c2cc(C(C)C)c(O)cc2O)c1NC(=O)c1ccc(CN2CCOCC2)cc1 ; Cl]

### SST-0115AA1

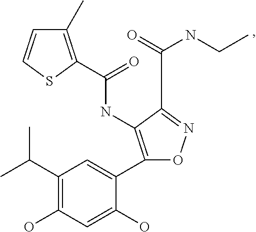 [SMILES: CCNC(=O)c1noc(-c2cc(C(C)C)c(O)cc2O)c1NC(=O)c1sccc1C]

### SST-0221AA1

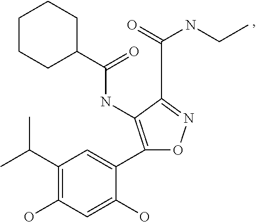 [SMILES: CCNC(=O)c1noc(-c2cc(C(C)C)c(O)cc2O)c1NC(=O)C1CCCCC1]

### SST-0223AA1

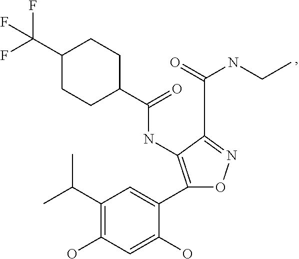 [SMILES: CCNC(=O)c1noc(-c2cc(C(C)C)c(O)cc2O)c1NC(=O)C1CCC(C(F)(F)F)CC1]

novobiocin (a C-terminal Hsp90i.)

The peptide epoxy ketone protease inhibitor in the SDC-TRAP conjugates disclosed herein are, e.g., as set forth in U.S. Pat. No. 6,831,099, and those described in U.S. Pat. Nos. 7,687,456; 7,737,112; 7,232,818; 7,417,042; 8,080,576; 8,088,741; and 8,357,683; or pharmaceutically acceptable salts, analogs, or fragments thereof.

In various embodiments, the binding moiety and the peptide epoxy ketone protease inhibitor are covalently attached. The binding moiety and the peptide epoxy ketone protease inhibitor can be covalently attached, for example by a linker. The linker can comprise a cleavable linker. The cleavable linker can comprise an enzymatically cleavable linker. The linker can be selected from the group consisting of disulfide, carbamate, amide, ester, and ether linkers.

In various embodiments, methods are featured for treating diseases or conditions including cancer, autoimmune disease, graft or transplant-related condition, neurodegenerative disease, fibrotic-associated condition, ischemic-related conditions, infection (viral, parasitic or prokaryotic) and diseases associated with bone loss, wherein a therapeutically effective amount of at least one SDC-TRAP described herein is administered to a patient. In one aspect, methods for treating cancer (e.g., multiple myeloma, e.g., multiple myeloma that is relapsed and/or refractory) in a patient are set forth, wherein a therapeutically effective amount of at least one SDC-TRAP described herein is administered to a patient.

The present invention is described in further detail by the figures and examples below, which are used only for illustration purposes and are not limiting.

### DETAILED DESCRIPTION OF THE INVENTION

The present invention provides molecules including a peptide epoxy ketone protease inhibitor conjugated to a binding moiety that directs the peptide epoxy ketone protease inhibitor to a biological target of interest. The molecules of the invention allow for selective targeting of a peptide epoxy ketone protease inhibitor by trapping the molecules of the invention in a desired cell, e.g., a cancer cell. The molecules can be described as Small molecule Drug Conjugates that are TRAPped intracellularly (SDC-TRAP), due to their selective binding to high concentration intracellular proteins. In order for the molecules of the invention to be trapped within the cells of interest, the binding moieties that are part of the SDC-TRAP molecules interact with proteins that are overexpressed in targeted cells. In exemplary embodiments, the proteins that are overexpressed are characteristic of a particular disease or disorder. Accordingly, the present invention provides compositions, kits, and methods (e.g., therapeutic, diagnostic, and imaging) that include the molecules of the invention. Using the targeted delivery molecules described herein (SDC-TRAPs) allows, in select cases, for peptide epoxy ketone protease inhibitors to be dosed at lower levels, thereby allowing the toxic effector to be targeted to specific diseased cells at sub-toxic levels.

In various aspects and embodiments, SDC-TRAPs comprising a target protein-interacting binding moiety. A target protein-interacting binding moiety can selectively interact with any one or more domains of a target protein. For example, where a target protein is Hsp90, the binding moiety can be an Hsp90 binding moiety that interacts with the N-terminal domain of Hsp90, the C-terminal domain of Hsp90, and/or the middle domain of Hsp90. Selective interaction with any one or more domains of a target protein can advantageously increase specificity and/or increase the concentration of molecular targets within a target tissue and/or cell.

In various aspects and embodiments, the present invention provides an SDC-TRAP comprising a binding moiety having a high affinity for a molecular target (e.g., a K_{d} of 50, 100, 150, 200, 250, 300, 350, 400 nM or higher). For example, where a binding moiety is an Hsp90 binding moiety, the Hsp90 binding moiety can have a K_{d} of 50, 100, 150, 200, 250, 300, 350, 400 nM or higher. A binding moiety having a high affinity for a molecular target can advantageously improve targeting and/or increase the resonance time of the SDC-TRAP in a target cell and/or tissue.

In various aspects and embodiments, the present invention provides an SDC-TRAP comprising a binding moiety (e.g., Hsp90 binding moiety) and a peptide epoxy ketone protease inhibitor, wherein when administered to a subject the SDC-TRAP is present at a ratio of about 2:1 in tumor cells compared to plasma. The ratio can be higher, for example, about 5:1, 10:1, 25:1, 50:1, 75:1, 100:1, 150:1, 200:1, 250:1, 300:1, 400:1, 500:1, 600:1, 700:1, 800:1, 900:1, 1000:1, or greater. In various aspects and embodiments, the ratio is at 1, 2, 3, 4, 5, 6, 7, 8, 12, 24, 48, 72, or more hours from administration. The effectiveness of targeting can be reflected in the ratio of SDC-TRAP in a target cell and/or tissue compared to plasma.

In various aspects and embodiments, the present invention provides an SDC-TRAP comprising a binding moiety (e.g., Hsp90 binding moiety) and a peptide epoxy ketone protease inhibitor, wherein the SDC-TRAP is present in the target (e.g., cancer) cells for at least 24 hours. The SDC-TRAP can be present in cancer cells for longer, for example, for at least 48, 72, 96, or 120 hours. It can be advantageous for an SDC-TRAP to be present in target cells for longer periods of time to increase the therapeutic effect of a given dose of SDC-TRAP and/or increase an interval between administrations of SDC-TRAP.

In various aspects and embodiments, the present invention provides an SDC-TRAP comprising a binding moiety (e.g., Hsp90 binding moiety) and a peptide epoxy ketone protease inhibitor, wherein the peptide epoxy ketone protease inhibitor is released for a period of at least 6 hours. The peptide epoxy ketone protease inhibitor can be released for a longer period, for example, for at least 12, 24, 48, 72, 96, or 120 hours. Selective release can be used to control, delay, and/or extend the period of release of a peptide epoxy ketone protease inhibitor and, therefore, increase the therapeutic effect of a given dose of SDC-TRAP, decrease the undesired side effects of a given dose of SDC-TRAP, and/or increase an interval between administrations of SDC-TRAP.

In various aspects and embodiments, the present invention provides an SDC-TRAP comprising an Hsp90 binding moiety and a peptide epoxy ketone protease inhibitor, wherein the peptide epoxy ketone protease inhibitor is selectively released inside a target (e.g., cancer) cell. Selective release can be achieved, for example, by a cleavable linker (e.g., an enzymatically cleavable linker). Selective release can be used to decrease undesired toxicity and/or unwanted side effects.

In various aspects and embodiments, the SDC-TRAP can exhibit decreased and/or minimized toxicity concurrently with increased efficacy (e.g., as compared to that of the peptide epoxy ketone protease inhibitor when used alone). Decreasing and/or minimizing toxicity can encompass reducing toxicity to a predetermined level (e.g., a regulatory guideline or suggested level.) Increasing efficacy can encompass increasing efficacy to a predetermined level. Similarly, decreasing and/or minimizing toxicity concurrently with increasing efficacy can encompass achieving a predetermined therapeutic ratio.

Decreasing and/or minimizing toxicity can encompass, for example, reducing toxicity by 5, 10, 15, 20, 25, 30, 35, 40, 45, 50, 55, 60, 65, 70, 75, 80, 85, 90, 95%, or more. Increasing efficacy can encompass, for example, increasing efficacy by 5, 10, 15, 20, 25, 30, 35, 40, 45, 50, 55, 60, 65, 70, 75, 80, 85, 90, 95, 100, 125, 150, 175, 200, 250, 300, 400, 500%, or more. Decreasing and/or minimizing toxicity concurrently with increasing efficacy can encompass, for example: essentially the same efficacy with decreased toxicity; essentially the same toxicity with increased efficacy; or decreased toxicity and increased efficacy. Similarly, decreasing and/or minimizing toxicity concurrently with increasing efficacy can encompass, for example, scenarios such as: increased efficacy enabling a lower dose (e.g., lower dose of peptide epoxy ketone protease inhibitor with a correspondingly lower net toxicity) and decreased toxicity enabling a higher dose (e.g., higher dose of peptide epoxy ketone protease inhibitor without a correspondingly higher net toxicity).

In various aspects and embodiments, the present invention provides an SDC-TRAP comprising a binding moiety (e.g., Hsp90 binding moiety) and a peptide epoxy ketone protease inhibitor, wherein the binding moiety is an inhibitor (e.g., Hsp90 inhibitor) that is ineffective as a therapeutic agent when administered alone. In such cases, the SDC-TRAP may facilitate an additive or synergistic effect between the binding moiety and peptide epoxy ketone protease inhibitor, thereby advantageously improving the efficacy and/or reducing the side effects of a therapy.

In order that the present invention may be more readily understood, certain terms are first defined. In addition, it should be noted that whenever a value or range of values of a parameter are recited, it is intended that values and ranges intermediate to the recited values are also intended to be part of this invention. Unless defined otherwise, all technical and scientific terms used herein have the same meaning as commonly understood to one of ordinary skill in the art to which this invention belongs. It is also to be understood that the terminology employed is for the purpose of describing particular embodiments, and is not intended to be limiting.

### DEFINITIONS

The articles “a,” “an,” and “the” are used herein to refer to one or to more than one (i.e. to at least one) of the grammatical object of the article unless otherwise clearly indicated by contrast. By way of example, “an element” means one element or more than one element.

The term “including” is used herein to mean, and is used interchangeably with, the phrase “including but not limited to.”

The term “or” is used herein to mean, and is used interchangeably with, the term “and/or,” unless context clearly indicates otherwise.

The term “such as” is used herein to mean, and is used interchangeably, with the phrase “such as but not limited to.”

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of a listing of chemical group(s) in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

As used herein, the term “subject” refers to human and non-human animals, including veterinary subjects. The term “non-human animal” includes all vertebrates, e.g., mammals and non-mammals, such as non-human primates, mice, rabbits, sheep, dog, cat, horse, cow, chickens, amphibians, and reptiles. In a preferred embodiment, the subject is a human and may be referred to as a patient.

As used herein, the terms “treat,” “treating” or “treatment” refer, preferably, to an action to obtain a beneficial or desired clinical result including, but not limited to, alleviation or amelioration of one or more signs or symptoms of a disease or condition, diminishing the extent of disease, stability (i.e., not worsening) state of disease, amelioration or palliation of the disease state, diminishing rate of or time to progression, and remission (whether partial or total), whether detectable or undetectable. “Treatment” can also mean prolonging survival as compared to expected survival in the absence of treatment. Treatment does not need to be curative.

A “therapeutically effective amount” is that amount sufficient to treat a disease in a subject. A therapeutically effective amount can be administered in one or more administrations.

By “diagnosing” and the like, as used herein, refers to a clinical or other assessment of the condition of a subject based on observation, testing, or circumstances for identifying a subject having a disease, disorder, or condition based on the presence of at least one indicator, such as a sign or symptom of the disease, disorder, or condition. Typically, diagnosing using the method of the invention includes the observation of the subject for multiple indicators of the disease, disorder, or condition in conjunction with the methods provided herein. Diagnostic methods provide an indicator that a disease is or is not present. A single diagnostic test typically does not provide a definitive conclusion regarding the disease state of the subject being tested.

The terms “administer,” “administering” or “administration” include any method of delivery of a pharmaceutical composition or agent into a subject's system or to a particular region in or on a subject. In certain embodiments of the invention, an agent is administered intravenously, intramuscularly, subcutaneously, intradermally, intranasally, orally, transcutaneously, or mucosally. In a preferred embodiment, an agent is administered intravenously. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, intratumoral delivery, etc.

As used herein, the term “survival” refers to the continuation of life of a subject which has been treated for a disease or condition, e.g., cancer. The time of survival can be defined from an arbitrary point such as time of entry into a clinical trial, time from completion or failure or an earlier treatment regimen, time from diagnosis, etc.

As used herein, the term “recur” refers to the re-growth of tumor or cancerous cells in a subject in whom primary treatment for the tumor has been administered. The tumor may recur in the original site or in another part of the body. In one embodiment, a tumor that recurs is of the same type as the original tumor for which the subject was treated. For example, if a subject had an ovarian cancer tumor, was treated and subsequently developed another ovarian cancer tumor, the tumor has recurred. In addition, a cancer can recur in or metastasize to a different organ or tissue than the one where it originally occurred.

As used herein, the terms “identify” or “select” refer to a choice in preference to another. In other words, to identify a subject or select a subject is to perform the active step of picking out that particular subject from a group and confirming the identity of the subject by name or other distinguishing feature.

As used herein, the term “benefit” refers to something that is advantageous or good, or an advantage. Similarly, the term “benefiting,” as used herein, refers to something that improves or advantages. For example, a subject will benefit from treatment if they exhibit a decrease in at least one sign or symptom of a disease or condition (e.g., tumor shrinkage, decrease in tumor burden, inhibition or decrease of metastasis, improving quality of life (“QOL”), if there is a delay of time to progression (“TTP”), if there is an increase of overall survival (“OS”), etc.), or if there is a slowing or stopping of disease progression (e.g., halting tumor growth or metastasis, or slowing the rate of tumor growth or metastasis). A benefit can also include an improvement in quality of life, or an increase in survival time or progression free survival.

The terms “cancer” or “tumor” are well known in the art and refer to the presence, e.g., in a subject, of cells possessing characteristics typical of cancer-causing cells, such as uncontrolled proliferation, immortality, metastatic potential, rapid growth and proliferation rate, decreased cell death/apoptosis, and certain characteristic morphological features. Cancer cells are often in the form of a solid tumor. However, cancer also includes non-solid tumors, e.g., blood tumors, e.g., leukemia, wherein the cancer cells are derived from bone marrow. As used herein, the term “cancer” includes pre-malignant as well as malignant cancers. Cancers include, but are not limited to, acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, Burkitt's lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin, and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, and Wilms' tumor. Other cancers include primary cancer, metastatic cancer, oropharyngeal cancer, hypopharyngeal cancer, liver cancer, gall bladder cancer, bile duct cancer, small intestine cancer, urinary tract cancer, kidney cancer, urothelium cancer, female genital tract cancer, uterine cancer, gestational trophoblastic disease, male genital tract cancer, seminal vesicle cancer, testicular cancer, germ cell tumors, endocrine gland tumors, thyroid cancer, adrenal cancer, pituitary gland cancer, hemangioma, sarcoma arising from bone and soft tissues, Kaposi's sarcoma, nerve cancer, ocular cancer, meningial cancer, glioblastomas, neuromas, neuroblastomas, Schwannomas, solid tumors arising from hematopoietic malignancies such as leukemias, metastatic melanoma, recurrent or persistent ovarian epithelial cancer, fallopian tube cancer, primary peritoneal cancer, gastrointestinal stromal tumors, colorectal cancer, gastric cancer, melanoma, glioblastoma multiforme, non-squamous non-small-cell lung cancer, malignant glioma, epithelial ovarian cancer, primary peritoneal serous cancer, metastatic liver cancer, neuroendocrine carcinoma, refractory malignancy, triple negative breast cancer, HER2-amplified breast cancer, nasopharageal cancer, oral cancer, biliary tract, hepatocellular carcinoma, squamous cell carcinomas of the head and neck (SCCHN), non-medullary thyroid carcinoma, recurrent glioblastoma multiforme, neurofibromatosis type 1, CNS cancer, liposarcoma, leiomyosarcoma, salivary gland cancer, mucosal melanoma, acral/lentiginous melanoma, paraganglioma, pheochromocytoma, advanced metastatic cancer, solid tumor, triple negative breast cancer, colorectal cancer, sarcoma, melanoma, renal carcinoma, endometrial cancer, thyroid cancer, rhabdomysarcoma, multiple myeloma, ovarian cancer, glioblastoma, gastrointestinal stromal tumor, mantle cell lymphoma, and refractory malignancy.

“Solid tumor,” as used herein, is understood as any pathogenic tumor that can be palpated or detected using imaging methods as an abnormal growth having three dimensions. A solid tumor is differentiated from a blood tumor such as leukemia. However, cells of a blood tumor are derived from bone marrow; therefore, the tissue producing the cancer cells is a solid tissue that can be hypoxic.

“Tumor tissue” is understood as cells, extracellular matrix, and other naturally occurring components associated with the solid tumor.

As used herein, the term “isolated” refers to a preparation that is substantially free (e.g., 50%, 60%, 70%, 80%, 90% or more, by weight) from other proteins, nucleic acids, or compounds associated with the tissue from which the preparation is obtained.

The term “sample” as used herein refers to a collection of similar fluids, cells, or tissues isolated from a subject. The term “sample” includes any body fluid (e.g., urine, serum, blood fluids, lymph, gynecological fluids, cystic fluid, ascetic fluid, ocular fluids, and fluids collected by bronchial lavage and/or peritoneal rinsing), ascites, tissue samples (e.g., tumor samples) or a cell from a subject. Other subject samples include tear drops, serum, cerebrospinal fluid, feces, sputum, and cell extracts. In one embodiment, the sample is removed from the subject. In a particular embodiment, the sample is urine or serum. In another embodiment, the sample does not include ascites or is not an ascites sample. In another embodiment, the sample does not include peritoneal fluid or is not peritoneal fluid. In one embodiment, the sample comprises cells. In another embodiment, the sample does not comprise cells. Samples are typically removed from the subject prior to analysis. However, tumor samples can be analyzed in the subject, for example, using imaging or other detection methods.

The term “control sample,” as used herein, refers to any clinically relevant comparative sample, including, for example, a sample from a healthy subject not afflicted with cancer, a sample from a subject having a less severe or slower progressing cancer than the subject to be assessed, a sample from a subject having some other type of cancer or disease, a sample from a subject prior to treatment, a sample of non-diseased tissue (e.g., non-tumor tissue), a sample from the same origin and close to the tumor site, and the like. A control sample can be a purified sample, protein, and/or nucleic acid provided with a kit. Such control samples can be diluted, for example, in a dilution series to allow for quantitative measurement of analytes in test samples. A control sample may include a sample derived from one or more subjects. A control sample may also be a sample made at an earlier time point from the subject to be assessed. For example, the control sample could be a sample taken from the subject to be assessed before the onset of the cancer, at an earlier stage of disease, or before the administration of treatment or of a portion of treatment. The control sample may also be a sample from an animal model, or from a tissue or cell lines derived from the animal model, of the cancer. The level in a control sample that consists of a group of measurements may be determined, e.g., based on any appropriate statistical measure, such as, for example, measures of central tendency including average, median, or modal values.

As used herein, the term “obtaining” is understood herein as manufacturing, purchasing, or otherwise coming into possession of.

As used herein, the term “identical” or “identity” is used herein in relation to amino acid or nucleic acid sequences refers to any gene or protein sequence that bears at least 30% identity, more preferably 40%, 50%, 60%, 70%, 75%, 80%, 81%, 82%, 83%, 84%, 85%, 86%, 87%, 88%, 89%, 90%, 91%, 92%, 93%, 94%, and most preferably 95%, 96%, 97%, 98%, 99% or more identity to a known gene or protein sequence over the length of the comparison sequence. Protein or nucleic acid sequences with high levels of identity throughout the sequence can be said to be homologous. A “homologous” protein can also have at least one biological activity of the comparison protein. In general, for proteins, the length of comparison sequences will be at least 10 amino acids, preferably 10, 20, 30, 40, 50, 60, 70, 80, 90, 100, 150, 175, 200, 250, or at least 300 amino acids or more. For nucleic acids, the length of comparison sequences will generally be at least 25, 50, 100, 125, 150, 200, 250, 300, 350, 400, 450, 500, 550, 600, 650, 700, 800, or at least 850 nucleotides or more.

As used herein, “detecting,” “detection” and the like are understood that an assay performed for identification of a specific analyte in a sample. The amount of analyte or activity detected in the sample can be none or below the level of detection of the assay or method.

The terms “modulate” or “modulation” refer to upregulation (i.e., activation or stimulation), downregulation (i.e., inhibition or suppression) of a level, or the two in combination. A “modulator” is a compound or molecule that modulates, and may be, e.g., an agonist, antagonist, activator, stimulator, suppressor, or inhibitor.

The term “expression” is used herein to mean the process by which a polypeptide is produced from DNA. The process involves the transcription of the gene into mRNA and the translation of this mRNA into a polypeptide. Depending on the context in which used, “expression” may refer to the production of RNA, or protein, or both.

The terms “level of expression of a gene” or “gene expression level” refer to the level of mRNA, as well as pre-mRNA nascent transcript(s), transcript processing intermediates, mature mRNA(s) and degradation products, or the level of protein, encoded by the gene in the cell.

As used herein, “level of activity” is understood as the amount of protein activity, typically enzymatic activity, as determined by a quantitative, semi-quantitative, or qualitative assay. Activity is typically determined by monitoring the amount of product produced in an assay using a substrate that produces a readily detectable product, e.g., colored product, fluorescent product, or radioactive product.

As used herein, “changed as compared to a control” sample or subject is understood as having a level of the analyte or diagnostic or therapeutic indicator (e.g., marker) to be detected at a level that is statistically different than a sample from a normal, untreated, or control sample control samples include, for example, cells in culture, one or more laboratory test animals, or one or more human subjects. Methods to select and test control samples are within the ability of those skilled in the art. An analyte can be a naturally occurring substance that is characteristically expressed or produced by the cell or organism (e.g., an antibody, a protein) or a substance produced by a reporter construct (e.g., β-galactosidase or luciferase). Depending on the method used for detection the amount and measurement of the change can vary. Changed as compared to a control reference sample can also include a change in one or more signs or symptoms associated with or diagnostic of disease, e.g., cancer. Determination of statistical significance is within the ability of those skilled in the art, e.g., the number of standard deviations from the mean that constitute a positive result.

“Elevated” or “lower” refers to a patient's value of a marker relative to the upper limit of normal (“ULN”) or the lower limit of normal (“LLN”) which are based on historical normal control samples. As the level of the marker present in the subject will be a result of the disease, and not a result of treatment, typically a control sample obtained from the patient prior to onset of the disease will not likely be available. Because different labs may have different absolute results, values are presented relative to that lab's upper limit of normal value (ULN).

The “normal” level of expression of a marker is the level of expression of the marker in cells of a subject or patient not afflicted with cancer. In one embodiment, a “normal” level of expression refers to the level of expression of the marker under normoxic conditions.

An “over-expression” or “high level of expression” of a marker refers to an expression level in a test sample that is greater than the standard error of the assay employed to assess expression, and is preferably at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3, 4, 5, 6, 7, 8, 9, or 10 times the expression level of the marker in a control sample (e.g., sample from a healthy subject not having the marker associated disease, i.e., cancer). In one embodiment, expression of a marker is compared to an average expression level of the marker in several control samples.

A “low level of expression” or “under-expression” of a marker refers to an expression level in a test sample that is less than at least 0.9, 0.8, 0.7, 0.6, 0.5, 0.4, 0.3, 0.2, or 0. 1 times the expression level of the marker in a control sample (e.g., sample from a healthy subject not having the marker associated disease, i.e., cancer). In one embodiment, expression of a marker is compared to an average expression level of the marker in several control samples.

As used herein, “binding” is understood as having at least a 10² or more, 10³ or more, preferably 10⁴ or more, preferably 10⁵ or more, preferably 10⁶ or more preference for binding to a specific binding partner as compared to a non-specific binding partner (e.g., binding an antigen to a sample known to contain the cognate antibody).

“Determining” as used herein is understood as performing an assay or using a diagnostic method to ascertain the state of someone or something, e.g., the presence, absence, level, or degree of a certain condition, biomarker, disease state, or physiological condition.

“Prescribing” as used herein is understood as indicating a specific agent or agents for administration to a subject.

As used herein, the terms “respond” or “response” are understood as having a positive response to treatment with a therapeutic agent, wherein a positive response is understood as having a decrease in at least one sign or symptom of a disease or condition (e.g., tumor shrinkage, decrease in tumor burden, inhibition or decrease of metastasis, improving quality of life (“QOL”), delay of time to progression (“TTP”), increase of overall survival (“OS”), etc.), or slowing or stopping of disease progression (e.g., halting tumor growth or metastasis, or slowing the rate of tumor growth or metastasis). A response can also include an improvement in quality of life, or an increase in survival time or progression free survival.

The terms “administer,” “administering” or “administration” can include any method of delivery of a pharmaceutical composition or agent into a subject's system or to a particular region in or on a subject. In certain embodiments of the invention, an Hsp90 inhibitor is administered intravenously, intramuscularly, subcutaneously, intradermally, intranasally, orally, transcutaneously, or mucosally. In a preferred embodiment, an agent is administered intravenously. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, intratumoral delivery, etc.

As used herein, the term “high concentration” refers to the concentration of SDC-TRAP that accumulates in target cells of the invention due to the selective binding of the binding moiety of the SDC-TRAP to the target protein. In one embodiment, the concentration is higher than in similar cells that do not overexpress the target protein, e.g., lung cancer cells as compared to non-cancerous lung cells. In another embodiment, the concentration is higher in target cells compared to cells that do not express, or overexpress, the target protein. In exemplary embodiments, the high concentration is 1.5, 2, 3, 4, 5, 10, 15, 20, 50, 100, 1000 times or more than cells that are not targeted by the SDC-TRAP molecules of the invention.

The term “moiety” refers generally to a portion of a molecule, which may be a functional group, a set of functional groups, and/or a specific group of atoms within a molecule, that is responsible for a characteristic chemical, biological, and/or medicinal property of the molecule.

The term “binding moiety” refers to low molecular weight (e.g., less than about 800, 700, 600, 500, 400, 300, 200, or 100 etc. Dalton) organic compounds, which may serve as a therapeutic or a regulator of a biological process. Binding moieties include molecules that can bind to a biopolymer such as protein, nucleic acid, or polysaccharide and acts as an effector, altering the activity or function of the biopolymer. Binding moieties can have a variety of biological functions, serving as cell signaling molecules, as tools in molecular biology, as drugs in medicine, as pesticides in farming, and in many other roles. These compounds can be natural (such as secondary metabolites) or artificial (such as antiviral drugs); they may have a beneficial effect against a disease (such as drugs) or may be detrimental (such as teratogens and carcinogens). Biopolymers such as nucleic acids, proteins, and polysaccharides (such as starch or cellulose) are not binding moieties, although their constituent monomers—ribo- or deoxyribo-nucleotides, amino acids, and monosaccharides, respectively—are often considered to be. Small oligomers are also usually considered binding moieties, such as dinucleotides, peptides such as the antioxidant glutathione, and disaccharides such as sucrose.

As used herein, a “protein interacting binding moiety” or “binding moiety” refers to a binding moiety, or portion thereof, that interacts with a predetermined target. The interaction is achieved through some degree of specificity and/or affinity for the target. Both specificity and affinity is generally desirable, although in certain cases higher specificity may compensate for lower affinity and higher affinity may compensate for lower specificity. Affinity and specificity requirements will vary depending upon various factors including, but not limited to, absolute concentration of the target, relative concentration of the target (e.g., in cancer vs. normal cells), potency and toxicity, route of administration, and/or diffusion or transport into a target cell. The target can be a molecule of interest and/or localized in an area of interest. For example, the target can be a therapeutic target and/or localized in an area targeted for a therapy (e.g., a protein that is overexpressed in cancerous cells, as compared to normal cells). In one particular example, a target can be a chaperonin protein such as Hsp90 and the binding moiety can be an Hsp90 binding moiety (e.g., therapeutic, cytotoxic, or imaging moiety). Preferentially, the binding moiety will enhance, be compatible with, or not substantially reduce, passive transport of a conjugate including the binding moiety into a cell, e.g., a cell comprising a target protein.

The term “peptide epoxy ketone protease inhibitor” refers to compounds that are analogs or prodrugs of the compounds disclosed in U.S. patent application Ser. No. 09/569,748. Suitable enzyme inhibitor analogs or prodrugs may have a structure of formula (I) or a pharmaceutically acceptable salt thereof,

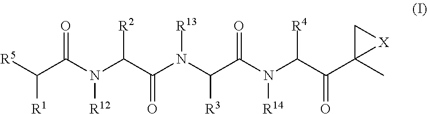

wherein each A is independently selected from C═O, C═S, and SO₂, preferably C═O; or

A is optionally a covalent bond when adjacent to an occurrence of Z;

L is absent or is selected from C═O, C═S, and SO₂, preferably L is absent or C═O;

M is absent or is C₁₋₁₂alkyl, preferably C₁₋₈alkyl;

Q is absent or is selected from O, NH, and N—C₁₋₆alkyl, preferably Q is absent, O, or NH, most preferably Q is absent or O;

X is selected from O, NH, and N—C₁₋₆alkyl, preferably O;

Y is absent or is selected from O, NH, N—C₁₋₆alkyl, S, SO, SO₂, CHOR¹⁰, and CHCO₂R¹⁰;

each Z is independently selected from O, S, NH, and N—C₁₋₆alkyl, preferably O; or

Z is optionally a covalent bond when adjacent to an occurrence of A;

R¹, R², R³, and R⁴ are each independently selected from C₁₋₆alkyl, C₁₋₆hydroxyalkyl, C₁₋₆alkoxyalkyl, aryl, and C₁₋₆aralkyl, any of which is optionally substituted with one or more of amide, amine, carboxylic acid (or a salt thereof), ester (including C₁₋₅ alkyl ester and aryl ester), thiol, or thioether substituents;

R⁵ is N(R⁶)LQR⁷;

R⁶, R¹², R¹³, and R¹⁴ are independently selected from hydrogen, OH, C₁₋₆alkyl, and a group of formula IV; preferably, R⁶ is selected from hydrogen, OH, and C₁₋₆alkyl, and R¹², R¹³, and R¹⁴ are independently selected from hydrogen and C₁₋₆alkyl, preferably hydrogen;

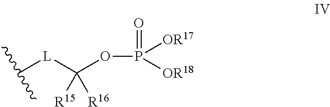

R⁷ is selected from hydrogen, C₁₋₆alkyl, C₁₋₆alkenyl, C₁₋₆alkynyl, aryl, C₁₋₆aralkyl, heteroaryl, C₁₋₆heteroaralkyl, R⁸ZAZ-C₁₋₈alkyl-, R¹¹Z—C₁₋₈alkyl-, (R⁸O)(R⁹O)P(═O)O—C₁₋₈alkyl-ZAZ-C₁₋₈ alkyl-, R⁸ZAZ—C₁₋₈ alkyl-ZAZ-C₁₋₈alkyl-, heterocyclylMZAZ-C₁₋₈alkyl-, (R⁸O)(R⁹O)P(═O)O—C₁₋₈alkyl-, (R¹⁰)₂N—C₁₋₁₂alkyl-, (R¹⁰)₃N⁺—C₁₋₁₂alkyl-, heterocyclylM-, carbocyclylM-, R¹¹SO₂C₁₋₈alkyl-, and R¹¹SO₂NH; preferably C₁₋₆alkyl, C₁₋₆alkenyl, C₁₋₆alkynyl, aryl, C₁₋₆aralkyl, heteroaryl, C₁₋₆heteroaralkyl, R⁸ZA-C₁₋₈alkyl-, R¹¹Z—C₁₋₈alkyl-, (R⁸O)(R⁹O)P(═O)O—C₁₋₈alkyl-ZAZ-C₁₋₈ alkyl-, (R⁸O)(R⁹O)P(═O)O—C₁₋₈alkyl-Z—C₁₋₈ alkyl-, R⁸ZA-C₁₋₈alkyl-ZAZ-C₁₋₈alkyl-, heterocyclyhMZAZ-C₁₋₈alkyl-, (R⁸O)(R⁹O)P(═O)O—C₁₋₈alkyl-, (R¹⁰)₂N—C₁₋₈alkyl-, (R¹⁰)₃N⁺—C₁₋₈alkyl-, heterocyclylM-, carbocyclylM-, R¹¹SO₂C₁₋₈alkyl-, and R¹¹SO₂NH, wherein each occurrence of Z and A is independently other than a covalent bond; or

R⁶ and R⁷ together are C₁₋₆alkyl-Y—C₁₋₆alkyl, C₁₋₆alkyl-ZAZ-C₁₋₆alkyl, ZAZ-C₁₋₆alkyl-ZAZ-C₁₋₆alkyl, ZAZ-C₁₋₆alkyl-ZAZ, or C₁₋₆alkyl-A, thereby forming a ring; preferably C₁₋₂alkyl-Y—C₁₋₂alkyl, C₁₋₂alkyl-ZA-C₁₋₂alkyl, A-C₁₋₂alkyl-ZA-C₁₋₂alkyl, A-C₁₋₃alkyl-A, or C₁₋₄alkyl-A, wherein each occurrence of Z and A is independently other than a covalent bond;

R⁸ and R⁹ are independently selected from hydrogen, metal cation, C₁₋₆alkyl, C₁₋₆alkenyl, C₁₋₆alkynyl, aryl, heteroaryl, C₁₋₆aralkyl, and C₁₋₆heteroaralkyl, preferably from hydrogen, metal cation, and C₁₋₆alkyl, or R⁸ and R⁹ together are C₁₋₆alkyl, thereby forming a ring;

each R¹⁰ is independently selected from hydrogen and C₁₋₆alkyl, preferably C₁₋₆alkyl; and

R¹¹ is independently selected from hydrogen, C₁₋₆alkyl, C₁₋₆alkenyl, C₁₋₆alkynyl, carbocyclyl, heterocyclyl, aryl, heteroaryl, C₁₋₆aralkyl, and C₁₋₆heteroaralkyl,

R¹⁵ and R¹⁶ are independently selected from hydrogen and C₁₋₆alkyl, or R¹⁵ and R¹⁶ together form a 3- to 6-membered carbocyclic or heterocyclic ring; and

R¹⁷ and R¹⁸ are independently selected from hydrogen, a metal cation, C₁₋₆alkyl, and C₁₋₆aralkyl, or R¹⁷ and R¹⁸ together represent C₁₋₆alkyl, thereby forming a ring;

provided that when R⁶, R¹², R¹³, and R¹⁴ are H or CH₃, and Q is absent, LR⁷ is not hydrogen, unsubstituted C₁₋₆alkylC═O, a further chain of amino acids, t-butoxycarbonyl (Boc), benzoyl (Bz), fluoren-9-ylmethoxycarbonyl (Fmoc), triphenylmethyl(trityl), benzyloxycarbonyl (Cbz), trichloroethoxycarbonyl (Troc); or substituted or unsubstituted aryl or heteroaryl; and

in any occurrence of the sequence ZAZ, at least one member of the sequence must be other than a covalent bond.

In certain embodiments, when R⁶ is H, L is C═O, and Q is absent, R⁷ is not hydrogen, C₁₋₆alkyl, or substituted or unsubstituted aryl or heteroaryl. In certain embodiments, when R⁶ is H and Q is absent, R⁷ is not a protecting group such as those described in Greene, T. W. and Wuts, P. G. M., “Protective Groups in Organic Synthesis”, John Wiley & Sons, 1999 or Kocienfski, P. J., “Protecting Groups”, Georg Thieme Verlag, 1994.

In some embodiments, R¹, R², R³, and R⁴ are selected from C₁₋₆alkyl or C₁₋₆aralkyl. In preferred embodiments, R² and R⁴ are C₁₋₆alkyl and R¹ and R³ are C₁₋₆aralkyl. In the most preferred embodiment, R² and R⁴ are isobutyl, R¹ is 2-phenylethyl, and R³ is phenylmethyl.

In certain embodiments, L and Q are absent and R⁷ is selected from C₁₋₆alkyl, C₁₋₆alkenyl, C₁₋₆alkynyl, C₁₋₆aralkyl, and C₁₋₆heteroaralkyl. In certain such embodiments, R⁶ is C₁₋₆alkyl and R⁷ is selected from butyl, allyl, propargyl, phenylmethyl, 2-pyridyl, 3-pyridyl, and 4-pyridyl.

In other embodiments, L is SO₂, Q is absent, and R⁷ is selected from C₁₋₆alkyl and aryl. In certain such embodiments, R⁷ is selected from methyl and phenyl.

In certain embodiments, L is C═O and R⁷ is selected from C₁₋₆alkyl, C₁₋₆alkenyl, C₁₋₆alkynyl, aryl, C₁₋₆aralkyl, heteroaryl, C₁₋₆heteroaralkyl, R⁸ZA-C₁₋₈alkyl-R¹¹Z—C₁₋₈alkyl-, (R⁸O)(R⁹O)P(═O)O—C₁₋₈alkyl-, (R⁸O)(R⁹O)P(═O)O—C₁₋₈alkyl-ZAZ-C₁₋₈ alkyl-, (R⁸O)(R⁹O)P(═O)O—C₁₋₈alkyl-Z—C₁₋₈ alkyl-, R⁸ZA-C₁₋₈alkyl-ZAZ-C₁₋₈alkyl-, heterocyclylMZAZ-C₁₋₈alkyl-, (R¹⁰)₂N—C₁₋₈alkyl-, (R¹⁰)₃N⁺—C₁₋₈alkyl-, heterocyclyl-M carbocyclylM-, R¹¹SO₂C₁₋₈alkyl-, and R¹¹SO₂NH—, wherein each occurrence of Z and A is independently other than a covalent bond. In certain embodiments, L is C═O, Q is absent, and R⁷ is H.

In certain embodiments, R⁶ is C₁₋₆alkyl, R⁷ is C₁₋₆alkyl, Q is absent, and L is C═O. In certain such embodiments, R⁷ is ethyl, isopropyl, 2,2,2-trifluoroethyl, or 2-(methylsulfonyl)ethyl.

In other embodiments, L is C═O, Q is absent, and R⁷ is C₁₋₆aralkyl. In certain such embodiments, R⁷ is selected from 2-phenylethyl, phenylmethyl, (4-methoxyphenyl)methyl, (4-chlorophenyl)methyl, and (4-fluorophenyl)methyl.

In other embodiments, L is C═O, Q is absent, R⁶ is C₁₋₆alkyl, and R⁷ is aryl. In certain such embodiments, R⁷ is substituted or unsubstituted phenyl.

In certain embodiments, L is C═O, Q is absent or O, n is 0 or 1, and R⁷ is —(CH₂)ₙcarbocyclyl. In certain such embodiments, R⁷ is cyclopropyl or cyclohexyl.

In certain embodiments, L and A are C═O, Q is absent, Z is O, n is an integer from 1 to 8 (preferably 1), and R⁷ is selected from R⁸ZA-C₁₋₈alkyl-, R¹¹Z—C₁₋₈alkyl-, R⁸ZA-C₁₋₈alkyl-ZAZ-C₁₋₈alkyl-, (R⁸O)(R⁹O)P(═O)O—C₁₋₈alkyl-ZAZ-C₁₋₈ alkyl-, (R⁸O)(R⁹O)P(═O)O—C₁₋₈alkyl-Z—C₁₋₈ alkyl-, and heterocyclylMZAZ-C₁₋₈alkyl-, wherein each occurrence of A is independently other than a covalent bond. In certain such embodiments, R⁷ is heterocyclylMZAZ-C₁₋₈alkyl- where heterocyclyl is substituted or unsubstituted oxodioxolenyl or N(R¹²)(R¹³), wherein R¹² and R¹³ together are C₁₋₆alkyl-Y—C₁₋₆alkyl, preferably C₁₋₃alkyl-Y—C₁₋₃ alkyl, thereby forming a ring.

In certain preferred embodiments, L is C═O, Q is absent, n is an integer from 1 to 8, and R⁷ is selected from (R⁸O)(R⁹O)P(═O)O—C₁₋₈alkyl-, (R¹⁰)₂NC₁₋₈alkyl, (R¹⁰)₃N⁺(CH₂)ₙ—, and heterocyclyl-M-. In certain such embodiments, R⁷ is —C₁₋₈alkylN(R¹⁰)₂ or —C₁₋₈alkylN⁺(R¹⁰)₃, where R¹⁰ is C₁₋₆alkyl. In certain other such embodiments, R⁷ is heterocyclylM-, where heterocyclyl is selected from morpholino, piperidino, piperazino, and pyrrolidino.

In certain embodiments, L is C═O, R⁶ is C₁₋₆alkyl, Q is selected from O and NH and R⁷ is selected from C₁₋₆alkyl, cycloalkyl-M, C₁₋₆aralkyl, and C₁₋₆heteroaralkyl. In other embodiments, L is C═O, R⁶ is C₁₋₆alkyl, Q is selected from O and NH, and R⁷ is C₁₋₆alkyl, where C₁₋₆alkyl is selected from methyl, ethyl, and isopropyl. In further embodiments, L is C═O, R⁶ is C₁₋₆alkyl, Q is selected from O and NH and R⁷ is C₁₋₆ aralkyl, where aralkyl is phenylmethyl. In other embodiments, L is C═O, R⁶ is C₁₋₆alkyl, Q is selected from O and NH, and R⁷ is C₁₋₆heteroaralkyl, where heteroaralkyl is (4-pyridyl)methyl.

In certain embodiments, L is absent or is C═O, and R⁶ and R⁷ together are C₁₋₆alkyl-Y—C₁₋₆alkyl, C₁₋₆alkyl-ZA-C₁₋₆alkyl, or C₁₋₆alkyl-A, wherein each occurrence of Z and A is independently other than a covalent bond, thereby forming a ring. In certain preferred embodiments, L is C═O, Q and Y are absent, and R⁶ and R⁷ together are C₁₋₃alkyl-Y—C₁₋₃alkyl. In another preferred embodiment, L and Q are absent, and R⁶ and R⁷ together are C₁₋₃alkyl-Y—C₁₋₃alkyl. In another preferred embodiment, L is C═O, Q is absent, Y is selected from NH and N—C₁₋₆alkyl, and R⁶ and R⁷ together are C₁₋₃alkyl-Y—C₁₋₃alkyl. In another preferred embodiment, L is C═O, Y is absent, and R⁶ and R⁷ together are C₁₋₃alkyl-Y—C₁₋₃alkyl. In another preferred embodiment, L and A are C═O, and R⁶ and R⁷ together are C₁₋₂alkyl-ZA-C₁₋₂alkyl. In another preferred embodiment, L and A are C═O and R and R⁷ together are C₂₋₃alkyl-A.

In certain embodiments, a compound of formula I has the following stereochemistry:

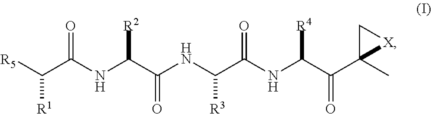

In preferred embodiments, the inhibitor has a structure of formula II or a pharmaceutically acceptable salt thereof,

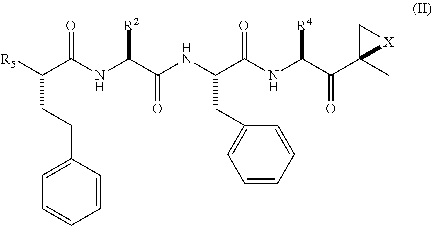

wherein each A is independently selected from C═O, C═S, and SO₂, preferably C═O; or

A is optionally a covalent bond when adjacent to an occurrence of Z;

L is absent or is selected from C═O, C═S, and SO₂, preferably L is absent or C═O;

M is absent or is C₁₋₁₂alkyl, preferably C₁₋₈alkyl;

Q is absent or is selected from O, NH, and N—C₁₋₆alkyl, preferably Q is absent, O, or NH, most preferably Q is absent or O;

X is selected from O, NH, and N—C₁₋₆alkyl, preferably O;

Y is absent or is selected from O, NH, N—C₁₋₆alkyl, S, SO, SO₂, CHOR¹⁰, and CHCO₂R¹⁰;

each Z is independently selected from O, S, NH, and N—C₁₋₆alkyl, preferably O; or

Z is optionally a covalent bond when adjacent to an occurrence of A;

R² and R⁴ are each independently selected from C₁₋₆alkyl, C₁₋₆hydroxyalkyl, C₁₋₆alkoxyalkyl, aryl, and C₁₋₆aralkyl, any of which is optionally substituted with one or more of amide, amine, carboxylic acid (or a salt thereof), ester (including C₁₋₅ alkyl ester and aryl ester), thiol, or thioether substituents;

R⁵ is N(R⁶)LQR⁷;

R⁶ is selected from hydrogen, OH, and C₁₋₆alkyl, preferably C₁₋₆alkyl;

R⁷ is selected from hydrogen, C₁₋₆alkyl, C₁₋₆alkenyl, C₁₋₆alkynyl, aryl, C₁₋₆aralkyl, heteroaryl, C₁₋₆heteroaralkyl, R⁸ZAZ-C₁₋₈alkyl-, R¹¹Z—C₁₋₈alkyl-, (R⁸O)(R⁹O)P(═O)O—C₁₋₈alkyl-ZAZ-C₁₋₈ alkyl-, R⁸ZAZ—C₁₋₈ alkyl-ZAZ-C₁₋₈alkyl-, heterocyclylMZAZ-C₁₋₈alkyl-, (R⁸O)(R⁹O)P(═O)O—C₁₋₈alkyl-, (R¹⁰)₂N—C₁₋₁₂alkyl-, (R¹⁰)₃N⁺—C₁₋₁₂alkyl-, heterocyclylM-, carbocyclylM-, R¹¹SO₂C₁₋₈alkyl-, and R¹¹SO₂NH; preferably C₁₋₆alkyl, C₁₋₆alkenyl, C₁₋₆alkynyl, aryl, C₁₋₆aralkyl, heteroaryl, C₁₋₆heteroaralkyl, R⁸ZA-C₁₋₈alkyl-, R¹¹Z—C₁₋₈alkyl-, (R⁸O)(R⁹O)P(═O)O—C₁₋₈alkyl-ZAZ-C₁₋₈ alkyl-, (R⁸O)(R⁹O)P(═O)O—C₁₋₈alkyl-Z—C₁₋₈ alkyl-, R⁸ZA-C₁₋₈alkyl-ZAZ-C₁₋₈alkyl-, heterocyclylMZAZ-C₁₋₈alkyl-, (R⁸O)(R⁹O)P(═O)O—C₁₋₈alkyl-, (R¹⁰)₂N—C₁₋₈alkyl-, (R¹⁰)₃N⁺—C₁₋₈alkyl-, heterocyclylM-, carbocyclylM-, R¹¹SO₂C₁₋₈alkyl-, and R¹¹SO₂NH, wherein each occurrence of Z and A is independently other than a covalent bond; or

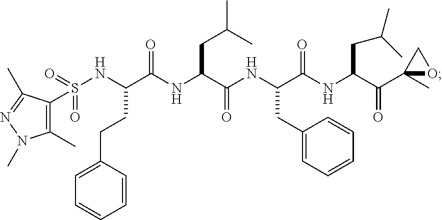 [SMILES: Cc1nn(C)c(C)c1S(=O)(=O)N[C@@H](CCc1ccccc1)C(=O)N[C@@H](CC(C)C)C(=O)N[C@@H](Cc1ccccc1)C(=O)N[C@@H](CC(C)C)C(=O)[C@@]1(C)CO1]

R⁶ and R⁷ together are C₁₋₆alkyl-Y—C₁₋₆alkyl, C₁₋₆alkyl-ZAZ-C₁₋₆alkyl, ZAZ-C₁₋₆alkyl-ZAZ-C₁₋₆alkyl, ZAZ-C₁₋₆alkyl-ZAZ, or C₁₋₆alkyl-A, thereby forming a ring; preferably C₁₋₂alkyl-Y—C₁₋₂alkyl, C₁₋₂alkyl-ZA-C₁₋₂alkyl, A-C₁₋₂alkyl-ZA-C₁₋₂alkyl, A-C₁₋₃alkyl-A, or C₁₋₄alkyl-A, wherein each occurrence of Z and A is independently other than a covalent bond;

R⁸ and R⁹ are independently selected from hydrogen, metal cation, C₁₋₆alkyl, C₁₋₆alkenyl, C₁₋₆alkynyl, aryl, heteroaryl, C₁₋₆aralkyl, and C₁₋₆heteroaralkyl, preferably from hydrogen, metal cation, and C₁₋₆alkyl, or R⁸ and R⁹ together are C₁₋₆alkyl, thereby forming a ring;

each R¹⁰ is independently selected from hydrogen and C₁₋₆alkyl, preferably C₁₋₆alkyl; and

R¹¹ is independently selected from hydrogen, C₁₋₆alkyl, C₁₋₆alkenyl, C₁₋₆alkynyl, carbocyclyl, heterocyclyl, aryl, heteroaryl, C₁₋₆aralkyl, and C₁₋₆heteroaralkyl,

provided that when R⁶ is H or CH₃ and Q is absent, LR⁷ is not hydrogen, unsubstituted C₁₋₆alkylC═O, a further chain of amino acids, t-butoxycarbonyl (Boc), benzoyl (Bz), fluoren-9-ylmethoxycarbonyl (Fmoc), triphenylmethyl(trityl), benzyloxycarbonyl (Cbz), trichloroethoxycarbonyl (Troc); or substituted or unsubstituted aryl or heteroaryl; and

in any occurrence of the sequence ZAZ, at least one member of the sequence must be other than a covalent bond.

In certain embodiments, L is C═O, Q is absent, X is O, R⁶ is H, and R² and R⁴ are selected from C₁₋₆alkyl and C₁₋₆aralkyl. In preferred such embodiments, R² and R⁴ are C₁₋₆alkyl. In the most preferred such embodiment, R² and R⁴ are isobutyl.

In certain embodiments, L is C═O, Q is absent, X is O, R⁶ is H, R² and R⁴ are isobutyl, and R⁷ is heterocyclylM-, where the heterocycle is a nitrogen-containing heterocycle, such as piperazino (including N-(lower alkyl) piperazino), morpholino, and piperidino. In preferred such embodiments, M is CH₂.

In certain embodiments, a compound of formula II is selected from:

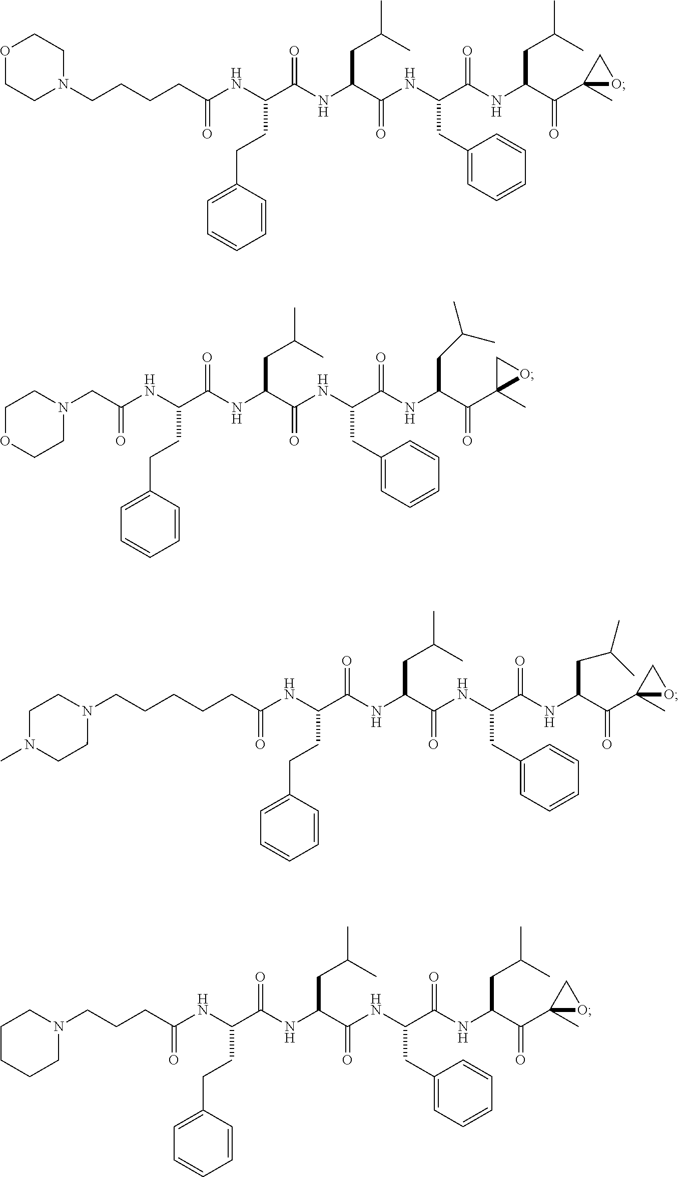

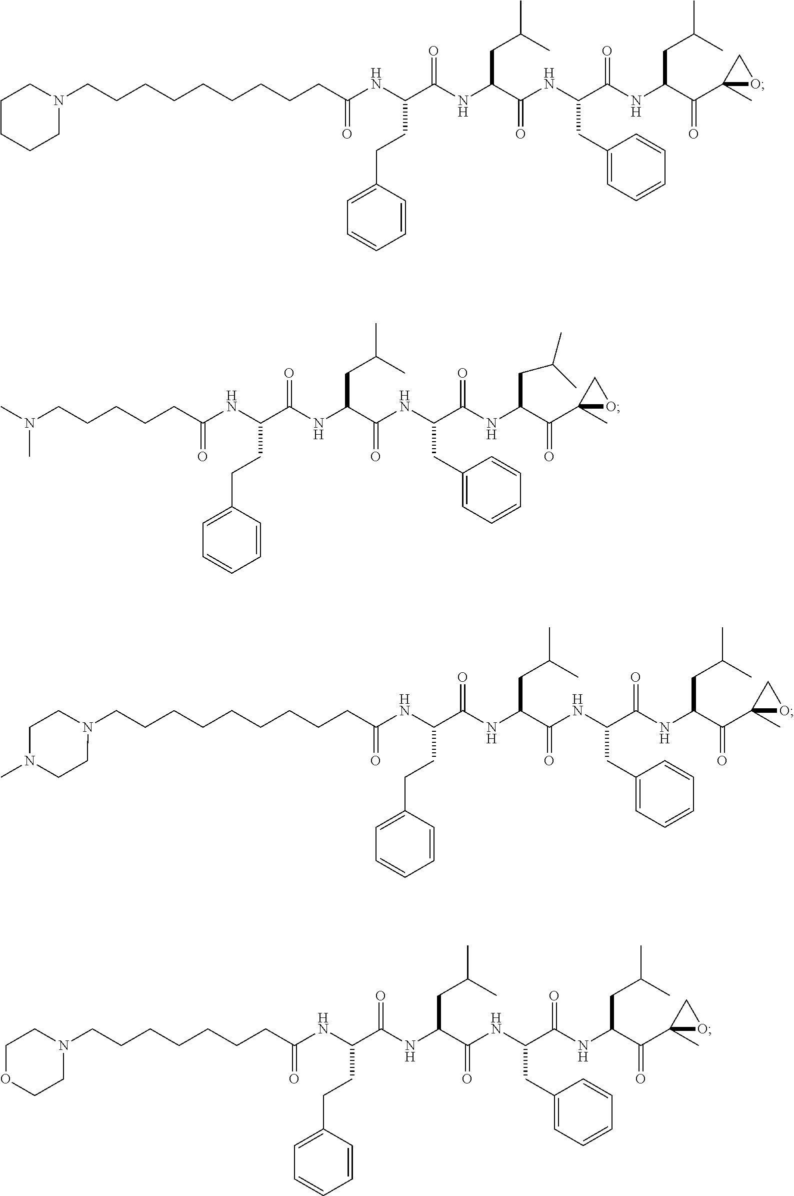 [SMILES: CC(C)C[C@H](NC(=O)[C@H](CCc1ccccc1)NC(=O)CCCCCCCCCN1CCCCC1)C(=O)N[C@@H](Cc1ccccc1)C(=O)N[C@@H](CC(C)C)C(=O)[C@@]1(C)CO1 ; CC(C)C[C@H](NC(=O)[C@H](CCc1ccccc1)NC(=O)CCCCCN(C)C)C(=O)N[C@@H](Cc1ccccc1)C(=O)N[C@@H](CC(C)C)C(=O)[C@@]1(C)CO1 ; CC(C)C[C@H](NC(=O)[C@H](CCc1ccccc1)NC(=O)CCCCCCCCCN1CCN(C)CC1)C(=O)N[C@@H](Cc1ccccc1)C(=O)N[C@@H](CC(C)C)C(=O)[C@@]1(C)CO1 ; CC(C)C[C@H](NC(=O)[C@H](CCc1ccccc1)NC(=O)CCCCCCCN1CCOCC1)C(=O)N[C@@H](Cc1ccccc1)C(=O)N[C@@H](CC(C)C)C(=O)[C@@]1(C)CO1]

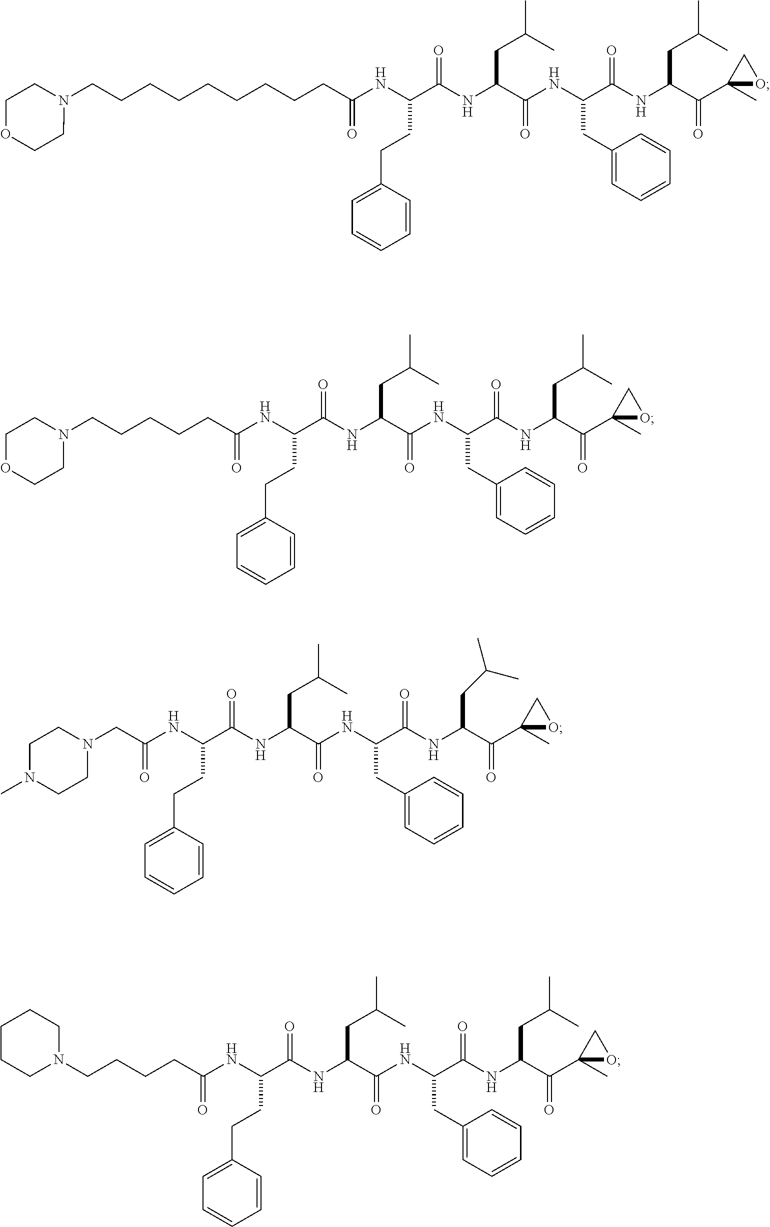 [SMILES: CC(C)C[C@H](NC(=O)[C@H](CCc1ccccc1)NC(=O)CCCCCCCCCN1CCOCC1)C(=O)N[C@@H](Cc1ccccc1)C(=O)N[C@@H](CC(C)C)C(=O)[C@@]1(C)CO1 ; CC(C)C[C@H](NC(=O)[C@H](CCc1ccccc1)NC(=O)CCCCCN1CCOCC1)C(=O)N[C@@H](Cc1ccccc1)C(=O)N[C@@H](CC(C)C)C(=O)[C@@]1(C)CO1 ; CC(C)C[C@H](NC(=O)[C@H](CCc1ccccc1)NC(=O)CN1CCN(C)CC1)C(=O)N[C@@H](Cc1ccccc1)C(=O)N[C@@H](CC(C)C)C(=O)[C@@]1(C)CO1 ; CC(C)C[C@H](NC(=O)[C@H](CCc1ccccc1)NC(=O)CCCCN1CCCCC1)C(=O)N[C@@H](Cc1ccccc1)C(=O)N[C@@H](CC(C)C)C(=O)[C@@]1(C)CO1]

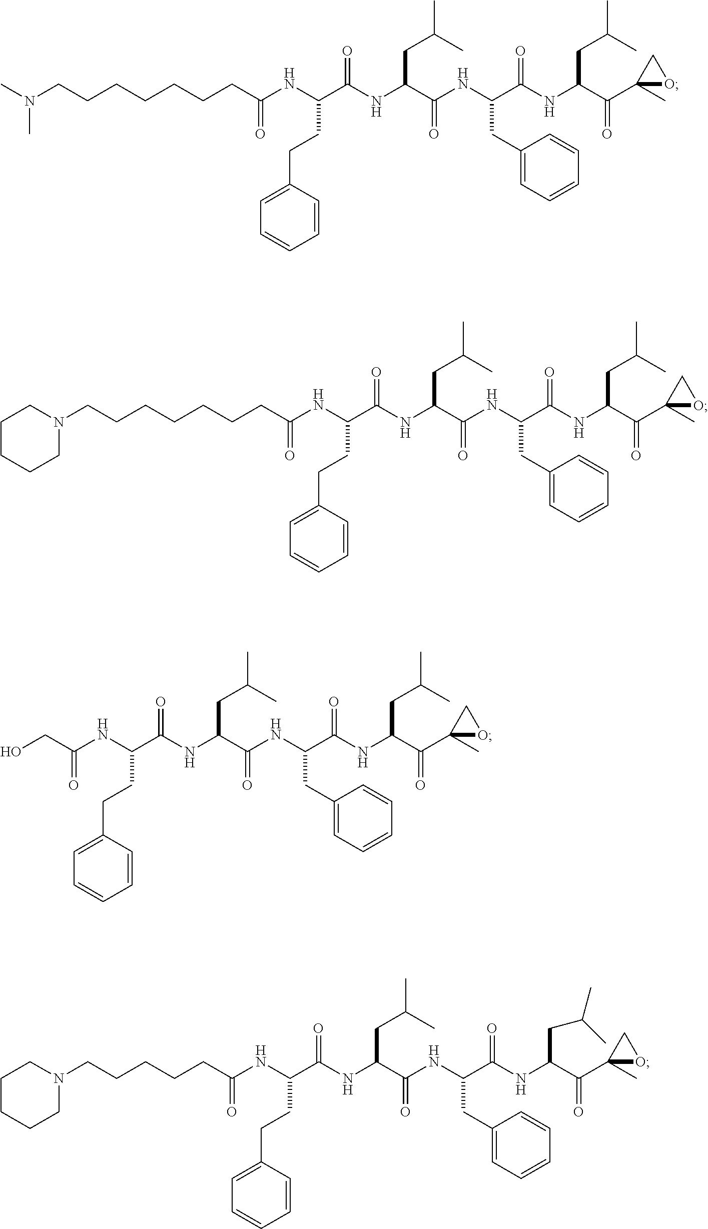 [SMILES: CC(C)C[C@H](NC(=O)[C@H](CCc1ccccc1)NC(=O)CCCCCCCN(C)C)C(=O)N[C@@H](Cc1ccccc1)C(=O)N[C@@H](CC(C)C)C(=O)[C@@]1(C)CO1 ; CC(C)C[C@H](NC(=O)[C@H](CCc1ccccc1)NC(=O)CCCCCCCN1CCCCC1)C(=O)N[C@@H](Cc1ccccc1)C(=O)N[C@@H](CC(C)C)C(=O)[C@@]1(C)CO1 ; CC(C)C[C@H](NC(=O)[C@H](CCc1ccccc1)NC(=O)CO)C(=O)N[C@@H](Cc1ccccc1)C(=O)N[C@@H](CC(C)C)C(=O)[C@@]1(C)CO1 ; CC(C)C[C@H](NC(=O)[C@H](CCc1ccccc1)NC(=O)CCCCCN1CCCCC1)C(=O)N[C@@H](Cc1ccccc1)C(=O)N[C@@H](CC(C)C)C(=O)[C@@]1(C)CO1]

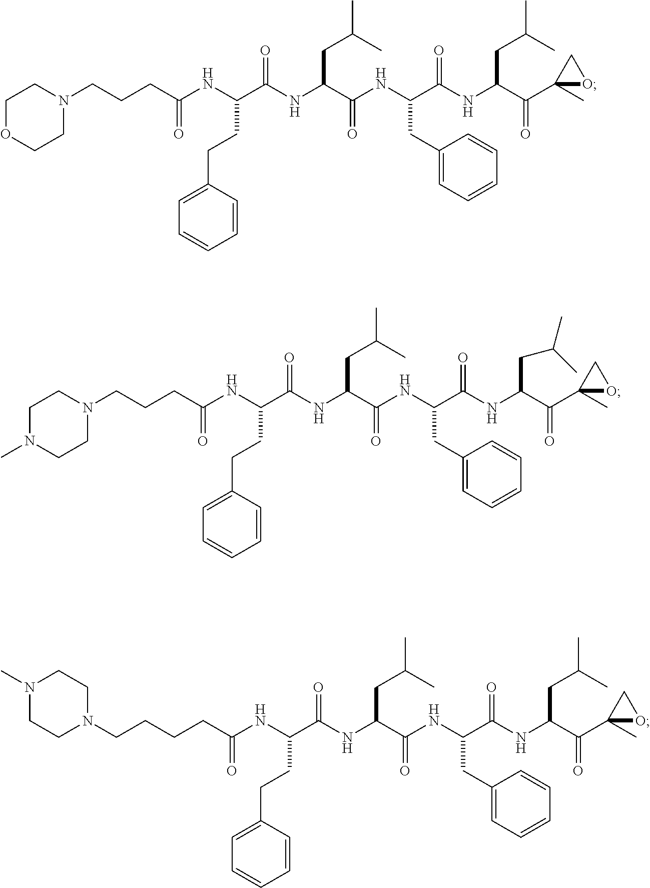 [SMILES: CC(C)C[C@H](NC(=O)[C@H](CCc1ccccc1)NC(=O)CCCN1CCOCC1)C(=O)N[C@@H](Cc1ccccc1)C(=O)N[C@@H](CC(C)C)C(=O)[C@@]1(C)CO1 ; CC(C)C[C@H](NC(=O)[C@H](CCc1ccccc1)NC(=O)CCCN1CCN(C)CC1)C(=O)N[C@@H](Cc1ccccc1)C(=O)N[C@@H](CC(C)C)C(=O)[C@@]1(C)CO1 ; CC(C)C[C@H](NC(=O)[C@H](CCc1ccccc1)NC(=O)CCCCN1CCN(C)CC1)C(=O)N[C@@H](Cc1ccccc1)C(=O)N[C@@H](CC(C)C)C(=O)[C@@]1(C)CO1]

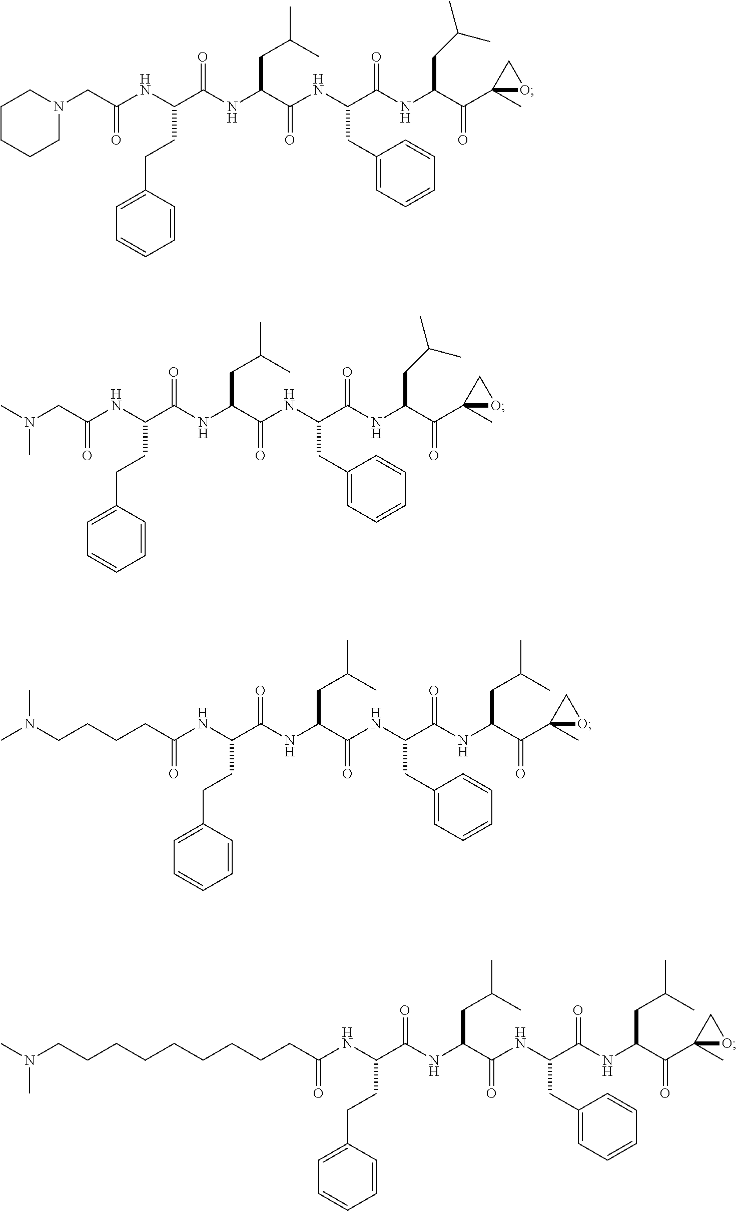 [SMILES: CC(C)C[C@H](NC(=O)[C@H](CCc1ccccc1)NC(=O)CN1CCCCC1)C(=O)N[C@@H](Cc1ccccc1)C(=O)N[C@@H](CC(C)C)C(=O)[C@@]1(C)CO1 ; CC(C)C[C@H](NC(=O)[C@H](CCc1ccccc1)NC(=O)CN(C)C)C(=O)N[C@@H](Cc1ccccc1)C(=O)N[C@@H](CC(C)C)C(=O)[C@@]1(C)CO1 ; CC(C)C[C@H](NC(=O)[C@H](CCc1ccccc1)NC(=O)CCCCN(C)C)C(=O)N[C@@H](Cc1ccccc1)C(=O)N[C@@H](CC(C)C)C(=O)[C@@]1(C)CO1 ; CC(C)C[C@H](NC(=O)[C@H](CCc1ccccc1)NC(=O)CCCCCCCCCN(C)C)C(=O)N[C@@H](Cc1ccccc1)C(=O)N[C@@H](CC(C)C)C(=O)[C@@]1(C)CO1]

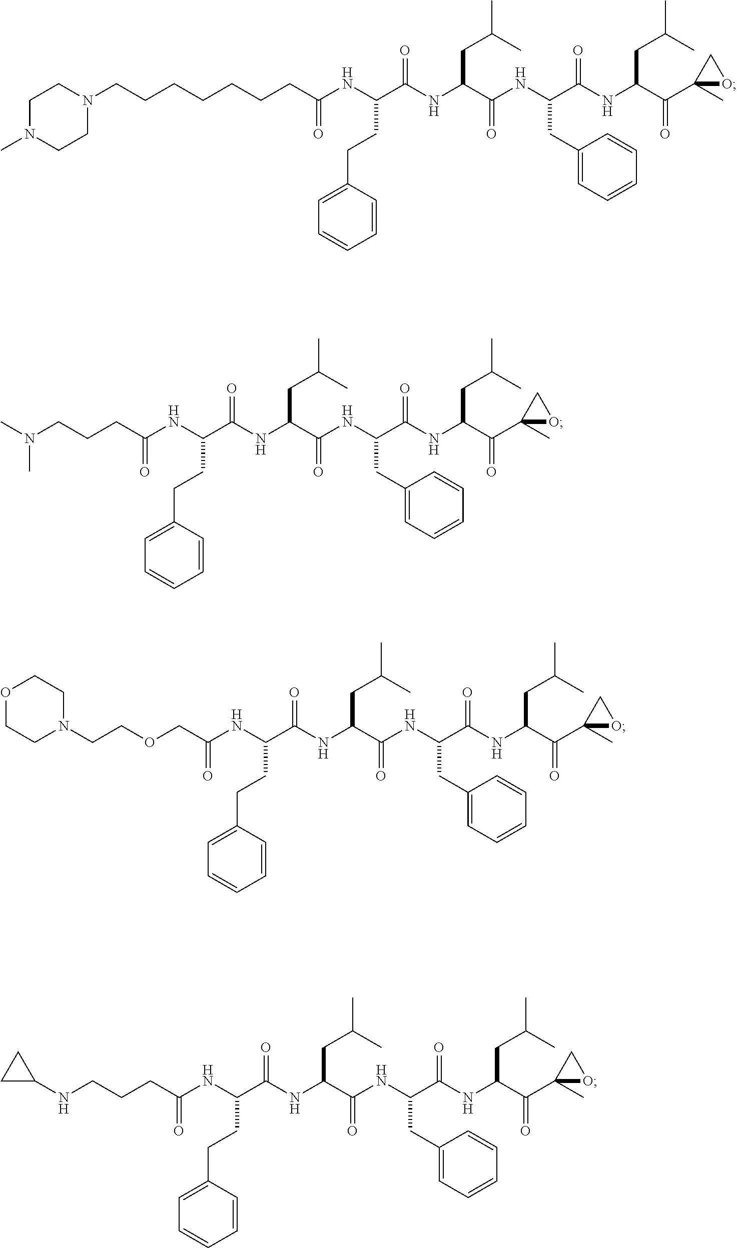 [SMILES: CC(C)C[C@H](NC(=O)[C@H](CCc1ccccc1)NC(=O)CCCCCCCN1CCN(C)CC1)C(=O)N[C@@H](Cc1ccccc1)C(=O)N[C@@H](CC(C)C)C(=O)[C@@]1(C)CO1 ; CC(C)C[C@H](NC(=O)[C@H](CCc1ccccc1)NC(=O)CCCN(C)C)C(=O)N[C@@H](Cc1ccccc1)C(=O)N[C@@H](CC(C)C)C(=O)[C@@]1(C)CO1 ; CC(C)C[C@H](NC(=O)[C@H](CCc1ccccc1)NC(=O)COCCN1CCOCC1)C(=O)N[C@@H](Cc1ccccc1)C(=O)N[C@@H](CC(C)C)C(=O)[C@@]1(C)CO1 ; CC(C)C[C@H](NC(=O)[C@H](CCc1ccccc1)NC(=O)CCCNC1CC1)C(=O)N[C@@H](Cc1ccccc1)C(=O)N[C@@H](CC(C)C)C(=O)[C@@]1(C)CO1]

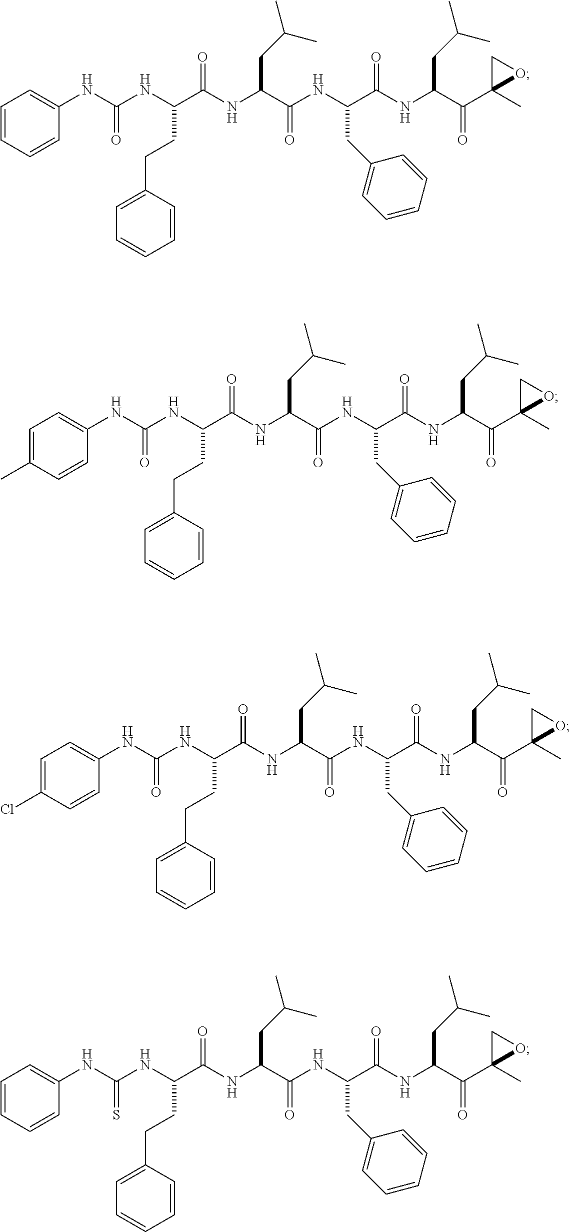 [SMILES: CC(C)C[C@H](NC(=O)[C@H](CCc1ccccc1)NC(=O)Nc1ccccc1)C(=O)N[C@@H](Cc1ccccc1)C(=O)N[C@@H](CC(C)C)C(=O)[C@@]1(C)CO1 ; Cc1ccc(NC(=O)N[C@@H](CCc2ccccc2)C(=O)N[C@@H](CC(C)C)C(=O)N[C@@H](Cc2ccccc2)C(=O)N[C@@H](CC(C)C)C(=O)[C@@]2(C)CO2)cc1 ; CC(C)C[C@H](NC(=O)[C@H](CCc1ccccc1)NC(=O)Nc1ccc(Cl)cc1)C(=O)N[C@@H](Cc1ccccc1)C(=O)N[C@@H](CC(C)C)C(=O)[C@@]1(C)CO1 ; CC(C)C[C@H](NC(=O)[C@H](CCc1ccccc1)NC(=S)Nc1ccccc1)C(=O)N[C@@H](Cc1ccccc1)C(=O)N[C@@H](CC(C)C)C(=O)[C@@]1(C)CO1]

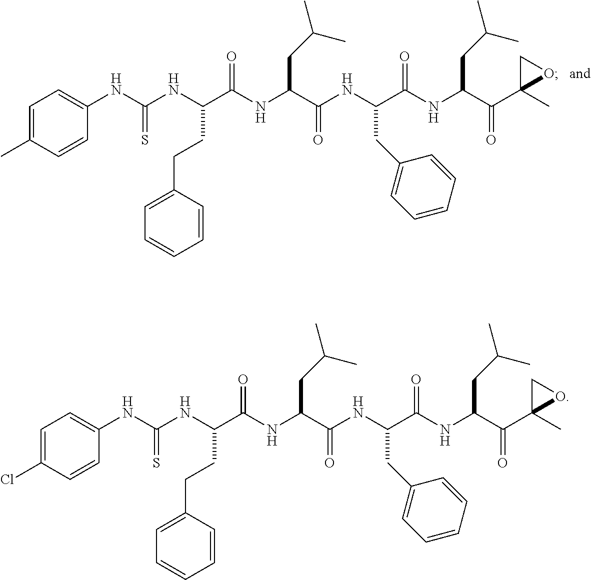 [SMILES: Cc1ccc(NC(=S)N[C@@H](CCc2ccccc2)C(=O)N[C@@H](CC(C)C)C(=O)N[C@@H](Cc2ccccc2)C(=O)N[C@@H](CC(C)C)C(=O)[C@@]2(C)CO2)cc1 ; "and" ; CC(C)C[C@H](NC(=O)[C@H](CCc1ccccc1)NC(=S)Nc1ccc(Cl)cc1)C(=O)N[C@@H](Cc1ccccc1)C(=O)N[C@@H](CC(C)C)C(=O)[C@@]1(C)CO1]

The chemical terms noted above in the definition above of “peptide epoxy ketone protease inhibitor” shall be those ascribed to the definitions thereof set forth in U.S. Pat. No. 7,232,818.

A particularly advantageous peptide epoxy ketone protease inhibitor for use herein is carfilzomib, (S)-4-methyl-N—((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoacetamido)-4-phenylbutanamido)pentanamide:

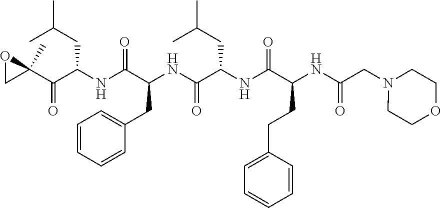 [SMILES: CC(C)C[C@H](NC(=O)[C@H](CCc1ccccc1)NC(=O)CN1CCOCC1)C(=O)N[C@@H](Cc1ccccc1)C(=O)N[C@@H](CC(C)C)C(=O)[C@@]1(C)CO1]

The term “small molecule drug conjugate that is trapped intracellularly” or “binding moiety drug conjugate that is trapped intracellularly” or “SDC-TRAP” refers to a binding moiety and peptide epoxy ketone protease inhibitor joined to one another, or acting as if joined to one another. A binding moiety and peptide epoxy ketone protease inhibitor can be joined through essentially any chemical or physical force, either directly (e.g., binding moiety and peptide epoxy ketone protease inhibitor viewed as two moieties on the same molecule, or a single moiety having both functions) or through an intermediate (e.g., linker). For example, a binding moiety and peptide epoxy ketone protease inhibitor can be joined by one or more covalent bonds, ionic bonds, hydrogen bonds, the hydrophobic effect, dipole-dipole forces, ion-dipole forces, dipole-induced dipole forces, instantaneous dipole-induced dipole forces, and/or combinations thereof. Preferentially, the SDC-TRAP will be capable of passive and/or active transport into a cell comprising a target. Moreover, SDC-TRAP molecules of the invention may comprise multiple peptide epoxy ketone protease inhibitors conjugated to the binding moiety.

The term “linker” or “linking moiety,” as used herein in the context of binding moiety, effector moieties, and/or SDC-TRAPs refers to a chemical moiety that joins two other moieties (e.g., a binding moiety and a peptide epoxy ketone protease inhibitor). A linker can covalently join a binding moiety and a peptide epoxy ketone protease inhibitor. A linker can include a cleavable linker, for example an enzymatically cleavable linker. A linker can include a disulfide, carbamate, amide, ester, and/or ether linkers.

As used herein, a “ligand” is a substance (e.g., a binding moiety) that can form a complex with a biomolecule. The ligand and/or formation of the ligand-biomolecule complex can have a biological or chemical effect, such as a therapeutic effect, cytotoxic effect, and/or imaging effect.

As used herein, a “prodrug” is a pharmacological substance that is administered in an inactive or less than fully active form and that is subsequently converted to an active pharmacological agent (i.e., the drug) through a metabolic processes. Prodrugs can be used to improve how the intended drug is absorbed, distributed, metabolized, and/or excreted. A prodrug may also be used to improve how selectively the intended drug interacts with cells or processes that are not its intended target (e.g., to reduce adverse or unintended effects of the intended drug, for example a chemotherapy drug).

The phrase “Hsp90 ligand or a prodrug thereof” refers generally to molecules that bind to and in some cases effect Hsp90, and inactive forms (i.e., prodrugs) thereof. An Hsp90 ligand can be an “Hsp90 inhibitor,” which is understood as a therapeutic agent that reduces the activity of Hsp90 either by directly interacting with Hsp90 or by, for example, preventing the formation of the Hsp90/CDC37 complex such that the expression and proper folding of at least one client protein of Hsp90 is inhibited. “Hsp90” includes each member of the family of heat shock proteins having a mass of about 90-kilodaltons. For example, in humans the highly conserved Hsp90 family includes cytosolic Hsp90a and Hsp90P isoforms, as well as GRP94, which is found in the endoplasmic reticulum, and HSP75/TRAP1, which is found in the mitochondrial matrix. As used herein, Hsp90 inhibitors include, but are not limited to ganetespib, geldanamycin (tanespimycin), e.g., IPI-493, macbecins, tripterins, tanespimycins, e.g., 17-AAG (alvespimycin), KF-55823, radicicols, KF-58333, KF-58332, 17-DMAG, IPI-504, BIIB-021, BIIB-028, PU-H64, PU-H71, PU-DZ8, PU-HZ151, SNX-2112, SNX-2321, SNX-5422, SNX-7081, SNX-8891, SNX-0723, SAR-567530, ABI-287, ABI-328, AT-13387, NSC-113497, PF-3823863, PF-4470296, EC-102, EC-154, ARQ-250-RP, BC-274, VER-50589, KW-2478, BHI-001, AUY-922, EMD-614684, EMD-683671, XL-888, VER-51047, KOS-2484, KOS-2539, CUDC-305, MPC-3100, CH-5164840, PU-DZ13, PU-HZ151, PU-DZ13, VER-82576, VER-82160, VER-82576, VER-82160, NXD-30001, NVP-HSP990, SST-0201CL1, SST-0115AA1, SST-0221AA1, SST-0223AA1, novobiocin (a C-terminal Hsp90i, herbinmycin A, radicicol, CCT018059, PU-H71, or celastrol.

The term “therapeutic moiety” refers to molecule, compound, or fragment thereof that is used for the treatment of a disease or for improving the well-being of an organism or that otherwise exhibit healing power (e.g., pharmaceuticals, drugs, and the like). A therapeutic moiety can be a chemical, or fragment thereof, of natural or synthetic origin used for its specific action against disease, for example cancer. Therapeutic agents used for treating cancer may be called chemotherapeutic agents. As described herein, a therapeutic moiety is preferentially a small molecule. Exemplary small molecule therapeutics include those that are less than 800 Daltons, 700 Daltons, 600 Daltons, 500 Daltons, 400 Daltons, or 300 Daltons.

The term “cytotoxic moiety” refers to molecule, compound, or fragment thereof that has a toxic or poisonous effect on cells, or that kills cells. Chemotherapy and radiotherapy are forms of cytotoxic therapy. Treating cells with a cytotoxic moiety can produce a variety of results—cells may undergo necrosis, stop actively growing and dividing, or activate a genetic program of controlled cell death (i.e., apoptosis). Examples of cytotoxic moieties include the peptide epoxy ketone protease inhibitors described herein, or fragment(s) thereof.

The term “imaging moiety” refers to a molecule, compound, or fragment thereof that facilitates a technique and/or process used to create images or take measurements of a cell, tissue, and/or organism (or parts or functions thereof) for clinical and/or research purposes. An imaging moiety can produce, for example, a signal through emission and/or interaction with electromagnetic, nuclear, and/or mechanical (e.g., acoustic as in ultrasound) energy. An imaging moiety can be used, for example, in various radiology, nuclear medicine, endoscopy, thermography, photography, spectroscopy, and microscopy methods.

“Pharmaceutical conjugate” refers to a non-naturally occurring molecule that includes a binding moiety (e.g., an Hsp90-targeting moiety) associated with a peptide epoxy ketone protease inhibitor, where these two components may also be covalently bonded to each other either directly or through a linking group.

The term “drug” refers to any active agent that affects any biological process. Active agents that are considered drugs for purposes of this application are agents that exhibit a pharmacological activity. Examples of drugs include active agents that are used in the prevention, diagnosis, alleviation, treatment or cure of a disease condition.

By “pharmacologic activity” is meant an activity that modulates or alters a biological process so as to result in a phenotypic change, e.g., cell death, cell proliferation etc.

By “pharmacokinetic property” is meant a parameter that describes the disposition of an active agent in an organism or host.

By “half-life” is meant the time for one-half of an administered drug to be eliminated through biological processes, e.g., metabolism, excretion, etc.

The term “efficacy” refers to the effectiveness of a particular active agent for its intended purpose, i.e., the ability of a given active agent to cause its desired pharmacologic effect.

Once SDC-TRAP molecules of the invention enter a target cell the peptide epoxy ketone protease inhibitor is released from the SDC-TRAP. In one embodiment, the peptide epoxy ketone protease inhibitor has no activity until it is released from the SDC-TRAP. Accordingly, once the SDC-TRAP molecules enter a target cell an equilibrium exists between free and bound SDC-TRAP molecules. In one embodiment, the peptide epoxy ketone protease inhibitor is only released from the SDC-TRAP when the SDC-TRAP is not associated with the target protein. For example, when an SDC-TRAP molecule is not bound intracellular enzymes can access the linker region thereby freeing the peptide epoxy ketone protease inhibitor. Alternatively, when free SDC-TRAP molecules may be able to release peptide epoxy ketone protease inhibitors through, for example, hydrolysis of the bond or linker that connects the binding moiety and peptide epoxy ketone protease inhibitor.

Accordingly, the rate of peptide epoxy ketone protease inhibitor release and the amount of peptide epoxy ketone protease inhibitor released can be controlled by using binding moieties that bind to the target protein with different affinities. For example, binding moieties that bind to the target protein with lower affinity will be free, resulting in higher concentrations of unbound intracellular SDC-TRAP, and thereby resulting in higher concentrations of free peptide epoxy ketone protease inhibitor. Therefore, in at least one embodiment, irreversibly-binding moieties are incompatible with certain aspects of the invention, e.g., those embodiments where peptide epoxy ketone protease inhibitor release is based on free intracellular SDC-TRAP molecules.

In one embodiment, SDC-TRAPs have favorable safety profiles, for example, when compared to, for example, the binding moiety or peptide epoxy ketone protease inhibitor alone. One reason for the increased safety profile is the rapid clearance of SDC-TRAP molecules that do not enter into a target cell.

A number of exemplary SDC-TRAP molecules are set forth in the examples.

Binding Moieties

A primary role of a binding moiety is to ensure that the SDC-TRAP delivers its payload—the peptide epoxy ketone protease inhibitor—to its target by binding to a molecular target in or on a target cell or tissue. In this respect, it is not necessary that the binding moiety also have an effect on the target (e.g., in the case of an Hsp90-targeting moiety, to inhibit Hsp90 in the manner that Hsp90 inhibitors are known to do, that is, exhibit pharmacological activity or interfere with its function), but in some embodiments, the binding moiety does have an effect on the target. Accordingly, in various embodiments, an activity of the SDC-TRAP is due solely to the peptide epoxy ketone protease inhibitor exerting a pharmacological effect on the target cell(s), which has been better facilitated by the pharmaceutical conjugate targeting the target cell(s). In other embodiments, an activity of the SDC-TRAP is due in part to the binding moiety—that is, the binding moiety can have an effect beyond targeting.

The molecular target of a binding moiety may or may not be part of a complex or structure of a plurality of biological molecules, e.g., lipids, where the complexes or structures may include lipoproteins, lipid bilayers, and the like. However, in many embodiments, the molecular target to which the binding moiety binds will be free (e.g., cytoplasmic globular protein and/or not be part of a macromolecular assembly or aggregation). The present invention can exploit the selectively high presence of a molecular target in locations of high physiological activity (e.g., Hsp90 in oncological processes). For example, where a drug target is an intracellular drug target, a corresponding molecular target (e.g., Hsp90) can be present in the cell. Likewise, where a drug target is an extracellular drug target, a corresponding molecular target (e.g., Hsp90) can be extracellular, proximal, or associated with the extracellular cell membrane of the target cell or tissue.

In various embodiments, a binding moiety can effect a target cell or tissue (e.g., in the case of an Hsp90-targeting moiety that in fact inhibits Hsp90, for example, Hsp90i). In such embodiments, a pharmacological activity of the binding moiety contributes to, complements, or augments, the pharmacological activity of the peptide epoxy ketone protease inhibitor. Such embodiments go beyond the advantages combination therapies (e.g., a cancer combination therapy of Hsp90i and a second drug such as ganetespib or crizotinib) by providing a therapy that can be carried out by administration of a single SDC-TRAP that realizes both the benefits of the combination therapy and targeting.

In various illustrative embodiments the binding moiety can be an Hsp90-targeting moiety, for example a triazole/resorcinol-based compound that binds Hsp90, or a resorcinol amide-based compound that binds Hsp90, e.g., ganetespib, AUY-922 or AT-13387. In another embodiment, the binding moiety may advantageously be an Hsp90-binding compound of formula (I):

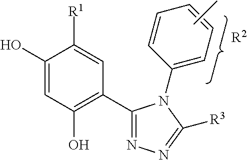

wherein

R¹ may be alkyl, aryl, halide, carboxamide or sulfonamide; R² may be alkyl, cycloalkyl, aryl or heteroaryl, wherein when R² is a 6 membered aryl or heteroaryl, R² is substituted at the 3- and 4-positions relative to the connection point on the triazole ring, through which a linker L is attached; and R³ may be SH, OH, —CONHR⁴, aryl or heteroaryl, wherein when R³ is a 6 membered aryl or heteroaryl, R³ is substituted at the 3 or 4 position.

In another embodiment, the binding moiety may advantageously be an Hsp90-binding compound of formula (II):

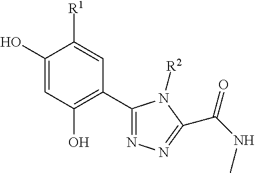

wherein

R¹ may be alkyl, aryl, halo, carboxamido, sulfonamido; and R² may be optionally substituted alkyl, cycloalkyl, aryl or heteroaryl. Examples of such compounds include 5-(2,4-dihydroxy-5-isopropylphenyl)-N-(2-morpholinoethyl)-4-(4-(morpholinomethyl)phenyl)-4H-1,2,4-triazole-3-carboxamide and 5-(2,4-dihydroxy-5-isopropylphenyl)-4-(4-(4-methylpiperazin-1-yl)phenyl)-N-(2,2,2-trifluoroethyl)-4H-1,2,4-triazole-3-carboxamide.

In another embodiment, the binding moiety may advantageously be an Hsp90-binding compound of formula (III):

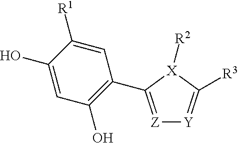

wherein

X, Y, and Z may independently be CH, N, O or S (with appropriate substitutions and satisfying the valency of the corresponding atoms and aromaticity of the ring); R¹ may be alkyl, aryl, halide, carboxamido or sulfonamido; R² may be substituted alkyl, cycloalkyl, aryl or heteroaryl, where a linker L is connected directly or to the extended substitutions on these rings; R³ may be SH, OH, NR⁴R⁵ AND —CONHR⁶, to which a peptide epoxy ketone protease inhibitor may be connected; R⁴ and R⁵ may independently be H, alkyl, aryl, or heteroaryl; and R⁶ may be alkyl, aryl, or heteroaryl, having a minimum of one functional group to which a peptide epoxy ketone protease inhibitor may be connected. Examples of such compounds include AUY-922:

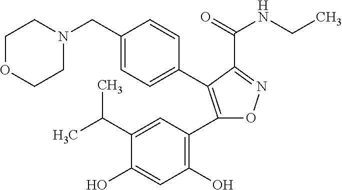 [SMILES: CCNC(=O)c1noc(-c2cc(C(C)C)c(O)cc2O)c1-c1ccc(CN2CCOCC2)cc1]

In another embodiment, the binding moiety may advantageously be an Hsp90-binding compound of formula (IV):

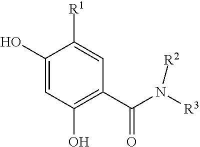

wherein

R¹ may be alkyl, aryl, halo, carboxamido or sulfonamido; R² and R³ are independently C₁-C₅ hydrocarbyl groups optionally substituted with one or more of hydroxy, halogen, C₁-C₂ alkoxy, amino, mono- and di-C₁-C₂ alkylamino; 5- to 12-membered aryl or heteroaryl groups; or, R² and R³, taken together with the nitrogen atom to which they are attached, form a 4- to 8-membered monocyclic heterocyclic group, of which up to 5 ring members are selected from O, N and S. Examples of such compounds include AT-13387:

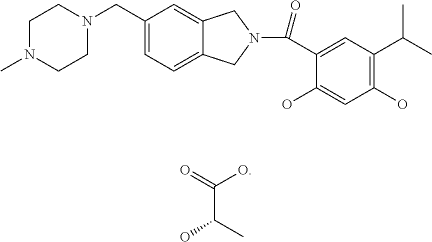

In certain embodiments, to enhance the bioavailability or delivery of the pharmaceutical conjugate, the binding moiety may be a prodrug of the Hsp90-binding compound.

Specific examples of suitable Hsp90-targeting moieties include geldanamycins, e.g., IPI-493, macbecins, tripterins, tanespimycins, e.g., 17-AAGKF-55823, radicicols, KF-58333, KF-58332, 17-DMAG, IPI-504, BIIB-021, BIIB-028, PU-H64, PU-H71, PU-DZ8, PU-HZ151, SNX-2112, SNX-2321, SNX-5422, SNX-7081, SNX-8891, SNX-0723, SAR-567530, ABI-287, ABI-328, AT-13387, NSC-113497, PF-3823863, PF-4470296, EC-102, EC-154, ARQ-250-RP, BC-274, VER-50589, KW-2478, BHI-001, AUY-922, EMD-614684, EMD-683671, XL-888, VER-51047, KOS-2484, KOS-2539, CUDC-305, MPC-3100, CH-5164840, PU-DZ13, PU-HZ151, PU-DZ13, VER-82576, VER-82160, VER-82576, VER-82160, NXD-30001, NVP-HSP990, SST-0201CL1, SST-0115AA1, SST-0221AA1, SST-0223AA1, and novobiocin (a C-terminal Hsp90i.) The selection of other Hsp90-targeting moieties will be within the grasp of one of ordinary skill in the aroom temperature. Likewise, the selection of binding moieties suitable for other molecular targets and/or other applications will be within the ability of one of ordinary skill in the aroom temperature.

Peptide Epoxy Ketone Protease Inhibitors

Carfilzomib (Kyprolis®) is a selective proteasome inhibitor approved by the FDA in 2012 for the treatment of relapsed or refractory multiple myeloma. Carfilzomib (and like compounds) is a tetrapeptide bearing an epoxy-ketone group that irreversibly inhibits the chyomotrypsin-like activity of the proteasome. It is, however, known to cause hepatotoxicity, thrombocytopenia, pulmonary complications, pulmonary hypertension, cardiac adverse reactions including heart failure and ischemia, tumor lysis syndrome, and infusion reactions (see, Kyprolis package insert]) Development of these events may require dose reductions or discontinuation of therapy, which make this drug eminently suitable for use in the SDC-TRAP conjugates described herein.

Carfilzomib is an analogue of epoxomicin and eponemycin, related natural products initially shown to inhibit tumors in animals and later to specifically inhibit the chymotrypsin-like activity of the 20S proteasome (see, e.g., J. Antibiotics (Tokyo), 1992, 45, page 1746-1752; Bioorg. Med. Chem. Let., 1999, 9, page 3335-3340; Cancer Research, 1999, 59, page 2798-2801; PNAS USA, 1999, 96, page 10403-10408; JACS, 2000, 122, 1237-1238). Carfilzomib (and like compounds) forms an irreversible dual covalent bond with the catalytic β5 subunit of the proteasome (Drug Metabolism and Disposition, 2011, 39, 1873-1882) via a two-step process: attack on the ketone group (at left, above) first, then opening the epoxide ring to form a morpholine adduct between the proteasome and the drug (Chemical Reviews, 2007, 107, 687-717).

(c1) Electron density map of epoxomicin bound to subunit β5. (c2) Schematic representation of the proposed morpholino derivative adduct formation mechanism. Binding of epoxomicin to the proteasome results in formation of a morpholino adduct between the epoxyketone pharmacophore and the active site amino terminal Thr1. Nucleophilic attack by Thr1Oγ on epoxomicin results in hemiacetal formation followed by subsequent cyclization of Thr1N onto the epoxide, resulting in an inversion of C2 and formation of the morpholino adduct. (copied from: Chemical Reviews, 2007, 107, 687-717)

In one embodiment, SDC-TRAP molecules include a binding moiety which is an Hsp90 binding compound which is desirably covalently linked to a non-reactive moiety on the peptide epoxy ketone molecule, e.g., the cyclic or heterocyclic ring on the end of the molecule. Advantageously, the linker may be a carbamate or ester linkage, as shown particularly in the Examples below.

In another embodiment, a linker on the ketone (or conversion of the ketone group as detailed herein) proximate to the epoxide group may be employed to join the binding moiety (e.g., an Hsp90 binding compound) As mentioned above, the ketone group is necessary for the initial adduct formation with the proteasome and elicit proteasome chymotrypsin-like inhibition. By masking the ketone with an oxime unit, the peptide epoxy ketone protease inhibitor, e.g., carfilzomib, would be muted, since the oxime functional group is less electrophilic than the ketone. Upon enzymatic hydrolysis of the oxime unit (e.g., inside the target cells), active peptide epoxy ketone protease inhibitor, e.g., carfilzomib would be released.

The oxime may be of varying strength, e.g., alkyl oxime, acyl oxime, carbonate oxime, carbamate oximes. The anticipated (i.e., increasing stability from left to right)

The anticipated order of stability is shown below:

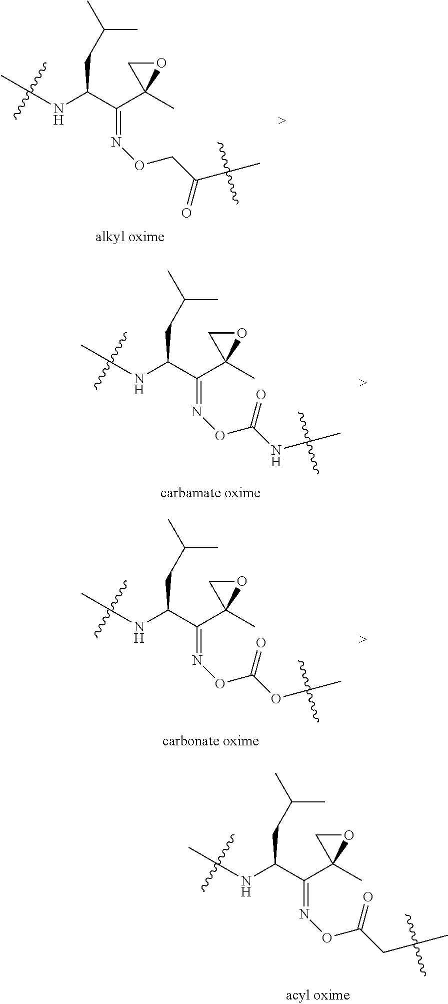 [SMILES: *N[C@@H](CC(C)C)/C(=N/OCC(*)=O)[C@@]1(C)CO1 ; *NC(=O)O/N=C(/[C@H](CC(C)C)N*)[C@@]1(C)CO1 ; *N[C@@H](CC(C)C)/C(=N/OC(=O)O*)[C@@]1(C)CO1 ; *CC(=O)O/N=C(/[C@H](CC(C)C)N*)[C@@]1(C)CO1]

Upon enzymatic hydrolysis of the modified oximes, the resulting oxime functional group, such as shown below as structure A, is more labile, resulting in the release of active peptide epoxy ketone protease inhibitor, e.g., carfilzomib. The relative strength/lability of the linker may be desirably modified by altering the degree of sterics on the linkers themselves, e.g., via incorporation of either geminal methyl, tert-butyl, cyclohexyl groups or any other large alkyl groups.

In another embodiment, the epoxide group may be modified with a sulfonate group to attach the Hsp90i binding moiety such as shown below:

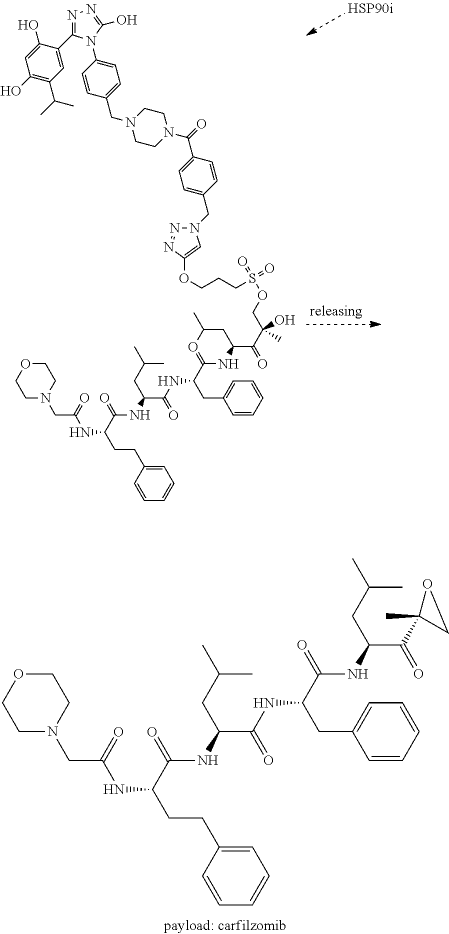

Exemplary synthetic protocols for such SDC-TRAP/peptide epoxy ketone protease inhibitor compounds are set forth in the Examples below.

Conjugation and Linking Moieties

Binding moieties and effector moieties of the present invention can be conjugated, for example, through a linker or linking moiety L, where L may be either a bond or a linking group. For example, in various embodiments, a binding moiety and a peptide epoxy ketone protease inhibitor are bound directly or are parts of a single molecule. Alternatively, a linking moiety can provide a covalent attachment between a binding moiety and peptide epoxy ketone protease inhibitor. A linking moiety, as with a direct bond, can achieve a desired structural relationship between a binding moiety and peptide epoxy ketone protease inhibitor and or an SDC-TRAP and its molecular target. A linking moiety can be inert, for example, with respect to the targeting of a binding moiety and biological activity of a peptide epoxy ketone protease inhibitor.

Appropriate linking moieties can be identified using the affinity, specificity, and/or selectivity assays described herein. Linking moieties can be selected based on size, for example, to provide an SDC-TRAP with size characteristics as described above. In various embodiments, a linking moiety can be selected, or derived from, known chemical linkers. Linking moieties can comprise a spacer group terminated at either end with a reactive functionality capable of covalently bonding to the drug or ligand moieties. Spacer groups of interest include aliphatic and unsaturated hydrocarbon chains, spacers containing heteroatoms such as oxygen (ethers such as polyethylene glycol) or nitrogen (polyamines), peptides, carbohydrates, cyclic or acyclic systems that may possibly contain heteroatoms. Spacer groups may also be comprised of ligands that bind to metals such that the presence of a metal ion coordinates two or more ligands to form a complex. Specific spacer elements include: 1,4-diaminohexane, xylylenediamine, terephthalic acid, 3,6-dioxaoctanedioic acid, ethylenediamine-N,N-diacetic acid, 1,1′-ethylenebis(5-oxo-3-pyrrolidinecarboxylic acid), 4,4′-ethylenedipiperidine. Potential reactive functionalities include nucleophilic functional groups (amines, alcohols, thiols, hydrazides), electrophilic functional groups (aldehydes, esters, vinyl ketones, epoxides, isocyanates, maleimides), functional groups capable of cycloaddition reactions, forming disulfide bonds, or binding to metals. Specific examples include primary and secondary amines, hydroxamic acids, N-hydroxysuccinimidyl esters, N-hydroxysuccinimidyl carbonates, oxycarbonylimidazoles, nitrophenylesters, trifluoroethyl esters, glycidyl ethers, vinylsulfones, and maleimides. Specific linking moieties that may find use in the SDC-TRAPs include disulfides and stable thioether moieties.

In various embodiments, a linking moiety is cleavable, for example enzymatically cleavable. A cleavable linker can be used to release a peptide epoxy ketone protease inhibitor inside a target cell after the SDC-TRAP is internalized. The susceptibility of a linking moiety to cleavage can be used to control delivery of a peptide epoxy ketone protease inhibitor. For example, a linking moiety can be selected to provide extended or prolonged release of a peptide epoxy ketone protease inhibitor in a target cell over time (e.g., a carbamate linking moiety may be subject to enzymatic cleavage by a carboxylesterase via the same cellular process used to cleave other carbamate prodrugs like capecitabine or irinotecan). In these, and various other embodiments, a linking moiety can exhibit sufficient stability to ensure good target specificity and low systemic toxicity, but not so much stability that it results in lowering the potency and efficacy of the SDC-TRAP.

Methods of Making Pharmaceutical Conjugates

A number of exemplary methods for preparing SDC-TRAP molecules are set forth in the examples. As one of skill in the art will understand, the exemplary methods set forth in the examples can be modified to make other SDC-TRAP molecules.

Methods of Use, Pharmaceutical Preparations, and Kits

The pharmaceutical conjugates find use in treatment of a host condition, e.g., a disease condition. In these methods, an effective amount of the pharmaceutical conjugate is administered to the host, where “effective amount” means a dosage sufficient to produce the desired result, e.g., an improvement in a disease condition or the symptoms associated therewith. In many embodiments, the amount of drug in the form of the pharmaceutical conjugate that need be administered to the host in order to be an effective amount will vary from that which must be administered in free drug form. The difference in amounts may vary, and in many embodiments may range from two-fold to ten-fold. In certain embodiments, e.g., where the resultant modulated pharmacokinetic property or properties result(s) in enhanced activity as compared to the free drug control, the amount of drug that is an effective amount is less than the amount of corresponding free drug that needs to be administered, where the amount may be two-fold, usually about four-fold and more usually about ten-fold less than the amount of free drug that is administered.

The pharmaceutical conjugate may be administered to the host using any convenient means capable of producing the desired result. Thus, the pharmaceutical conjugate can be incorporated into a variety of formulations for therapeutic administration. More particularly, the pharmaceutical conjugate of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. As such, administration of the pharmaceutical conjugate can be achieved in various ways, including oral, buccal, rectal, parenteral, intraperitoneal, intradermal, transdermal, intracheal, etc., administration. In pharmaceutical dosage forms, the pharmaceutical conjugate may be administered alone or in combination with other pharmaceutically active compounds.

The specific disease conditions treatable by with the subject bifunctional compounds are as varied as the types of drug moieties that can be present in the pharmaceutical conjugate. Thus, disease conditions include cellular proliferative diseases, such as neoplastic diseases, autoimmune diseases, central nervous system or neurodegenerative diseases, cardiovascular diseases, hormonal abnormality diseases, infectious diseases, and the like.

By treatment is meant at least an amelioration of the symptoms associated with the disease condition afflicting the host, where amelioration is used in a broad sense to refer to at least a reduction in the magnitude of a parameter, e.g., symptom, associated with the pathological condition being treated, such as inflammation and pain associated therewith. As such, treatment also includes situations where the pathological condition, or at least symptoms associated therewith, are completely inhibited, e.g., prevented from happening, or stopped, e.g., terminated, such that the host no longer suffers from the pathological condition, or at least the symptoms that characterize the pathological condition.

Methods of use of the invention extend beyond strict treatment of a disease. For example, the invention includes uses in a clinical or research setting to diagnose a subject, select a subject for therapy, select a subject for participation in a clinical trial, monitor the progression of a disease, monitor the effect of therapy, to determine if a subject should discontinue or continue therapy, to determine if a subject has reached a clinical end point, and to determine recurrence of a disease. The invention also includes uses in conducting research to identify effective interacting moieties and/or effector moieties and/or combinations thereof, to identify effective dosing and dose scheduling, to identify effective routes of administration, and to identify suitable targets (e.g., diseases susceptible to particular treatment).

A variety of hosts are treatable according to the subject methods. Generally such hosts are “mammals” or “mammalian,” where these terms are used broadly to describe organisms which are within the class Mammalia, including the orders carnivore (e.g., dogs and cats), rodentia (e.g., mice, guinea pigs, and rats), and primates (e.g., humans, chimpanzees, and monkeys). In many embodiments, the hosts will be humans.

The invention provides kits for treating a subject in need thereof comprising at least one SDC-TRAP and instruction for administering a therapeutically effective amount of the at least one SDC-TRAP to the subject, thereby treating the subject. The invention also provides kits for imaging, diagnosing, and/or selecting a subject comprising at least one SDC-TRAP and instruction for administering an effective amount of at least one SDC-TRAP to the subject, thereby imaging, diagnosing, and/or selecting the subject.

Kits with unit doses of the pharmaceutical conjugate, usually in oral or injectable doses and often in a storage stable formulation, are provided. In such kits, in addition to the containers containing the unit doses, an informational package insert describing the use and attendant benefits of the drugs in treating pathological condition of interest will be included. Preferred compounds and unit doses are those described herein above.

In one embodiment, an SDC-TRAP that is administered to a subject but does not enter a target cell is rapidly cleared from the body. In this embodiment, the SDC-TRAP that does not enter a target cell is rapidly cleared in order to reduce the toxicity due to the components of the SDC-TRAP, the degradation products of the SDC-TRAP or the SDC-TRAP molecule. Clearance rate can be determined by measuring the plasma concentration of the SDC-TRAP molecule as a function of time.

Likewise, SDC-TRAP molecules that enter non-targeted cells by passive diffusion rapidly exit the non-targeted cell or tissue and are either eliminated from the subject or proceed to enter and be retained a targeted cell or tissue. For example, an SDC-TRAP that is intended to treat tumor cells and is targeted to tumor cells that overexpress, for example, Hsp90 will accumulate selectively in tumor cells that overexpress Hsp90. Accordingly, very low levels of this exemplary SDC-TRAP will be present in non-tumor tissue such as normal lung tissue, heart, kidney, and the like. In one embodiment, the safety of the SDC-TRAP molecules of the invention can be determined by their lack of accumulation in non-targeted tissue. Conversely, the safety of the SDC-TRAP molecules of the invention can be determined by their selective accumulation in the targeted cells and/or tissue.

### EXAMPLES

The following examples, which are briefly summarized and then discussed in turn below, are offered by way of illustration and not by way of limitation.

Example 1 presents the synthesis of exemplary SDC-TRAPs with peptide epoxy ketone protease inhibitor effector moieties.

Example 2 presents an exemplary assay for selecting binding moieties.

Example 3 presents methods for assessing the cytotoxicity of SDC-TRAPs.

### Example 1

Exemplary SDC-TRAPs with peptide epoxy ketone protease inhibitor effector moieties may be prepared as described below.

Masking method, e.g., by modifying the ketone or epoxide group to enable attachment of an Hsp90i binding moiety:

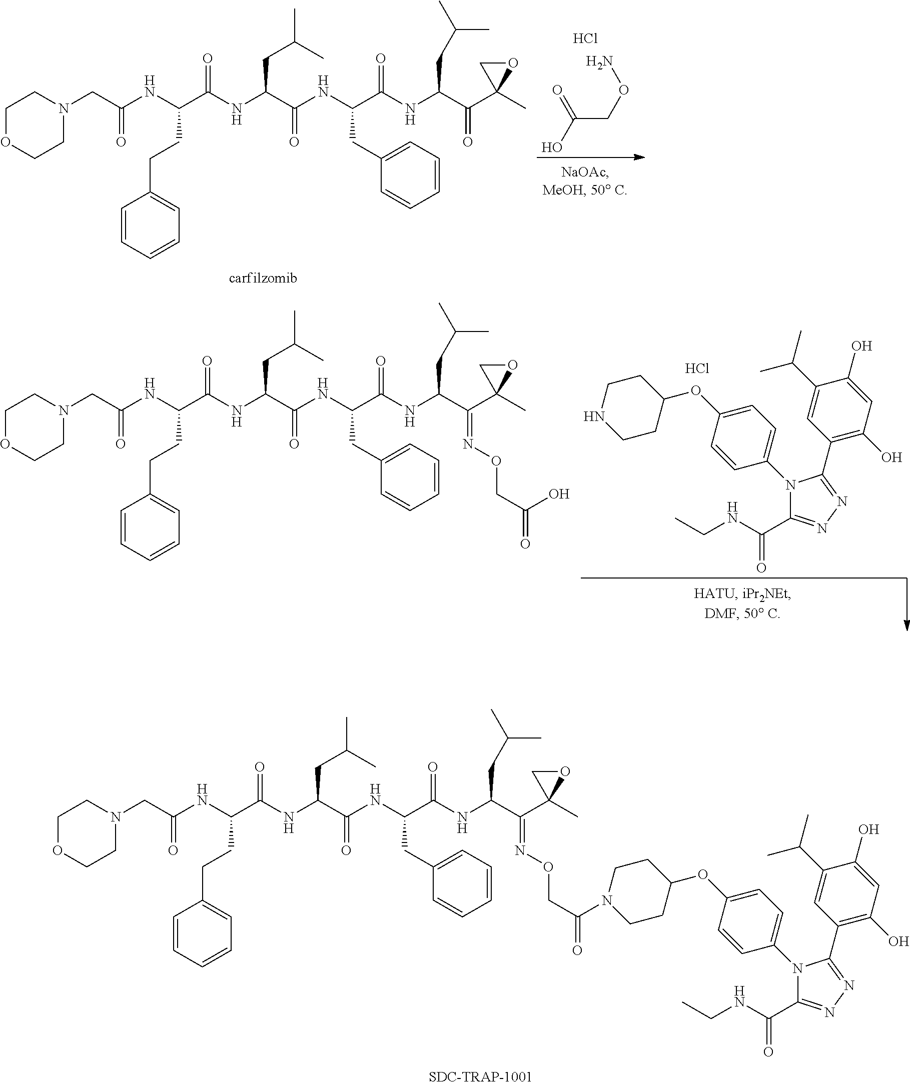

SDC-TRAP-1001: 4-(4-((1-((6S,9S,12S,15S,Z)-9-benzyl-6,12-diisobutyl-5-((S)-2-methyloxiran-2-yl)-18-morpholino-8,11,14,17-tetraoxo-15-phenethyl-3-oxa-4,7,10,13,16-pentaazaoctadec-4-en-1-oyl)piperidin-4-yl)oxy) phenyl)-5-(2,4-dihydroxy-5-isopropylphenyl)-N-ethyl-4H-1,2,4-triazole-3-carboxamide.

Carfilzomib (0.11 mmol) was dissolved in MeOH (1.5 mL), followed by the addition of O-(carboxymethyl)-hydroxylamine hemihydrochloride (0.22 mmol) and sodium acetate (0.44 mmol). The mixture was stirred in a 50° C. oil bath for 5 h, followed by the addition of formic acid (1 mL). The mixture was concentrated under reduced pressure, and the resulting residue was purified by silica gel chromatography (CH₂Cl₂/MeOH) to afford (6S,9S,12S,15S,Z)-9-benzyl-6,12-diisobutyl-5-((S)-2-methyloxiran-2-yl)-18-morpholino-8,11,14,17-tetraoxo-15-phenethyl-3-oxa-4,7,10,13,16-pentaazaoctadec-4-en-1-oic acid.

(6S,9S,12S,15S,Z)-9-benzyl-6,12-diisobutyl-5-((S)-2-methyloxiran-2-yl)-18-morpholino-8,11,14,17-tetraoxo-15-phenethyl-3-oxa-4,7,10,13,16-pentaazaoctadec-4-en-1-oic acid (0.08 mmol) was dissolved in DMF (1 mL), followed by the addition of 5-(2,4-dihydroxy-5-isopropylphenyl)-N-ethyl-4-(4-(piperidin-4-yloxy)phenyl)-4H-1,2,4-triazole-3-carboxamide hydrochloride (0.14 mmol), HATU (0.17 mmol) and diisopropyl ethylamine (0.33 mol). The solution was stirred at 50° C. for 2 h followed by the addition of 2M aqueous sodium hydroxide (1 mL). The solution was concentrated under reduced pressure, and the resulting residue was purified by silica gel chromatography (CH₂Cl₂/MeOH) to afford SDC-TRAP-1001 as a white solid.

¹H NMR (400 MHz, CD₃CN), δ 8.53 (t, J=8.0 Hz, 1H), 7.63-7.56 (m, 1H), 7.51 (br d, J=8.0 Hz, 1H), 7.23 (d, J=8.0 Hz, 2H), 7.20-7.17 (m, 2H), 7.11-7.02 (m, 11H), 6.76 (br d, J=6.76 Hz, 1H), 6.42 (s, 1H), 6.34 (br s, 1H), 4.78 (d, J=16.0 Hz, 1H), 4.74 (d, J=16.0 Hz, 1H), 4.61 (br s, 1H), 4.51 (ddd, J=8.0, 8.0, 8.0 Hz, 1H), 4.26-4.14 (m, 3H), 3.86-3.72 (m, 1H), 3.58-3.56 (m, 6H), 3.22-3.19 (m, 3H), 2.93-2.77 (m, 7H), 2.66 (d, J=4.0 Hz, 1H), 2.52-2.38 (m, 7H), 2.00-1.89 (m, 3H), 1.75-1.64 (m, 3H), 1.47-1.34 (m, 6H), 1.31 (s, 3H), 1.04 (t, J=8.0 Hz, 3H), 0.77 (d, J=8.0 Hz, 6H), 0.74 (d, J=8.0 Hz, 3H), 0.73 (d, J=8.0 Hz, 3H), 0.66 (d, J=8.0 Hz, 6H) ppm; ESMS calc'd for C₆₇H₈₉N₁₁O₁₂: 1239.7; found: 1240.4 (M+H⁺).

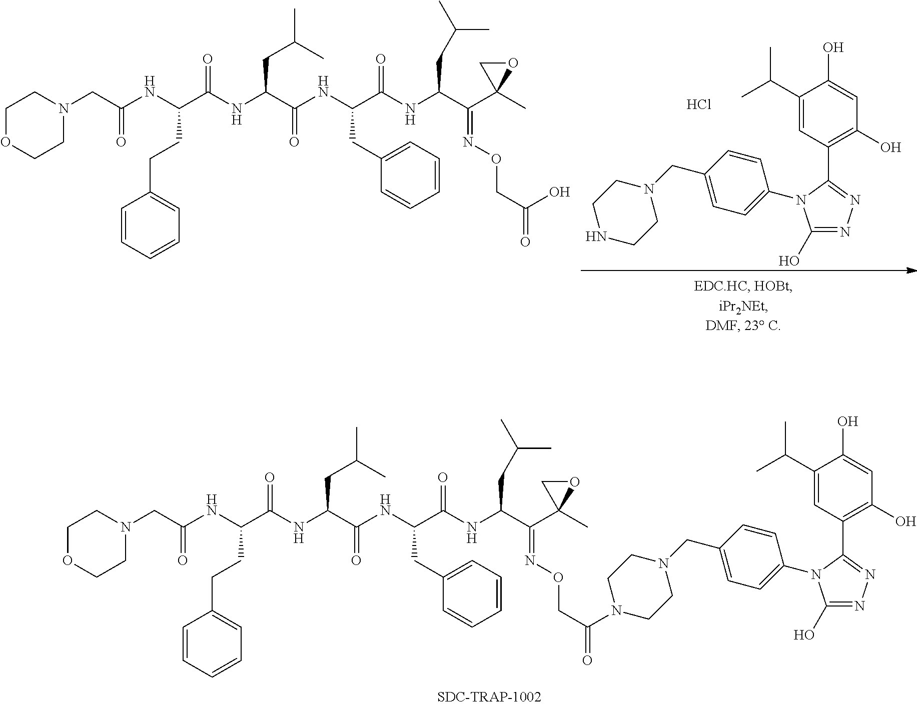

SDC-TRAP-1002: (S)—N—((S)-1-(((S,Z)-1-((2-(4-(4-(3-(2,4-dihydroxy-5-isopropylphenyl)-5-hydroxy-4H-1,2,4-triazol-4-yl)benzyl)piperazin-1-yl)-2-oxoethoxy)imino)-4-methyl-1-((S)-2-methyloxiran-2-yl)pentan-2-yl)amino)-1-oxo-3-phenylpropan-2-yl)-4-methyl-2-((S)-2-(2-morpholinoacetamido)-4-phenylbutanamido)pentanamide.

(6S,9S,12S,15S,Z)-9-benzyl-6,12-diisobutyl-5-((S)-2-methyloxiran-2-yl)-18-morpholino-8,11,14,17-tetraoxo-15-phenethyl-3-oxa-4,7,10,13,16-pentaazaoctadec-4-en-1-oic acid (0.10 mmol) was dissolved in DMF (2 mL), followed by the addition of 4-(5-hydroxy-4-(4-(piperazin-1-ylmethyl)phenyl)-4H-1,2,4-triazol-3-yl)-6-isopropylbenzene-1,3-diol hydrochloride (0.12 mmol), EDC.HCl (0.15 mmol), HOBt (0.12 mmol) and diisopropyl ethylamine (0.30 mmol). The solution was stirred at 23° C. for 17 h. The solution was concentrated under reduced pressure, and the resulting residue was purified by silica gel chromatography (CH₂Cl₂/MeOH) to afford SDC-TRAP-1002 as a white solid.

¹H NMR (400 MHz, CD₃CN), δ 9.89 (br s, 1H), 9.40 (br s, 1H, 8.45 (d, J=12.0 Hz, 1H), 7.98 (s, 1H), 7.50 (d, J=8.0 Hz, 1H), 7.39 (d, J=8.0 Hz, 2H), 7.20 (d, J=8.0 Hz, 2H), 7.18 (d, J=8.0 Hz, 2H), 7.11-7.03 (m, 8H), 6.74 (d, J=8.0 Hz, 1H), 6.47 (s, 1H), 6.28 (s, 1H), 4.73 (d, J=16.0 Hz, 1H), 4.68 (d, J=16.0 Hz, 1H), 4.48 (ddd, J=8.0, 8.0, 4.0 Hz, 1H), 4.24-4.12 (m, 3H), 3.59-3.56 (m, 4H), 3.52-3.48 (m, 3H), 3.33-3.28 (m, 2H), 3.19 (s, 1H), 2.94-2.75 (m, 6H), 2.66 (d, J=4.0 Hz, 1H), 2.53-2.48 (m, 2H), 2.42-2.35 (m, 7H), 2.03-1.95 (m, 4H), 1.73-1.67 (m, 2H), 1.45-1.32 (m, 4H), 1.30 (s, 3H), 0.76 (d, J=8.0 Hz, 6H), 0.74 (d, J=8.0 Hz, 3H), 0.73 (d, J=8.0 Hz, 3H), 0.65 (d, J=8.0 Hz, 6H); ESMS calc'd for C₆₄H₈₅N₁₁O₁₁: 1183.6; found: 1185.3 (M+R⁺).

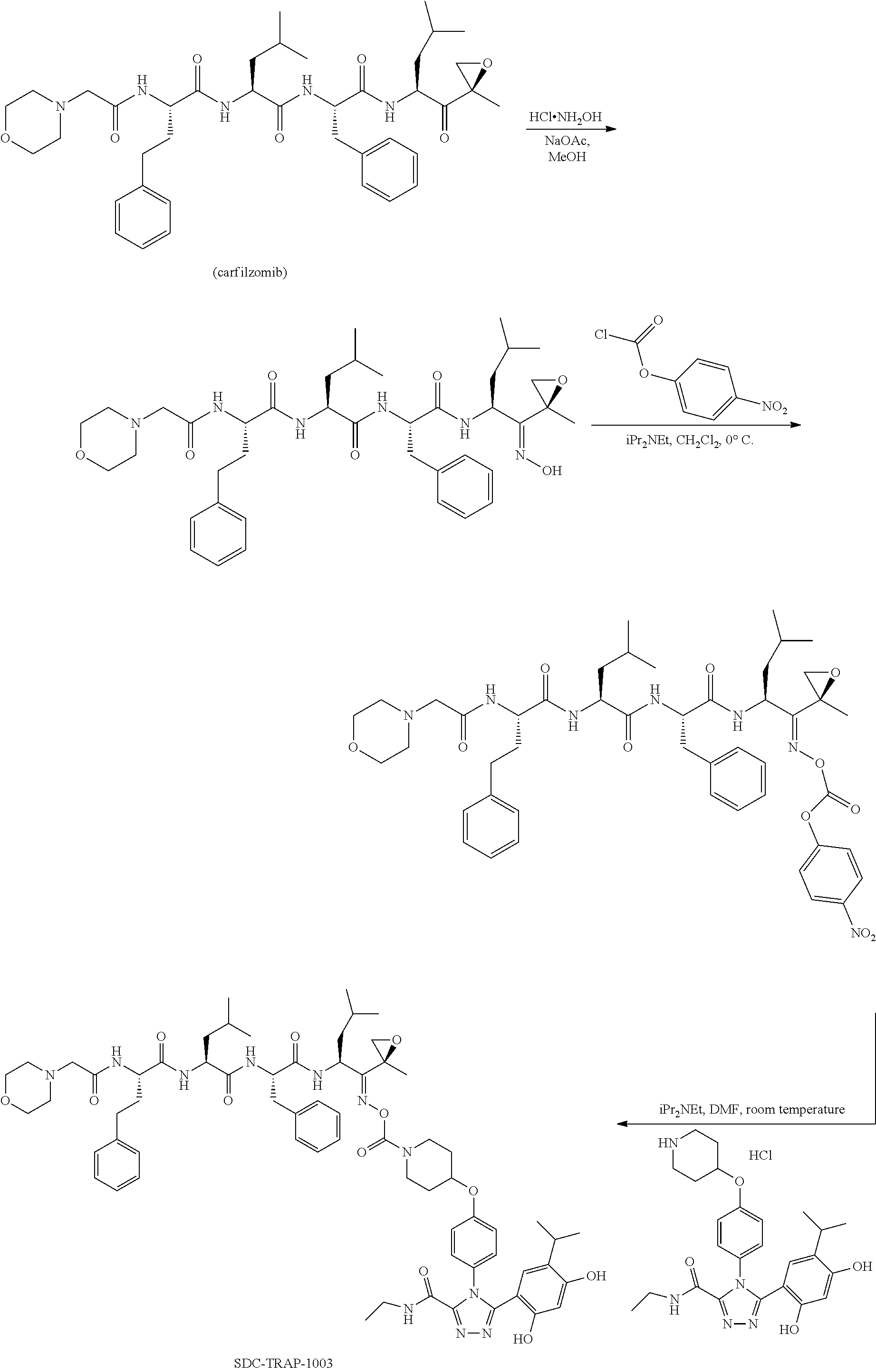

SDC-TRAP-1003: 4-(4-((1-((5 S,8S,11S,14S,Z)-8-benzyl-5,11-diisobutyl-4-((S)-2-methyloxiran-2-yl)-17-morpholino-7,10,13,16-tetraoxo-14-phenethyl-2-oxa-3,6,9,12,15-pentaazaheptadec-3-en-1-oyl)piperidin-4-yl)oxy)phenyl)-5-(2,4-dihydroxy-5-isopropylphenyl)-N-ethyl-4H-1,2,4-triazole-3-carboxamide.

Carfilzomib (0.15 mmol) was dissolved in MeOH (8 mL), followed by the addition of hydroxylamine hydrochloride (2.7 mmol) and sodium acetate (3.8 mmol). The mixture was stirred at 23° C. for 8 h. The mixture was concentrated under reduced pressure, and the resulting residue was purified by silica gel chromatography (CH₂Cl₂/MeOH) to afford (S)—N—((S)-1-(((S,Z)-1-(hydroxyimino)-4-methyl-1-((S)-2-methyloxiran-2-yl)pentan-2-yl)amino)-1-oxo-3-phenylpropan-2-yl)-4-methyl-2-((S)-2-(2-morpholinoacetamido)-4-phenylbutanamido)pentanamide.

(S)—N—((S)-1-(((S,Z)-1-(hydroxyimino)-4-methyl-1-((S)-2-methyloxiran-2-yl)pentan-2-yl)amino)-1-oxo-3-phenylpropan-2-yl)-4-methyl-2-((S)-2-(2-morpholinoacetamido)-4-phenylbutanamido)pentanamide (0.09 mmol) was dissolved in CH₂Cl₂ (2 mL), then cooled on an ice-water bath (0° C.). To the solution was added diisopropyl ethylamine (1.2 mmol) and 4-nitrophenylchloroformate (0.12 mmol). The solution was stirred for 15 h as the ice-water bath warmed up to 23° C. The solution was concentrated under reduced pressure, followed by the addition of 5-(2,4-dihydroxy-5-isopropylphenyl)-N-ethyl-4-(4-(piperidin-4-yloxy)phenyl)-4H-1,2,4-triazole-3-carboxamide hydrochloride (0.15 mmol), DMF (3 mL) and diisopropyl ethylamine (1.2 mmol). The solution was stirred at 23° C. for 1 h, then concentrated under reduced pressure. The resulting residue was purified by silica gel chromatography (CH₂Cl₂/MeOH) followed by reverse-phase C18 chromatography (H₂O/MeCN), followed by lyophilizing the desired fractions to yield SDC-TRAP-1003 as a white solid.

¹H NMR (400 MHz, CD₃OD), δ 8.16 (d, J=8.0 Hz, 1H), 8.09 (d, J=12.0 Hz, 1H), 8.06 (d, J=8.0 Hz, 1H), 7.19-7.15 (m, 4H), 7.12-6.93 (m, 12H), 6.55 (s, 1H), 6.24 (s, 1H), 4.71 (ddd, J=12.0, 12.0, 4.0 Hz, 1H), 4.62-4.56 (m, 2H), 4.33-4.29 (m, 3H), 3.70-3.65 (m, 8H), 3.47-3.43 (m, 2H), 3.09 (d, J=16.0 Hz, 1H), 3.05 (d, J=16.0 Hz, 1H), 3.02 (d, J=8.0 Hz, 2H), 2.91 (dddd, J=8.0, 8.0, 8.0, 8.0 Hz, 2H), 2.83 (d, J=4.0 Hz, 1H), 2.80-2.77 (m, 1H), 2.72 (d, J=4.0 Hz, 1H), 2.56-2.46 (m, 6H), 2.01-1.89 (m, 3H), 1.86-1.74 (m, 3H), 1.59-1.40 (m, 6H), 1.38 (s, 3H), 1.07 (t, J=8.0 Hz, 3H), 0.83 (d, J=8.0 Hz, 6H), 0.81 (d, J=8.0 Hz, 3H), 0.80 (d, J=8.0 Hz, 3H), 0.78 (d, J=8.0 Hz, 6H); ESMS calc'd for C₆₆H₈₇N₁₁O₁₂: 1225.7; found: 1226.8 (M+H⁺).

SDC-TRAP molecules which include a binding moiety which is an Hsp90 binding compound covalently linked to a non-reactive moiety on the peptide epoxy ketone molecule:

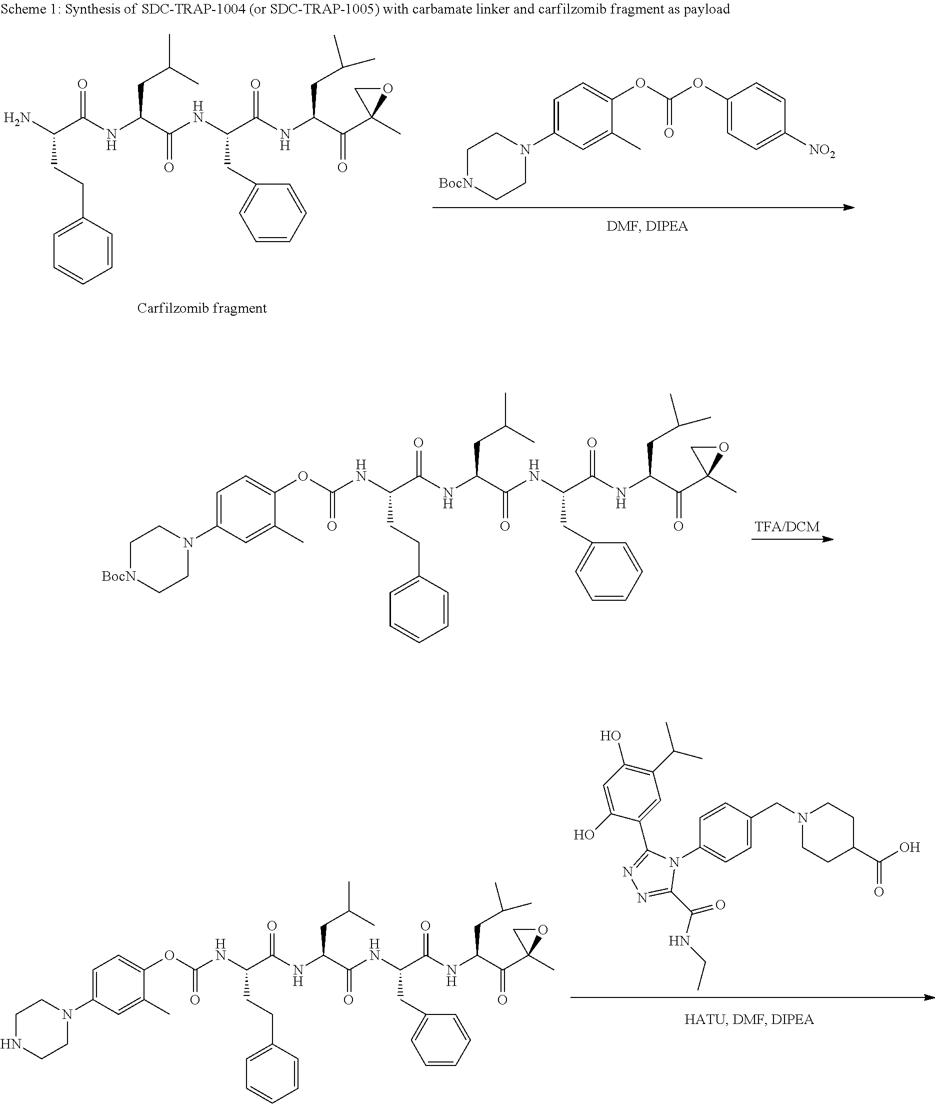

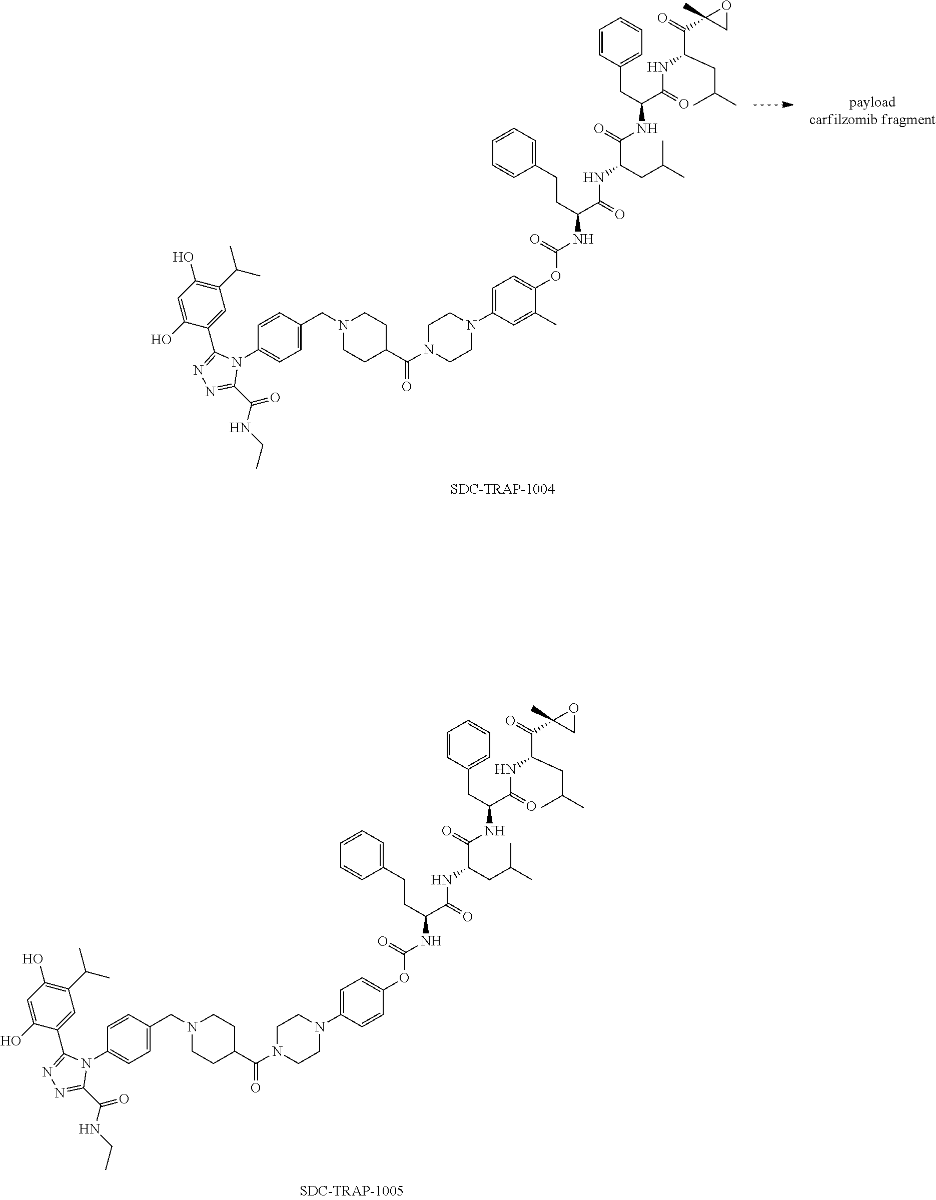 [SMILES: CCNC(=O)c1nnc(-c2cc(C(C)C)c(O)cc2O)n1-c1ccc(CN2CCC(C(=O)N3CCN(c4ccc(OC(=O)N[C@@H](CCc5ccccc5)C(=O)N[C@@H](CC(C)C)C(=O)N[C@@H](Cc5ccccc5)C(=O)N[C@@H](CC(C)C)C(=O)[C@@]5(C)CO5)c(C)c4)CC3)CC2)cc1 ; CCNC(=O)c1nnc(-c2cc(C(C)C)c(O)cc2O)n1-c1ccc(CN2CCC(C(=O)N3CCN(c4ccc(OC(=O)N[C@@H](CCc5ccccc5)C(=O)N[C@@H](CC(C)C)C(=O)N[C@@H](Cc5ccccc5)C(=O)N[C@@H](CC(C)C)C(=O)[C@@]5(C)CO5)cc4)CC3)CC2)cc1]

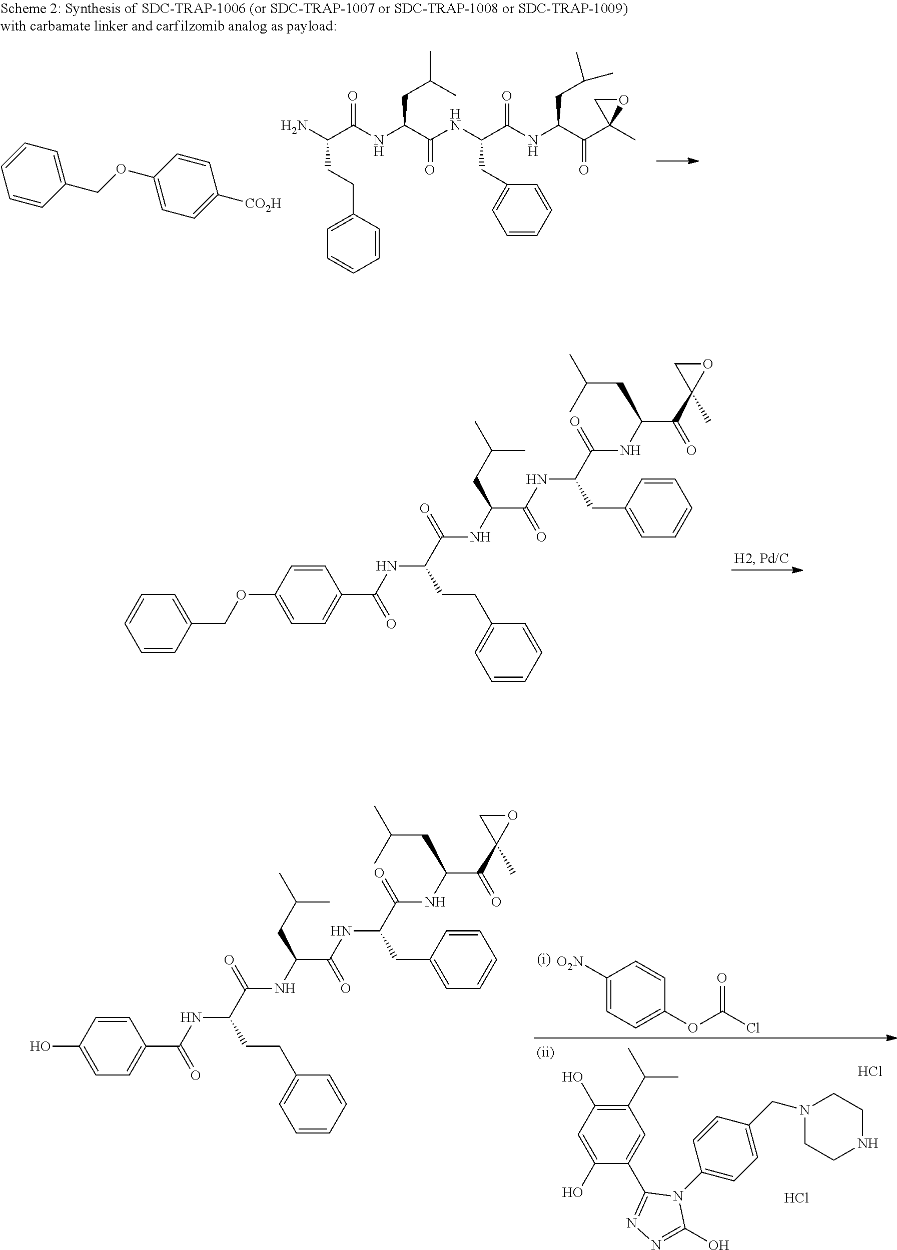

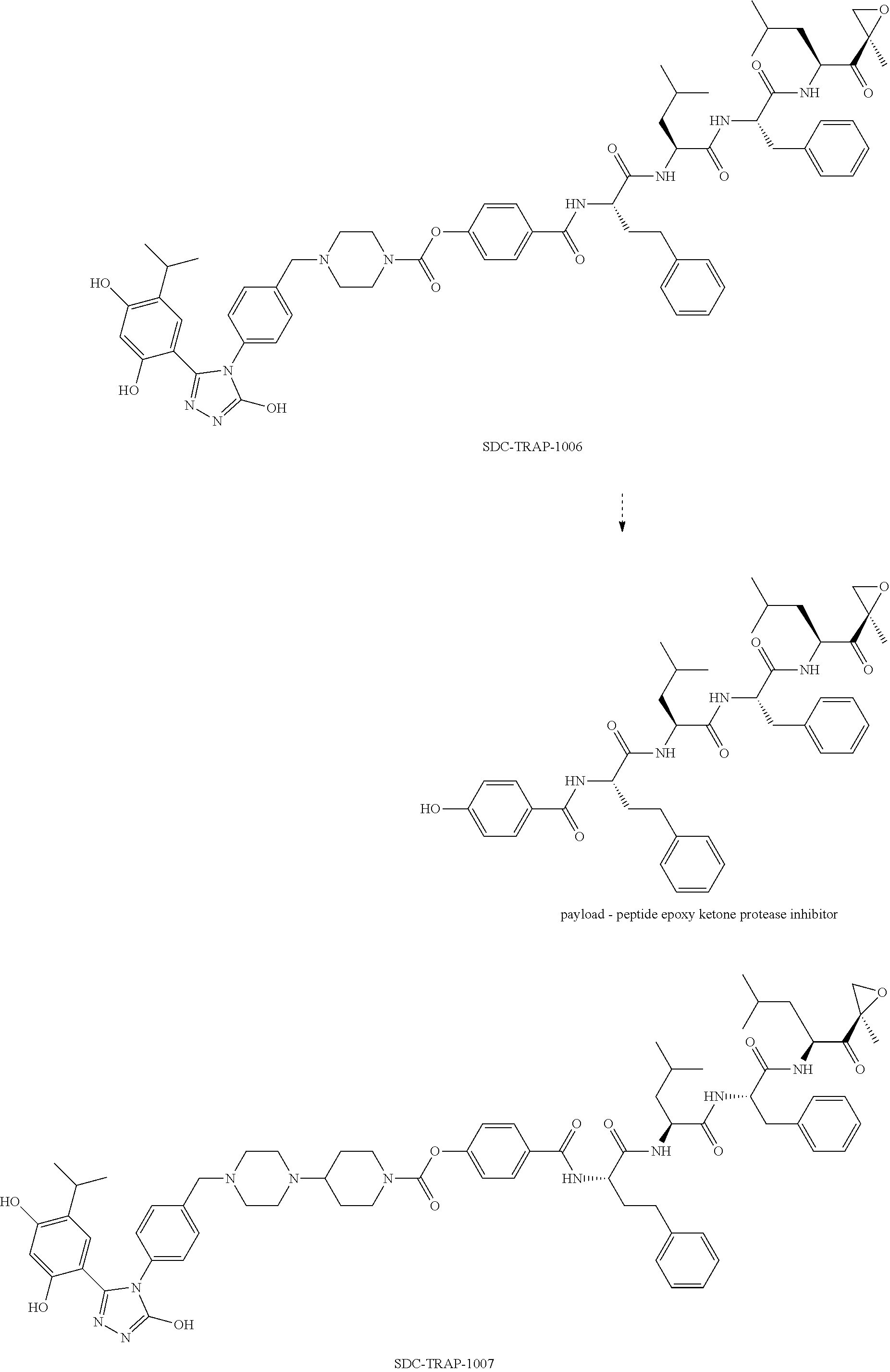 [SMILES: CC(C)C[C@H](NC(=O)[C@H](CCc1ccccc1)NC(=O)c1ccc(OC(=O)N2CCN(Cc3ccc(-n4c(O)nnc4-c4cc(C(C)C)c(O)cc4O)cc3)CC2)cc1)C(=O)N[C@@H](Cc1ccccc1)C(=O)N[C@@H](CC(C)C)C(=O)[C@@]1(C)CO1 ; CC(C)C[C@H](NC(=O)[C@H](CCc1ccccc1)NC(=O)c1ccc(O)cc1)C(=O)N[C@@H](Cc1ccccc1)C(=O)N[C@@H](CC(C)C)C(=O)[C@@]1(C)CO1 ; CC(C)C[C@H](NC(=O)[C@H](CCc1ccccc1)NC(=O)c1ccc(OC(=O)N2CCC(N3CCN(Cc4ccc(-n5c(O)nnc5-c5cc(C(C)C)c(O)cc5O)cc4)CC3)CC2)cc1)C(=O)N[C@@H](Cc1ccccc1)C(=O)N[C@@H](CC(C)C)C(=O)[C@@]1(C)CO1]

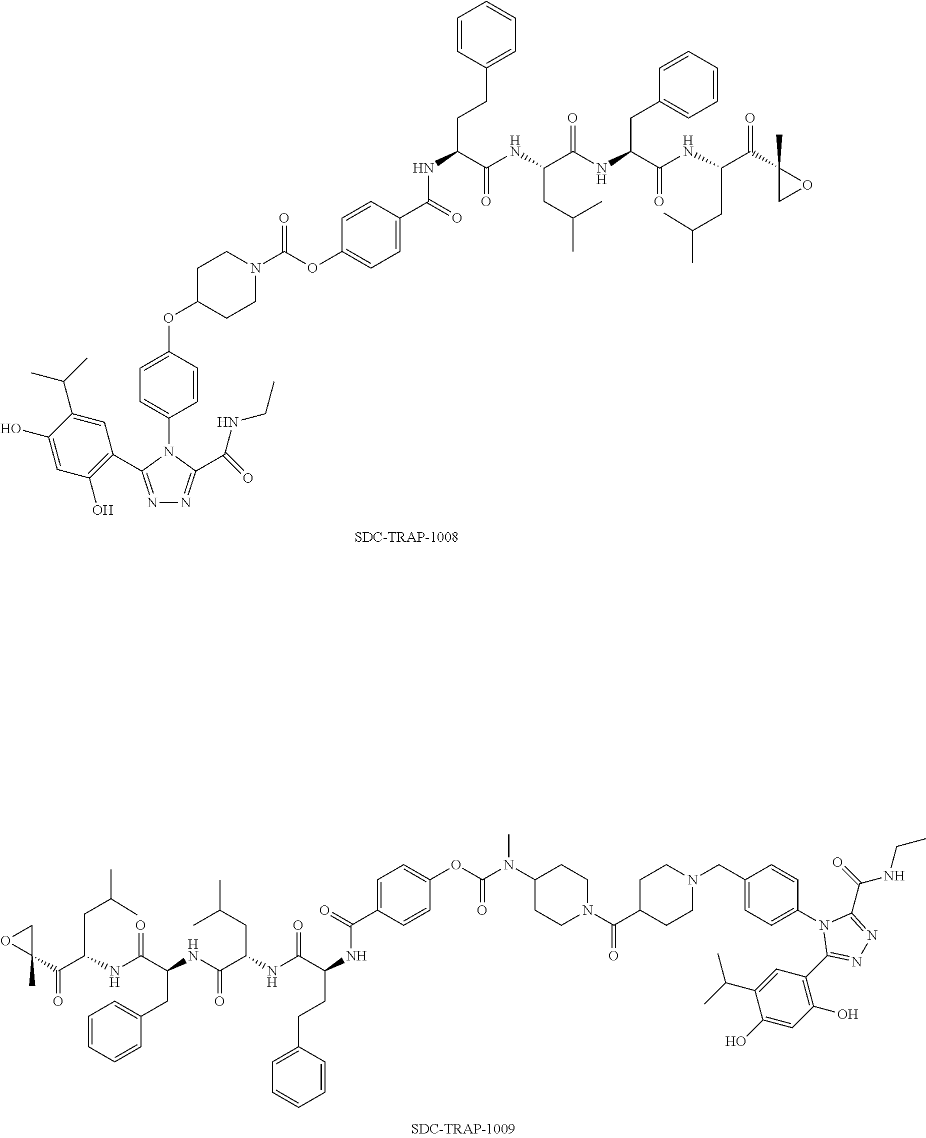 [SMILES: CCNC(=O)c1nnc(-c2cc(C(C)C)c(O)cc2O)n1-c1ccc(OC2CCN(C(=O)Oc3ccc(C(=O)N[C@@H](CCc4ccccc4)C(=O)N[C@@H](CC(C)C)C(=O)N[C@@H](Cc4ccccc4)C(=O)N[C@@H](CC(C)C)C(=O)[C@@]4(C)CO4)cc3)CC2)cc1 ; CCNC(=O)c1nnc(-c2cc(C(C)C)c(O)cc2O)n1-c1ccc(CN2CCC(C(=O)N3CCC(N(C)C(=O)Oc4ccc(C(=O)N[C@@H](CCc5ccccc5)C(=O)N[C@@H](CC(C)C)C(=O)N[C@@H](Cc5ccccc5)C(=O)N[C@@H](CC(C)C)C(=O)[C@@]5(C)CO5)cc4)CC3)CC2)cc1]

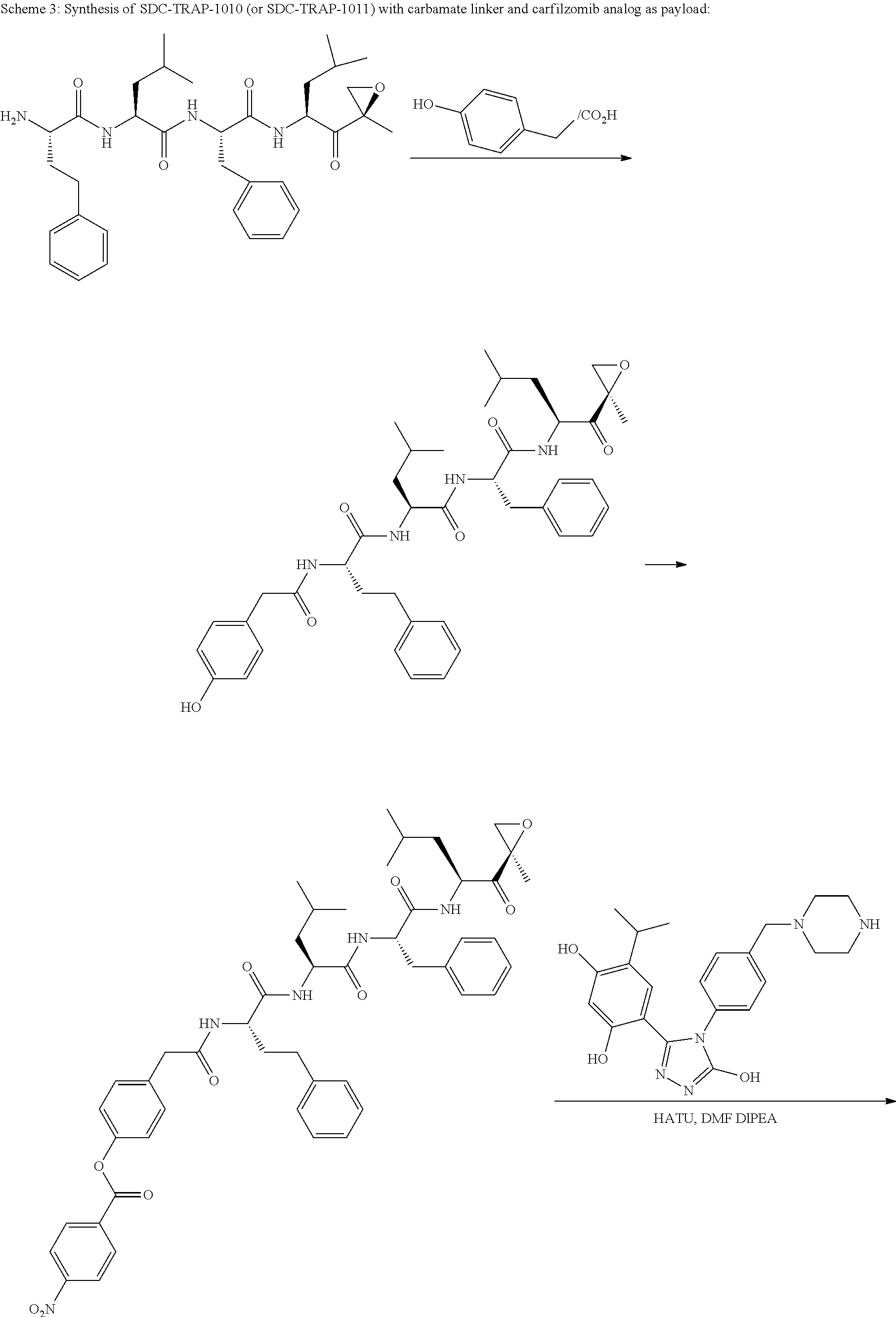

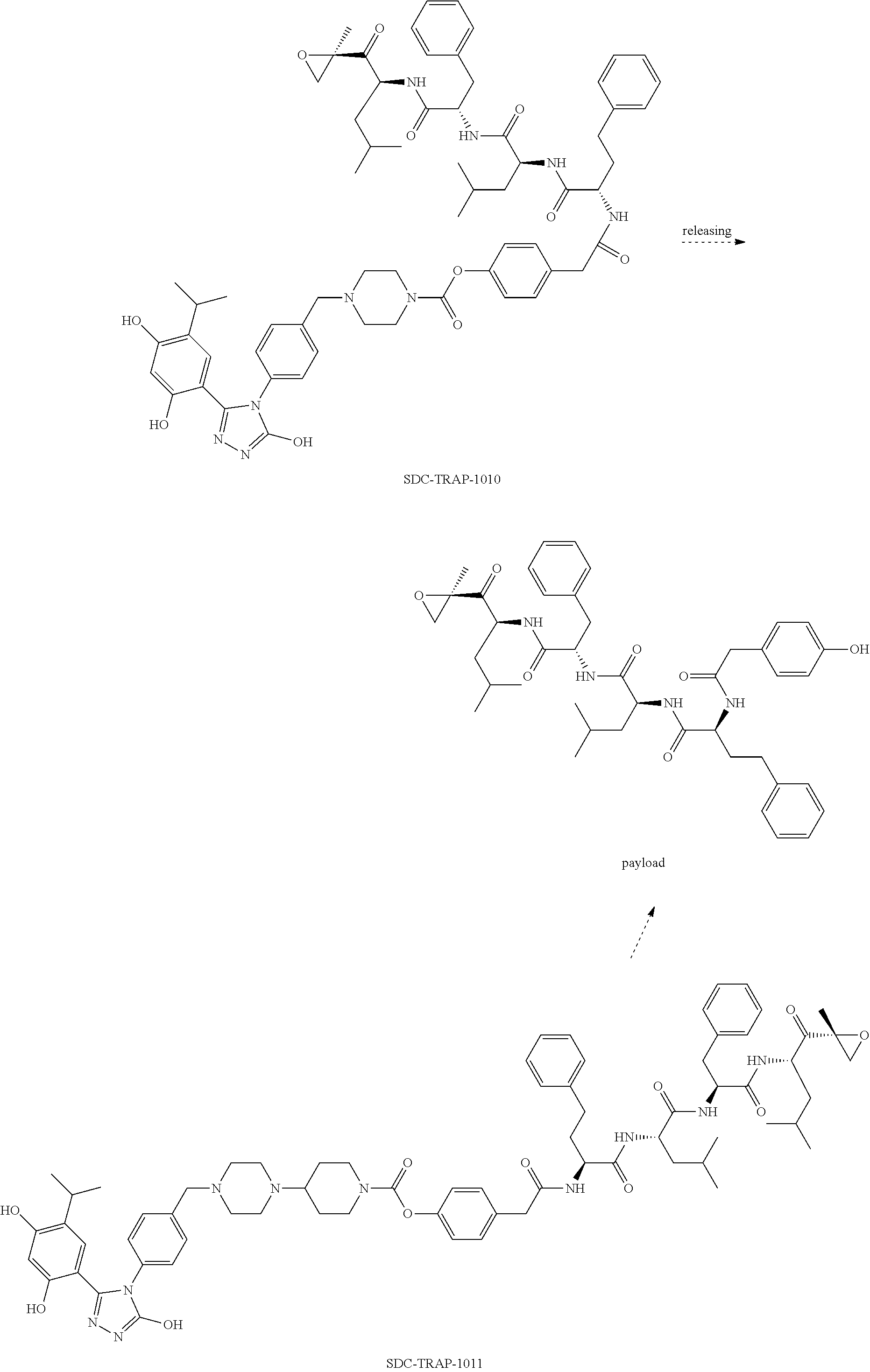 [SMILES: CC(C)C[C@H](NC(=O)[C@H](CCc1ccccc1)NC(=O)Cc1ccc(OC(=O)N2CCN(Cc3ccc(-n4c(O)nnc4-c4cc(C(C)C)c(O)cc4O)cc3)CC2)cc1)C(=O)N[C@@H](Cc1ccccc1)C(=O)N[C@@H](CC(C)C)C(=O)[C@@]1(C)CO1 ; CC(C)C[C@H](NC(=O)[C@H](CCc1ccccc1)NC(=O)Cc1ccc(O)cc1)C(=O)N[C@@H](Cc1ccccc1)C(=O)N[C@@H](CC(C)C)C(=O)[C@@]1(C)CO1 ; CC(C)C[C@H](NC(=O)[C@H](CCc1ccccc1)NC(=O)Cc1ccc(OC(=O)N2CCC(N3CCN(Cc4ccc(-n5c(O)nnc5-c5cc(C(C)C)c(O)cc5O)cc4)CC3)CC2)cc1)C(=O)N[C@@H](Cc1ccccc1)C(=O)N[C@@H](CC(C)C)C(=O)[C@@]1(C)CO1]

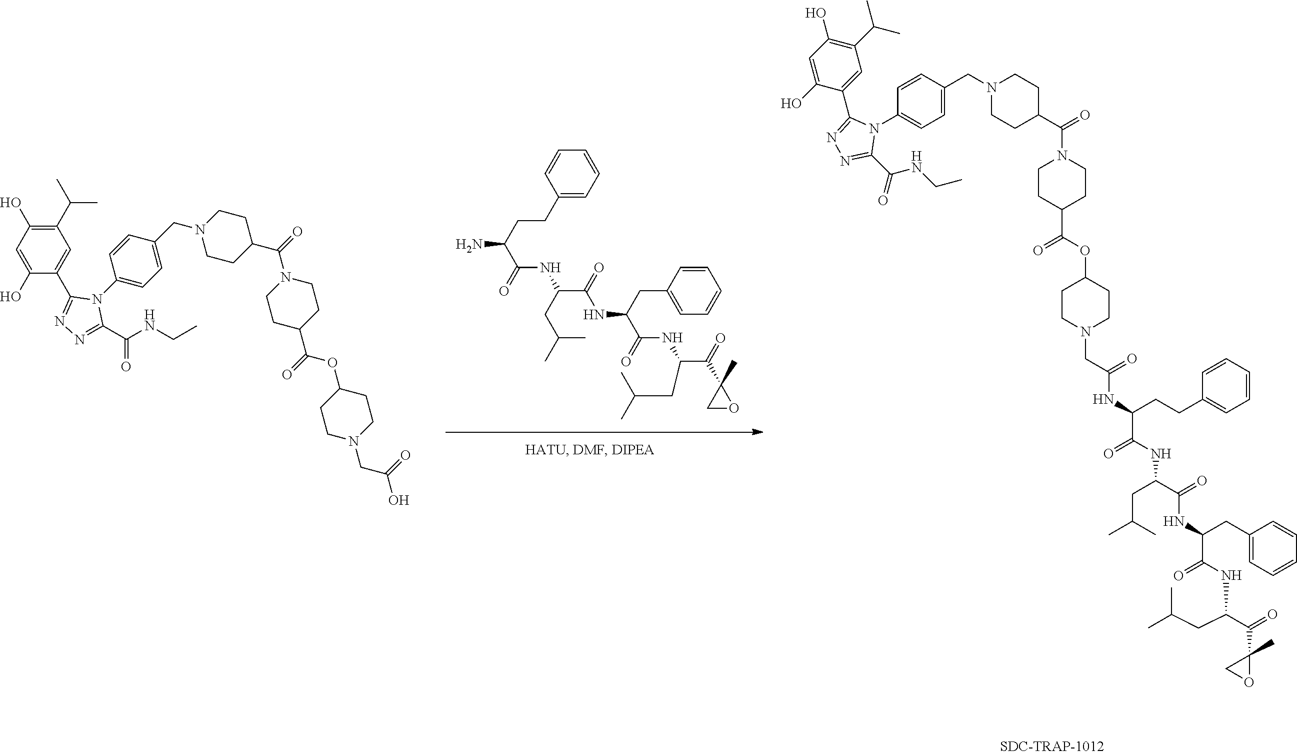

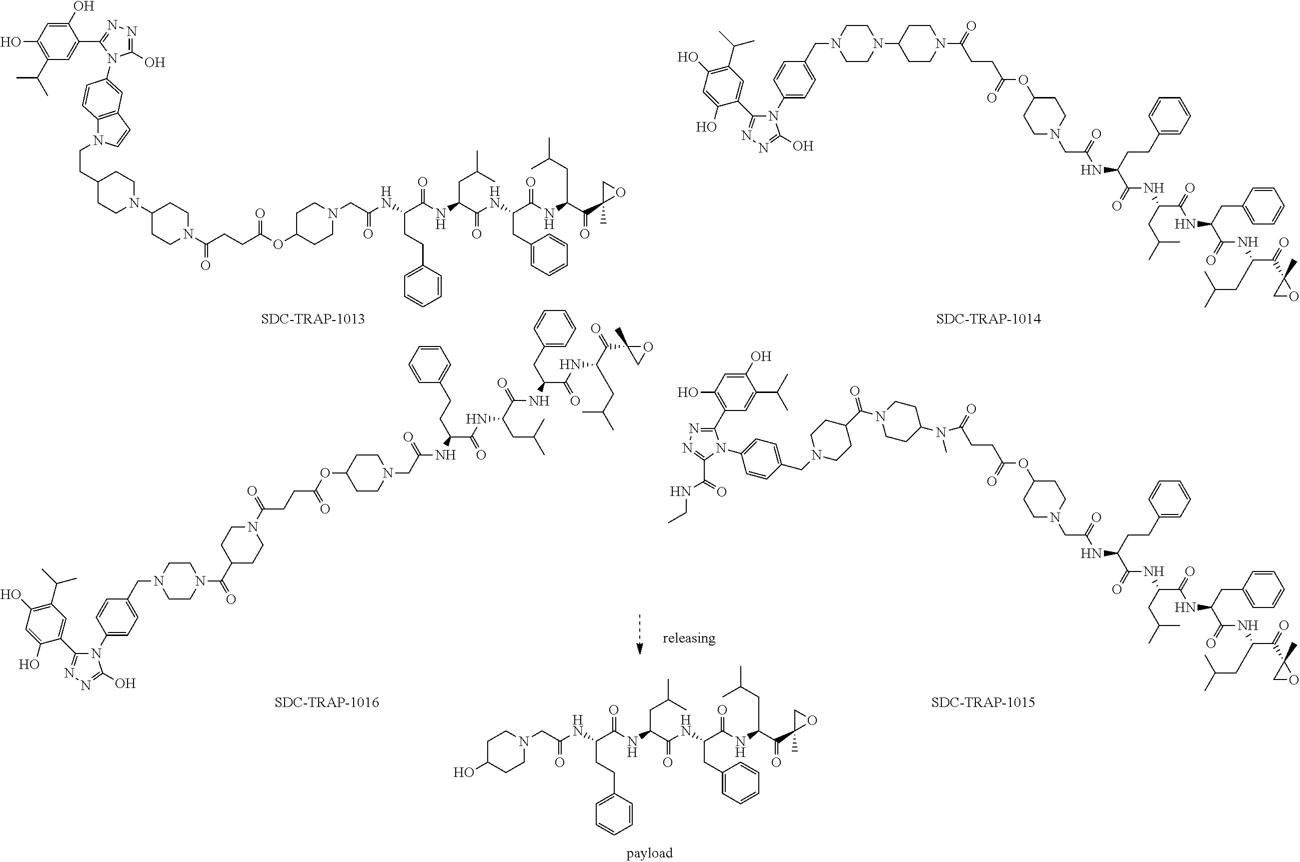

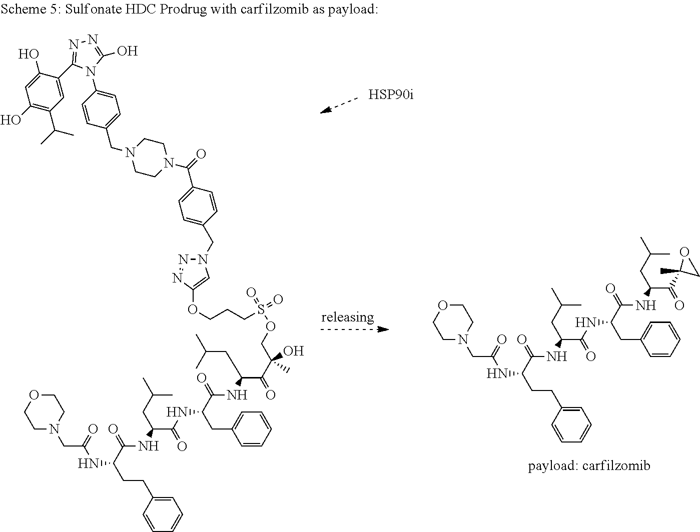 [SMILES: CC(C)C[C@H](NC(=O)[C@H](CCc1ccccc1)NC(=O)CN1CCOCC1)C(=O)N[C@@H](Cc1ccccc1)C(=O)N[C@@H](CC(C)C)C(=O)[C@](C)(O)COS(=O)(=O)CCCOc1cn(Cc2ccc(C(=O)N3CCN(Cc4ccc(-n5c(O)nnc5-c5cc(C(C)C)c(O)cc5O)cc4)CC3)cc2)nn1 ; CC(C)C[C@H](NC(=O)[C@H](CCc1ccccc1)NC(=O)CN1CCOCC1)C(=O)N[C@@H](Cc1ccccc1)C(=O)N[C@@H](CC(C)C)C(=O)[C@@]1(C)CO1]

### Example 1a

### 4-(4-(1-(4-(3-(2,4-dihydroxy-5-isopropylphenyl)-5-(ethylcarbamoyl)-4H-1,2,4-triazol-4-yl)benzyl)piperidine-4-carbonyl)piperazin-1-yl)-2-methylphenyl ((S)-1-(((S)-4-methyl-1-(((S)-1-(((S)-4-methyl-1-((R)-2-methyloxiran-2-yl)-1-oxopentan-2-yl)amino)-1-oxo-3-phenylpropan-2-yl)amino)-1-oxopentan-2-yl)amino)-1-oxo-4-phenylbutan-2-yl)carbamate (SDC-TRAP-1004)

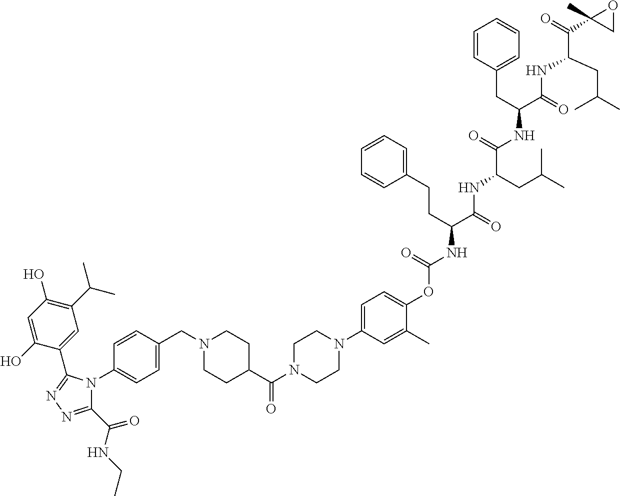 [SMILES: CCNC(=O)c1nnc(-c2cc(C(C)C)c(O)cc2O)n1-c1ccc(CN2CCC(C(=O)N3CCN(c4ccc(OC(=O)N[C@@H](CCc5ccccc5)C(=O)N[C@@H](CC(C)C)C(=O)N[C@@H](Cc5ccccc5)C(=O)N[C@@H](CC(C)C)C(=O)[C@@]5(C)CO5)c(C)c4)CC3)CC2)cc1]

### Step 1: Synthesis of tert-butyl 4-(3-methyl-4-((((S)-1-(((S)-4-methyl-1-(((S)-1-(((S)-4-methyl-1-((R)-2-methyloxiran-2-yl)-1-oxopentan-2-yl)amino)-1-oxo-3-phenylpropan-2-yl)amino)-1-oxopentan-2-yl)amino)-1-oxo-4-phenylbutan-2-yl)carbamoyl)oxy)phenyl)piperazine-1-carboxylate

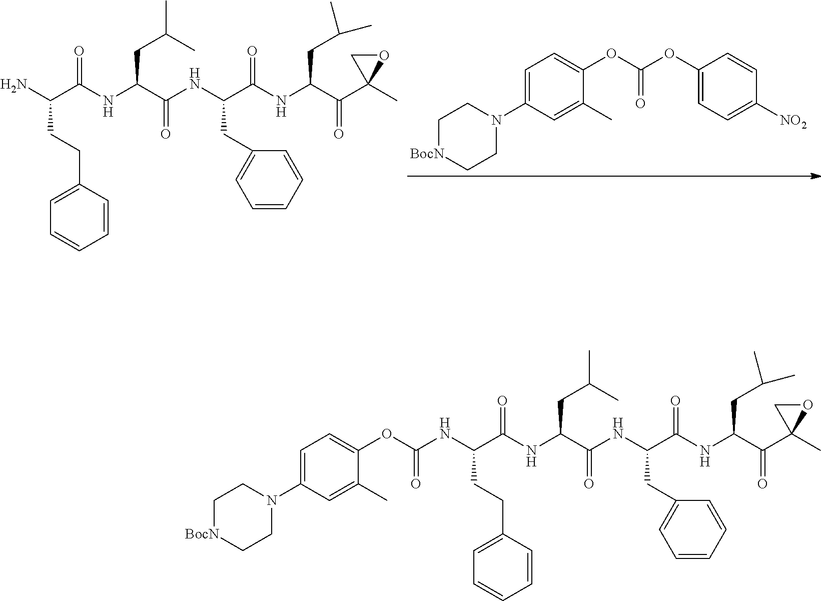

To a solution of (S)-2-((S)-2-amino-4-phenylbutanamido)-4-methyl-N—((S)-1-(((S)-4-methyl-1-((R)-2-methyloxiran-2-yl)-1-oxopentan-2-yl)amino)-1-oxo-3-phenylpropan-2-yl)pentanamide (24.4 mg, 0.053 mmol) and tert-butyl 4-(3-methyl-4-(((4-nitrophenoxy)carbonyl)oxy)phenyl)piperazine-1-carboxylate (60 mg) in DMF (1.0 mL) was added DIPEA (0.15 ml). The reaction mixture was stirred at room temperature overnight. The reaction mixture was diluted with water and extracted with ethyl acetate. After drying with Na₂SO₄, solvent was evaporated under reduced pressure to give a residue. The residue was purified by ISCO over silica gel to afford tert-butyl 4-(3-methyl-4-((((S)-1-(((S)-4-methyl-1-(((S)-1-(((S)-4-methyl-1-((R)-2-methyloxiran-2-yl)-1-oxopentan-2-yl)amino)-1-oxo-3-phenylpropan-2-yl)amino)-1-oxopentan-2-yl)amino)-1-oxo-4-phenylbutan-2-yl)carbamoyl)oxy)phenyl)piperazine-1-carboxylate (31 mg). ESMS calc'd for C₅₁H₇₀N₆O₉: 910.5; found: 911.2 (M+H)⁺.

### Step 2: Synthesis of 2-methyl-4-(piperazin-1-yl)phenyl ((S)-1-(((S)-4-methyl-1-(((S)-1-(((S)-4-methyl-1-((R)-2-methyloxiran-2-yl)-1-oxopentan-2-yl)amino)-1-oxo-3-phenylpropan-2-yl)amino)-1-oxopentan-2-yl)amino)-1-oxo-4-phenylbutan-2-yl)carbamate

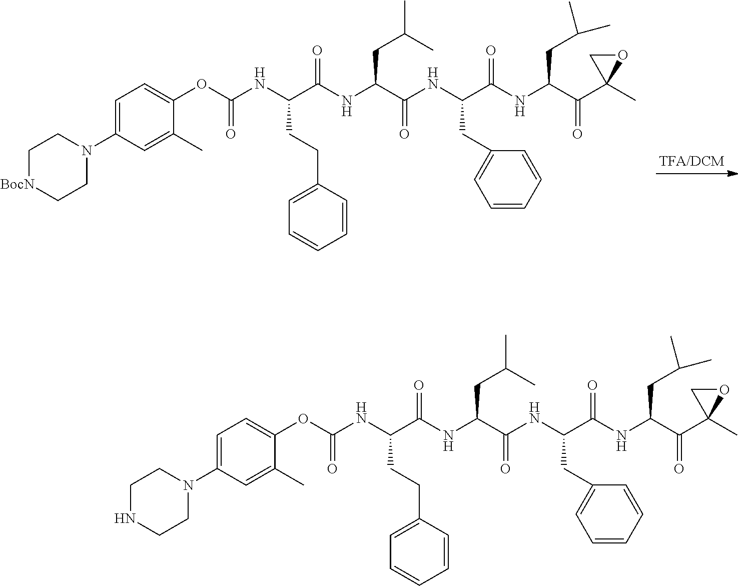 [SMILES: Cc1cc(N2CCN(C(=O)OC(C)(C)C)CC2)ccc1OC(=O)N[C@@H](CCc1ccccc1)C(=O)N[C@@H](CC(C)C)C(=O)N[C@@H](Cc1ccccc1)C(=O)N[C@@H](CC(C)C)C(=O)[C@@]1(C)CO1 ; Cc1cc(N2CCNCC2)ccc1OC(=O)N[C@@H](CCc1ccccc1)C(=O)N[C@@H](CC(C)C)C(=O)N[C@@H](Cc1ccccc1)C(=O)N[C@@H](CC(C)C)C(=O)[C@@]1(C)CO1]

To a solution of tert-butyl 4-(3-methyl-4-((((S)-1-(((S)-4-methyl-1-(((S)-1-(((S)-4-methyl-1-((R)-2-methyloxiran-2-yl)-1-oxopentan-2-yl)amino)-1-oxo-3-phenylpropan-2-yl)amino)-1-oxopentan-2-yl)amino)-1-oxo-4-phenylbutan-2-yl)carbamoyl)oxy)phenyl)piperazine-1-carboxylate (31 mg) in DCM (1.0 mL) was added TFA (0.20 mL). The reaction mixture was stirred at 0° C. for 30 minutes, and at room temperature for 1 hrs. Solvent was evaporated under a stream of nitrogen to give a residue, which was triturated with ether to afford 2-methyl-4-(piperazin-1-yl)phenyl ((S)-1-(((S)-4-methyl-1-(((S)-1-(((S)-4-methyl-1-((R)-2-methyloxiran-2-yl)-1-oxopentan-2-yl)amino)-1-oxo-3-phenylpropan-2-yl)amino)-1-oxopentan-2-yl)amino)-1-oxo-4-phenylbutan-2-yl)carbamate (30 mg) as a white solid. ESMS calc'd for C₄₆H₆₂N₆O₇: 810.5; found: 811.1 (M+H)⁺.

### Step 3: Synthesis of 4-(4-(1-(4-(3-(2,4-dihydroxy-5-isopropylphenyl)-5-(ethylcarbamoyl)-4H-1,2,4-triazol-4-yl)benzyl)piperidine-4-carbonyl)piperazin-1-yl)-2-methylphenyl ((S)-1-(((S)-4-methyl-1-(((S)-1-(((S)-4-methyl-1-((R)-2-methyloxiran-2-yl)-1-oxopentan-2-yl)amino)-1-oxo-3-phenylpropan-2-yl)amino)-1-oxopentan-2-yl)amino)-1-oxo-4-phenylbutan-2-yl)carbamate

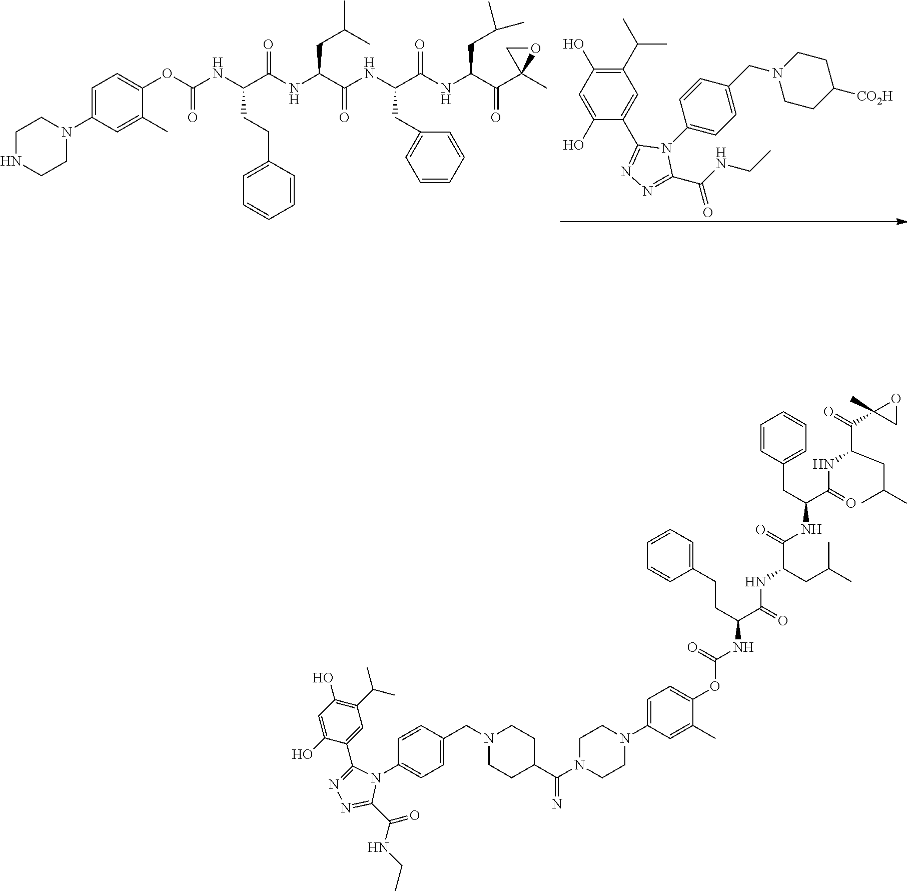

To a solution of 2-methyl-4-(piperazin-1-yl)phenyl ((S)-1-(((S)-4-methyl-1-(((S)-1-(((S)-4-methyl-1-((R)-2-methyloxiran-2-yl)-1-oxopentan-2-yl)amino)-1-oxo-3-phenylpropan-2-yl)amino)-1-oxopentan-2-yl)amino)-1-oxo-4-phenylbutan-2-yl)carbamate (30 mg) and 1-(4-(3-(2,4-dihydroxy-5-isopropylphenyl)-5-(ethylcarbamoyl)-4H-1,2,4-triazol-4-yl)benzyl)piperidine-4-carboxylic acid (41 mg, 0.08 mmol) in DMF (2.0 mL) was added HATU (30 mg, 0.08 mmol) and DIPEA (0.10 mL). The mixture was stirred at room temperature overnight. Solvent was evaporated under reduced pressure to give a residue, which was purified by ISCO over silica gel to afford the title compound (SDC-TRAP-1004) (7.1 mg) as a white solid. ESMS calc'd for C₇₃H₉₃N₁₁O₁₁: 1299.7; found: 1300.5 (M+H)⁺.

### Example 1b

### 4-(4-(1-(4-(3-(2,4-dihydroxy-5-isopropylphenyl)-5-(ethylcarbamoyl)-4H-1,2,4-triazol-4-yl)benzyl)piperidine-4-carbonyl)piperazin-1-yl)phenyl ((S)-1-(((S)-4-methyl-1-(((S)-1-(((S)-4-methyl-1-((R)-2-methyloxiran-2-yl)-1-oxopentan-2-yl)amino)-1-oxo-3-phenylpropan-2-yl)amino)-1-oxopentan-2-yl)amino)-1-oxo-4-phenylbutan-2-yl)carbamate (SDC-TRAP-1005)

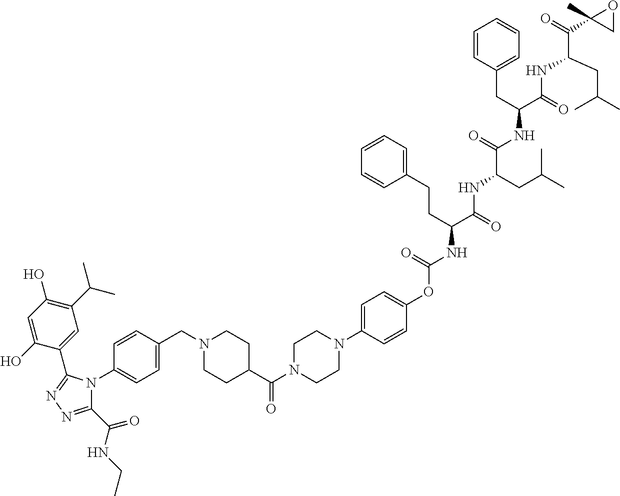 [SMILES: CCNC(=O)c1nnc(-c2cc(C(C)C)c(O)cc2O)n1-c1ccc(CN2CCC(C(=O)N3CCN(c4ccc(OC(=O)N[C@@H](CCc5ccccc5)C(=O)N[C@@H](CC(C)C)C(=O)N[C@@H](Cc5ccccc5)C(=O)N[C@@H](CC(C)C)C(=O)[C@@]5(C)CO5)cc4)CC3)CC2)cc1]

The title compound was prepared analogously using a similar procedure to that for SDC-TRAP-1004. ¹H NMR (400 MHz, DMSO-d₆) 10.62 (s, 1H), 9.76 (s, 1H), 8.96 (t, J=5.9 Hz, 1H), 8.23 (d, J=7.6 Hz, 1H), 7.98 (d, J=8.0 Hz, 1H), 7.93 (d, J=8.0 Hz, 2H), 7.38 (d, J=8.3 Hz, 2H), 7.33-7.25 (m, 5H), 7.23-6.90 (m, 11H), 6.58 (s, 1H), 6.34 (s, 1H), 4.56-4.50 (m, 1H), 4.39-4.28 (m, 2H), 4.08-3.95 (m, 1H), 3.61 (d, J=17.2 Hz, 4H), 3.49 (s, 2H), 3.22-0.75 (m, 54H). ESMS calc'd for C₇₂H₉₁N₁₁O₁₁: 1285.6; found: 1286.3 (M+H)⁺.

### Example 1c

### (4-(((S)-1-(((S)-4-methyl-1-(((S)-1-(((S)-4-methyl-1-((R)-2-methyloxiran-2-yl)-1-oxopentan-2-yl)amino)-1-oxo-3-phenylpropan-2-yl)amino)-1-oxopentan-2-yl)amino)-1-oxo-4-phenylbutan-2-yl)carbamoyl)phenyl 4-(4-(3-(2,4-dihydroxy-5-isopropylphenyl)-5-hydroxy-4H-1,2,4-triazol-4-yl)benzyl)piperazine-1-carboxylate (SDC-TRAP-1006)

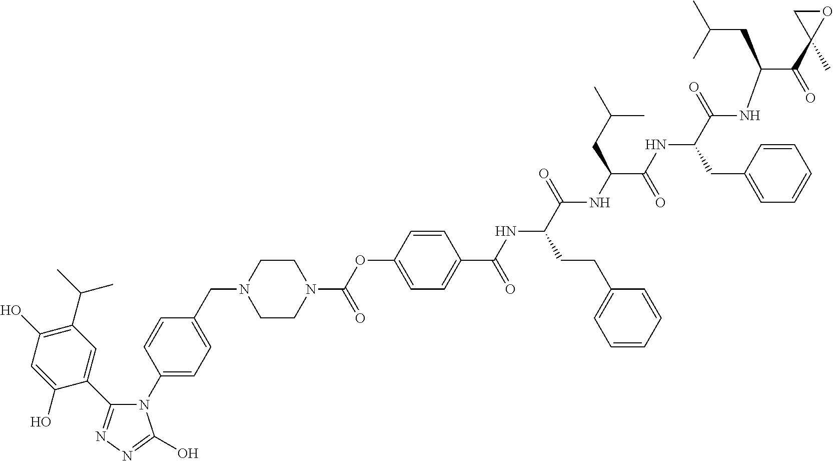 [SMILES: CC(C)C[C@H](NC(=O)[C@H](CCc1ccccc1)NC(=O)c1ccc(OC(=O)N2CCN(Cc3ccc(-n4c(O)nnc4-c4cc(C(C)C)c(O)cc4O)cc3)CC2)cc1)C(=O)N[C@@H](Cc1ccccc1)C(=O)N[C@@H](CC(C)C)C(=O)[C@@]1(C)CO1]

### Step 1: Synthesis of 4-((benzyloxy)carbonyl)phenyl 4-(4-(3-(2,4-dihydroxy-5-isopropylphenyl)-5-hydroxy-4H-1,2,4-triazol-4-yl)benzyl)piperazine-1-carboxylate

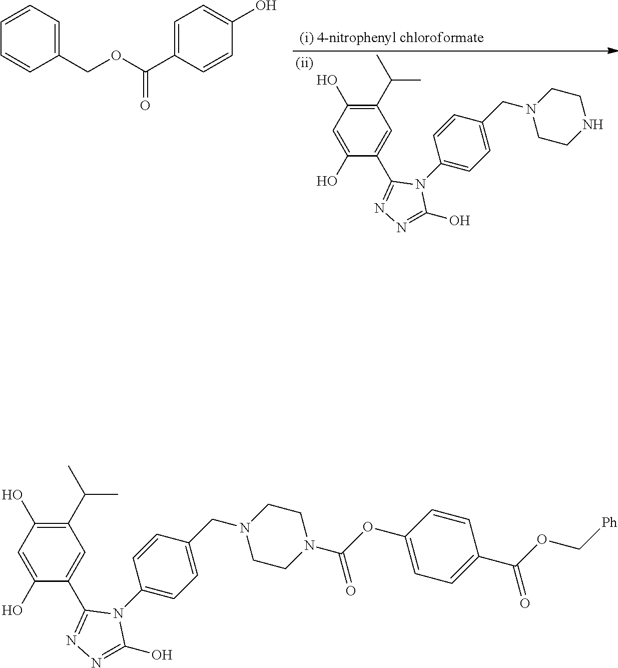 [SMILES: O=C(OCc1ccccc1)c1ccc(O)cc1 ; CC(C)c1cc(-c2nnc(O)n2-c2ccc(CN3CCNCC3)cc2)c(O)cc1O ; CC(C)c1cc(-c2nnc(O)n2-c2ccc(CN3CCN(C(=O)Oc4ccc(C(=O)OCc5ccccc5)cc4)CC3)cc2)c(O)cc1O]

To a solution of benzyl 4-hydroxybenzoate (137 mg, 0.60 mmol) and 4-nitrophenyl chloroformate (120 mg, 0.60 mmol) in THF (5.0 mL) was added DIPEA (0.20 ml). The reaction mixture was stirred at room temperature to 2 hrs. LC-MS showed the desired product of benzyl 4-(((4-nitrophenoxy)carbonyl)oxy)benzoate. The above reaction mixture and DIPEA (0.20 mL) was added to a solution of 4-(5-hydroxy-4-(4-(piperazin-1-ylmethyl)phenyl)-4H-1,2,4-triazol-3-yl)-6-isopropylbenzene-1,3-diol hydrochloride salt (289 mg, 0.60 mmol) in DMF (5.0 mL). The reaction mixture was stirred at room temperature for 1 hr. Solvents were evaporated under reduced pressure to give a residue, which was purified by ISCO over silica gel to afford 4-((benzyloxy)carbonyl)phenyl 4-(4-(3-(2,4-dihydroxy-5-isopropylphenyl)-5-hydroxy-4H-1,2,4-triazol-4-yl)benzyl)piperazine-1-carboxylate (240 mg, 60%). ESMS calc'd for C₃₇H₃₇N₅O₇: 663.3; found: 664.0 (M+H)⁺.

### Step 2: 4-((4-(4-(3-(2,4-dihydroxy-5-isopropylphenyl)-5-hydroxy-4H-1,2,4-triazol-4-yl)benzyl)piperazine-1-carbonyl)oxy)benzoic acid

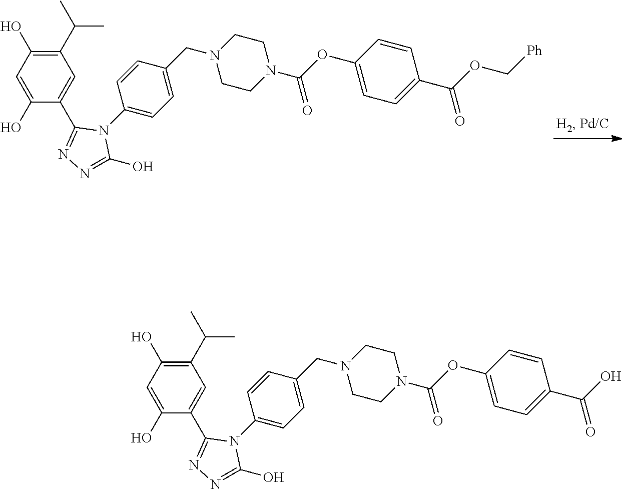 [SMILES: CC(C)c1cc(-c2nnc(O)n2-c2ccc(CN3CCN(C(=O)Oc4ccc(C(=O)OCc5ccccc5)cc4)CC3)cc2)c(O)cc1O ; CC(C)c1cc(-c2nnc(O)n2-c2ccc(CN3CCN(C(=O)Oc4ccc(C(=O)O)cc4)CC3)cc2)c(O)cc1O]

To a solution of 4-((benzyloxy)carbonyl)phenyl 4-(4-(3-(2,4-dihydroxy-5-isopropylphenyl)-5-hydroxy-4H-1,2,4-triazol-4-yl)benzyl)piperazine-1-carboxylate (240 mg, 0.36 mmol) in DCM (30 mL) and MeOH (5. mL) was added 10% Pd/C (100 mg). The reaction mixture was stirred at room temperature for 4 hrs under a hydrogen balloon. The reaction mixture was filtered through celite. Solvents were evaporated under reduced pressure to give a residue, which was purified by ISCO over silica gel to afford 4-((4-(4-(3-(2,4-dihydroxy-5-isopropylphenyl)-5-hydroxy-4H-1,2,4-triazol-4-yl)benzyl)piperazine-1-carbonyl)oxy)benzoic acid (134 mg, 65%). ESMS calc'd for C₃₀H₃₁N₅O₇: 573.2; found: 574.0 (M+H)⁺.

### Step 3: Synthesis of (4-(((S)-1-(((S)-4-methyl-1-(((S)-1-(((S)-4-methyl-1-((R)-2-methyloxiran-2-yl)-1-oxopentan-2-yl)amino)-1-oxo-3-phenylpropan-2-yl)amino)-1-oxopentan-2-yl)amino)-1-oxo-4-phenylbutan-2-yl)carbamoyl)phenyl 4-(4-(3-(2,4-dihydroxy-5-isopropylphenyl)-5-hydroxy-4H-1,2,4-triazol-4-yl)benzyl)piperazine-1-carboxylate

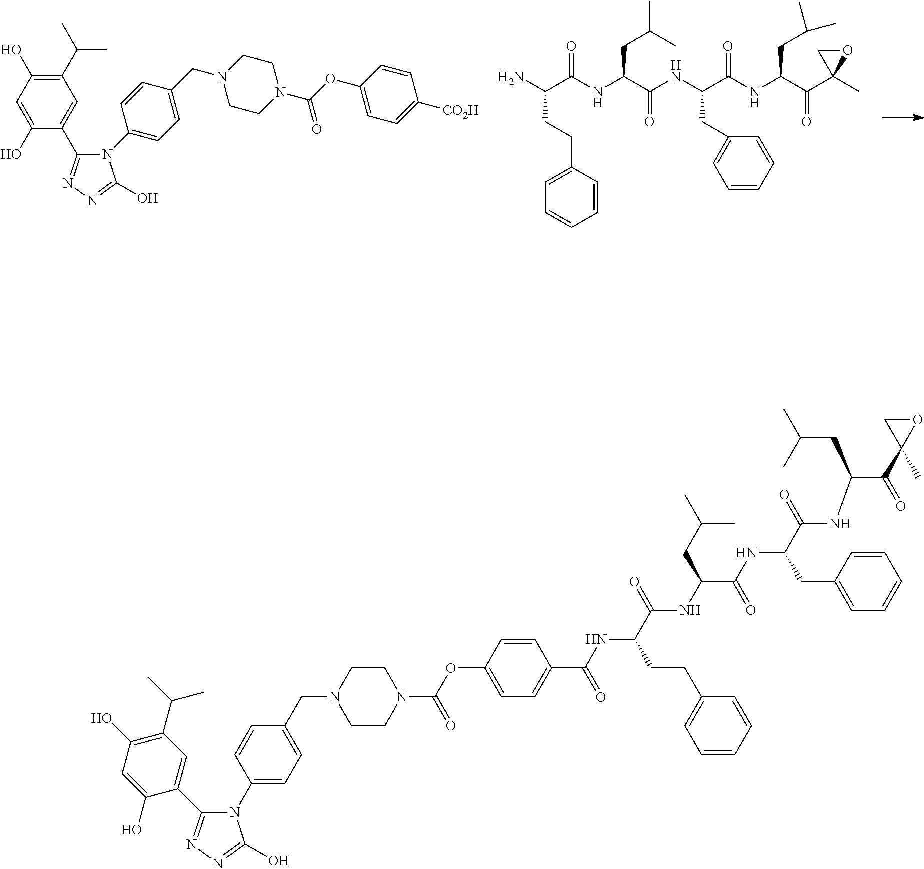 [SMILES: CC(C)c1cc(-c2nnc(O)n2-c2ccc(CN3CCN(C(=O)Oc4ccc(C(=O)O)cc4)CC3)cc2)c(O)cc1O ; CC(C)C[C@H](NC(=O)[C@@H](N)CCc1ccccc1)C(=O)N[C@@H](Cc1ccccc1)C(=O)N[C@@H](CC(C)C)C(=O)[C@@]1(C)CO1 ; CC(C)C[C@H](NC(=O)[C@H](CCc1ccccc1)NC(=O)c1ccc(OC(=O)N2CCN(Cc3ccc(-n4c(O)nnc4-c4cc(C(C)C)c(O)cc4O)cc3)CC2)cc1)C(=O)N[C@@H](Cc1ccccc1)C(=O)N[C@@H](CC(C)C)C(=O)[C@@]1(C)CO1]

To a solution of 4-((4-(4-(3-(2,4-dihydroxy-5-isopropylphenyl)-5-hydroxy-4H-1,2,4-triazol-4-yl)benzyl)piperazine-1-carbonyl)oxy)benzoic acid (63 mg, 0.11 mmol) and (S)-2-((S)-2-amino-4-phenylbutanamido)-4-methyl-N—((S)-1-(((S)-4-methyl-1-((R)-2-methyloxiran-2-yl)-1-oxopentan-2-yl)amino)-1-oxo-3-phenylpropan-2-yl)pentanamide (78 mg, 0.11 mmol) in DMF (4.0 mL) was added HATU (42 mg, 0.11 mmol) and DIPEA (0.120 mL). The mixture was stirred at room temperature for 2 hrs. Solvent was evaporated under reduced pressure to give a residue, which was purified by ISCO over silica gel to afford the title compound (SDC-TRAP-1006) (30 mg) as a white solid. ¹H NMR (400 MHz, DMSO-d₆) δ 11.92 (s, 1H), 9.59 (s, 1H), 9.40 (s, 1H), 8.55 (d, J=7.7 Hz, 1H), 8.20 (d, J=7.4 Hz, 1H), 7.97 (d, J=8.0 Hz, 1H), 7.92-7.85 (m, 3H), 7.35 (d, J=8.0 Hz, 2H), 7.30-7.05 (m, 14H), 6.78 (s, 1H), 6.26 (s, 1H), 4.56-4.23 (m, 4H), 3.60 (brs, 2H), 3.52 (s, 2H), 3.44 (brs, 2H), 3.11 (d, J=5.2 Hz, 1H), 3.01-2.94 (m, 3H), 2.77-0.77 (m, 36H). ESMS calc'd for C₆₄H₇₇N₉O₁₁: 1147.6; found: 1148.1 (M+H)⁺.

### Example 1 d

### 4-(((S)-1-(((S)-4-methyl-1-(((S)-1-(((S)-4-methyl-1-((R)-2-methyloxiran-2-yl)-1-oxopentan-2-yl)amino)-1-oxo-3-phenylpropan-2-yl)amino)-1-oxopentan-2-yl)amino)-1-oxo-4-phenylbutan-2-yl)carbamoyl)phenyl 4-(4-(4-(3-(2,4-dihydroxy-5-isopropylphenyl)-5-hydroxy-4H-1,2,4-triazol-4-yl)benzyl)piperazin-1-yl)piperidine-1-carboxylate (SDC-TRAP-1007)

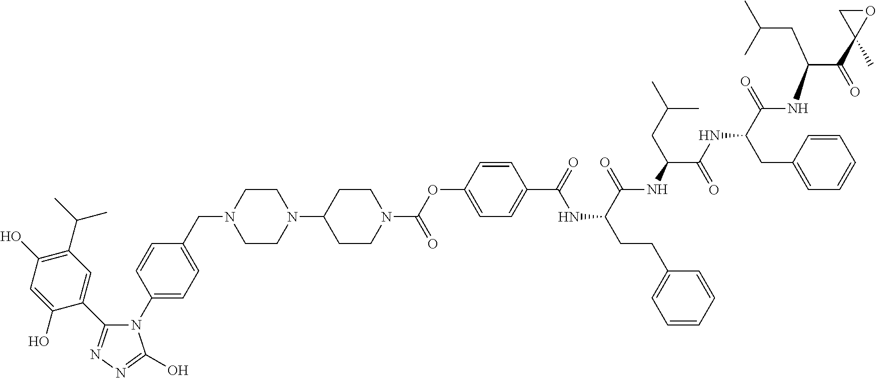 [SMILES: CC(C)C[C@H](NC(=O)[C@H](CCc1ccccc1)NC(=O)c1ccc(OC(=O)N2CCC(N3CCN(Cc4ccc(-n5c(O)nnc5-c5cc(C(C)C)c(O)cc5O)cc4)CC3)CC2)cc1)C(=O)N[C@@H](Cc1ccccc1)C(=O)N[C@@H](CC(C)C)C(=O)[C@@]1(C)CO1]

The title compound was prepared analogously using a similar procedure to that for SDC-TRAP-1006. ¹H NMR (400 MHz, DMSO-d₆) δ 11.92 (s, 1H), 9.59 (s, 1H), 9.41 (s, 1H), 8.56 (d, J=7.8 Hz, 1H), 8.21 (d, J=7.5 Hz, 1H), 7.98 (d, J=8.0 Hz, 1H), 7.92-7.88 (m, 3H), 7.33-7.04 (m, 16H), 6.77 (s, 1H), 6.26 (s, 1H), 4.56-4.00 (m, 4H), 3.43 (s, 2H), 3.14-0.75 (m, 53H).

ESMS calc'd for C₆₉H₈₆N₁₀O₁₁: 1230.7; found: 1231.2 (M+H)⁺.

### Example 1e

### 4-(((S)-1-(((S)-4-methyl-1-(((S)-1-(((S)-4-methyl-1-((R)-2-methyloxiran-2-yl)-1-oxopentan-2-yl)amino)-1-oxo-3-phenylpropan-2-yl)amino)-1-oxopentan-2-yl)amino)-1-oxo-4-phenylbutan-2-yl)carbamoyl)phenyl 4-(4-(3-(2,4-dihydroxy-5-isopropylphenyl)-5-(ethylcarbamoyl)-4H-1,2,4-triazol-4-yl)phenoxy)piperidine-1-carboxylate (SDC-TRAP-1008)

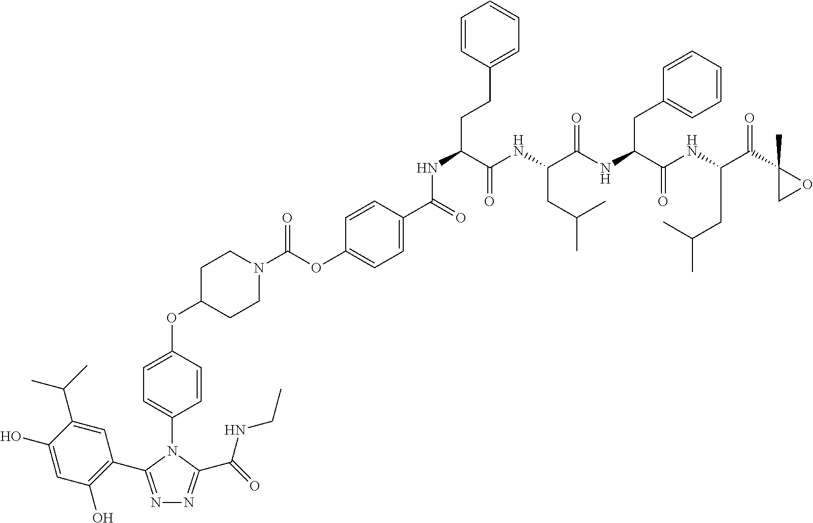 [SMILES: CCNC(=O)c1nnc(-c2cc(C(C)C)c(O)cc2O)n1-c1ccc(OC2CCN(C(=O)Oc3ccc(C(=O)N[C@@H](CCc4ccccc4)C(=O)N[C@@H](CC(C)C)C(=O)N[C@@H](Cc4ccccc4)C(=O)N[C@@H](CC(C)C)C(=O)[C@@]4(C)CO4)cc3)CC2)cc1]

The title compound was prepared analogously using a similar procedure to that for SDC-TRAP-1006. ¹H NMR (400 MHz, DMSO-d₆) δ 10.68 (s, 1H), 9.77 (s, 1H), 8.95 (t, J=5.8 Hz, 1H), 8.57 (d, J=7.7 Hz, 1H), 8.21 (d, J=7.5 Hz, 1H), 7.98 (d, J=8.0 Hz, 1H), 7.92-7.88 (m, 3H), 7.30-7.07 (m, 16H), 6.60 (s, 1H), 6.35 (s, 1H), 4.75-3.70 (m, 6H), 3.21-0.75 (m, 48H). ESMS calc'd for C₆₇H₈₁N₉O₁₂: 1203.6; found: 1204.2 (M+H)⁺.

### Example 1f

### 4-(((S)-1-(((S)-4-methyl-1-(((S)-1-(((S)-4-methyl-1-((R)-2-methyloxiran-2-yl)-1-oxopentan-2-yl)amino)-1-oxo-3-phenylpropan-2-yl)amino)-1-oxopentan-2-yl)amino)-1-oxo-4-phenylbutan-2-yl)carbamoyl)phenyl (1-(1-(4-(3-(2,4-dihydroxy-5-isopropylphenyl)-5-(ethylcarbamoyl)-4H-1,2,4-triazol-4-yl)benzyl)piperidine-4-carbonyl)piperidin-4-yl)(methyl)carbamate (SDC-TRAP-1009)

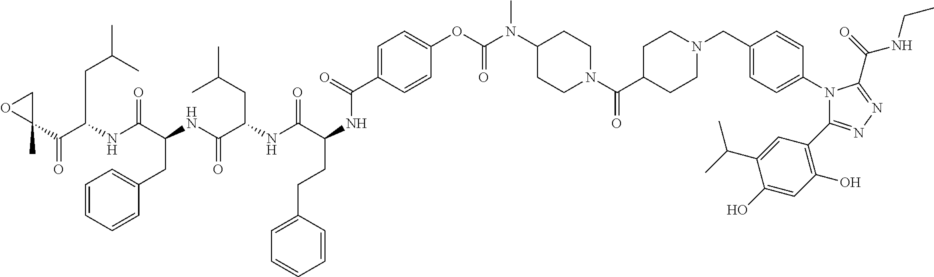 [SMILES: CCNC(=O)c1nnc(-c2cc(C(C)C)c(O)cc2O)n1-c1ccc(CN2CCC(C(=O)N3CCC(N(C)C(=O)Oc4ccc(C(=O)N[C@@H](CCc5ccccc5)C(=O)N[C@@H](CC(C)C)C(=O)N[C@@H](Cc5ccccc5)C(=O)N[C@@H](CC(C)C)C(=O)[C@@]5(C)CO5)cc4)CC3)CC2)cc1]

The title compound was prepared analogously using a similar procedure to that for SDC-TRAP-1006. ¹H NMR (400 MHz, DMSO-d₆) δ 10.62 (s, 1H), 9.76 (s, 1H), 8.96 (t, J=6.0 Hz, 1H), 8.56 (d, J=7.5 Hz, 1H), 8.21 (d, J=7.5 Hz, 1H), 7.98 (d, J=8.0 Hz, 1H), 7.94-7.85 (m, 3H), 7.38 (d, J=8.2 Hz, 2H), 7.34-7.05 (m, 14H), 6.58 (s, 1H), 6.34 (s, 1H), 4.58-4.00 (m, 4H), 3.49 (s, 2H), 3.22-0.75 (m, 62H). ESMS calc'd for C₇₅H₉₅N₁₁O₁₂: 1341.7; found: 1342.5 (M+H)⁺.

### Example 1g

### 4-((4S,7S,10S,13S)-10-benzyl-7-isobutyl-15-methyl-13-((R)-2-methyloxirane-2-carbonyl)-2,5,8,11-tetraoxo-4-phenethyl-3,6,9,12-tetraazahexadecyl)phenyl 4-(4-(3-(2,4-dihydroxy-5-isopropylphenyl)-5-hydroxy-4H-1,2,4-triazol-4-yl)benzyl)piperazine-1-carboxylate (SDC-TRAP-1010)

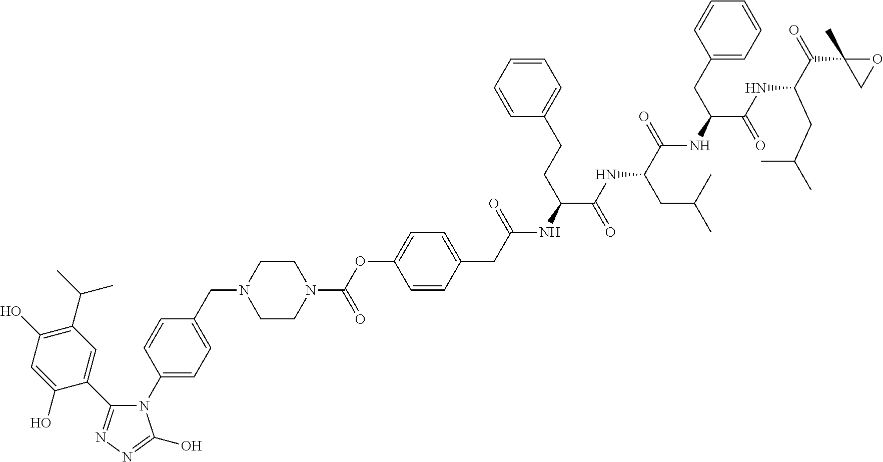 [SMILES: CC(C)C[C@H](NC(=O)[C@H](CCc1ccccc1)NC(=O)Cc1ccc(OC(=O)N2CCN(Cc3ccc(-n4c(O)nnc4-c4cc(C(C)C)c(O)cc4O)cc3)CC2)cc1)C(=O)N[C@@H](Cc1ccccc1)C(=O)N[C@@H](CC(C)C)C(=O)[C@@]1(C)CO1]

The title compound was prepared analogously using a similar procedure to that for SDC-TRAP-1006. ¹H NMR (400 MHz, DMSO-d₆) δ 11.97 (s, 1H), 9.61 (s, 1H), 9.33 (s, 1H), 8.35 (d, J=8.0 Hz, 1H), 8.24 (d, J=7.4 Hz, 1H), 7.99 (d, J=8.2 Hz, 1H), 7.85 (d, J=8.2 Hz, 1H), 7.48 (brs, 2H), 7.33-7.22 (m, 6H), 7.24-7.01 (m, 10H), 6.89 (s, 1H), 6.25 (s, 1H), 4.57-4.18 (m, 4H), 3.60-0.74 (m, 48H). ESMS calc'd for C₆₅H₇₉N₉O₁₁: 1161.6; found: 1162.2 (M+H)⁺.

### Example 1h

### 4-((4S,7S,10S,13S)-10-benzyl-7-isobutyl-15-methyl-13-((R)-2-methyloxirane-2-carbonyl)-2,5,8,11-tetraoxo-4-phenethyl-3,6,9,12-tetraazahexadecyl)phenyl 4-(4-(4-(3-(2,4-dihydroxy-5-isopropylphenyl)-5-hydroxy-4H-1,2,4-triazol-4-yl)benzyl)piperazin-1-yl)piperidine-1-carboxylate (SDC-TRAP-1011)

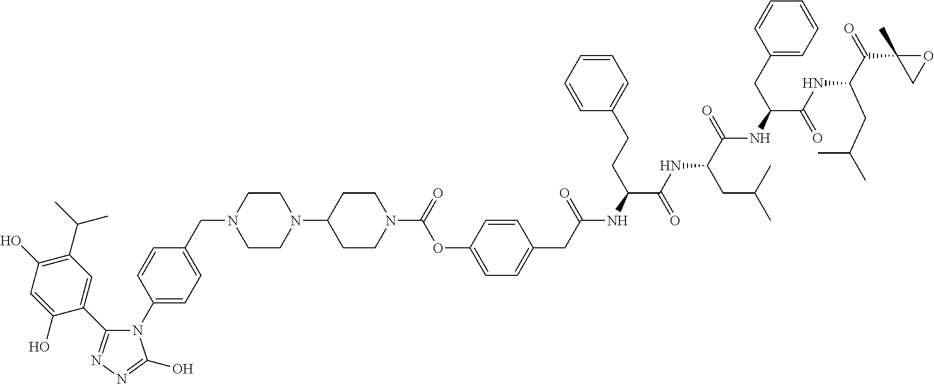 [SMILES: CC(C)C[C@H](NC(=O)[C@H](CCc1ccccc1)NC(=O)Cc1ccc(OC(=O)N2CCC(N3CCN(Cc4ccc(-n5c(O)nnc5-c5cc(C(C)C)c(O)cc5O)cc4)CC3)CC2)cc1)C(=O)N[C@@H](Cc1ccccc1)C(=O)N[C@@H](CC(C)C)C(=O)[C@@]1(C)CO1]

The title compound was prepared analogously using a similar procedure to that for SDC-TRAP-1006. ¹H NMR (400 MHz, DMSO-d₆) δ 11.92 (s, 1H), 9.58 (s, 1H), 9.40 (s, 1H), 8.33 (d, J=7.9 Hz, 1H), 8.22 (d, J=7.4 Hz, 1H), 7.96 (d, J=8.2 Hz, 1H), 7.85 (d, J=8.3 Hz, 1H), 7.32-7.22 (m, 8H), 7.21-7.03 (m, 8H), 7.01 (d, J=8.5 Hz, 2H), 6.76 (s, 1H), 6.26 (s, 1H), 4.60-4.21 (m, 4H), 3.57-0.74 (m, 57H). ESMS calc'd for C₇₀H₈₈N₁₀O₁₁: 1244.7; found: 1245.2 (M+H)⁺.

### Example 1i

### 1-((4S,7S,10S,13S)-10-benzyl-7-isobutyl-15-methyl-13-((R)-2-methyloxirane-2-carbonyl)-2,5,8,11-tetraoxo-4-phenethyl-3,6,9,12-tetraazahexadecyl)piperidin-4-yl 1-(1-(4-(3-(2,4-dihydroxy-5-isopropylphenyl)-5-(ethylcarbamoyl)-4H-1,2,4-triazol-4-yl)benzyl)piperidine-4-carbonyl)piperidine-4-carboxylate (SDC-TRAP-1012)

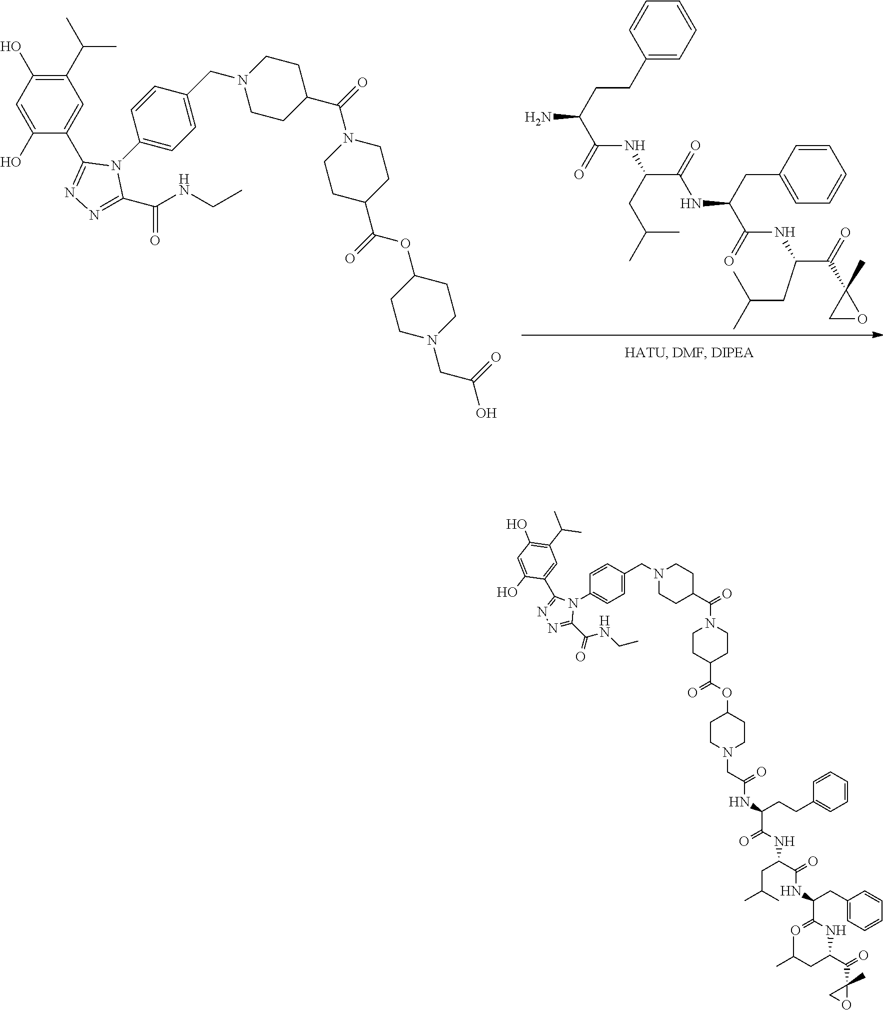 [SMILES: CCNC(=O)c1nnc(-c2cc(C(C)C)c(O)cc2O)n1-c1ccc(CN2CCC(C(=O)N3CCC(C(=O)OC4CCN(CC(=O)O)CC4)CC3)CC2)cc1 ; CC(C)C[C@H](NC(=O)[C@@H](N)CCc1ccccc1)C(=O)N[C@@H](Cc1ccccc1)C(=O)N[C@@H](CC(C)C)C(=O)[C@@]1(C)CO1 ; CCNC(=O)c1nnc(-c2cc(C(C)C)c(O)cc2O)n1-c1ccc(CN2CCC(C(=O)N3CCC(C(=O)OC4CCN(CC(=O)N[C@@H](CCc5ccccc5)C(=O)N[C@@H](CC(C)C)C(=O)N[C@@H](Cc5ccccc5)C(=O)N[C@@H](CC(C)C)C(=O)[C@@]5(C)CO5)CC4)CC3)CC2)cc1]

To a solution of 2-(4-((1-(1-(4-(3-(2,4-dihydroxy-5-isopropylphenyl)-5-(ethylcarbamoyl)-4H-1,2,4-triazol-4-yl)benzyl)piperidine-4-carbonyl)piperidine-4-carbonyl)oxy)piperidin-1-yl)acetic acid (233 mg, 0.307 mmol) and (S)-2-((S)-2-amino-4-phenylbutanamido)-4-methyl-N—((S)-1-(((S)-4-methyl-1-((R)-2-methyloxiran-2-yl)-1-oxopentan-2-yl)amino)-1-oxo-3-phenylpropan-2-yl)pentanamide (182 mg, 0.307 mmol) in DMF (7.0 mL) was added HATU (117 mg, 0.307 mmol) and DIPEA (0.7 mL). The mixture was stirred at room temperature for 3 hrs. Solvent was evaporated under reduced pressure to give a residue, which was purified by ISCO over silica gel to afford the title compound (SDC-TRAP-1012) (88 mg) as a white solid. ¹H NMR (400 MHz, DMSO-d₆) δ 10.62 (s, 1H), 9.76 (s, 1H), 8.96 (t, J=6.0 Hz, 1H), 8.23 (d, J=7.4 Hz, 1H), 8.09 (d, J=8.0 Hz, 1H), 7.91 (dd, J=25.7, 8.3 Hz, 2H), 7.37 (d, J=8.2 Hz, 2H), 7.28 (dd, J=14.0, 7.7 Hz, 4H), 7.22-7.05 (m, 8H), 6.57 (s, 1H), 6.34 (s, 1H), 4.76-4.26 (m, 5H), 3.48 (s, 2H), 3.22-0.76 (m, 69H). ESMS calc'd for C₇₄H₉₉N₁₁O₁₂: 1333.8; found: 1335.5 (M+H)⁺.

### Example 1j

### 1-((4S,7S,10S,13S)-10-benzyl-7-isobutyl-15-methyl-13-((R)-2-methyloxirane-2-carbonyl)-2,5,8,11-tetraoxo-4-phenethyl-3,6,9,12-tetraazahexadecyl)piperidin-4-yl 4-(4-(2-(5-(3-(2,4-dihydroxy-5-isopropylphenyl)-5-hydroxy-4H-1,2,4-triazol-4-yl)-1H-indol-1-yl)ethyl)-[1,4′-bipiperidin]-1′-yl)-4-oxobutanoate (SDC-TRAP-1013)

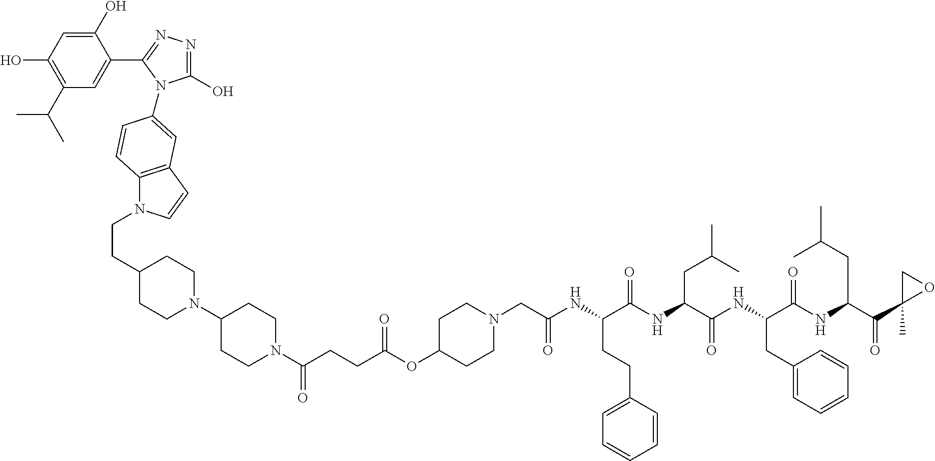 [SMILES: CC(C)C[C@H](NC(=O)[C@H](CCc1ccccc1)NC(=O)CN1CCC(OC(=O)CCC(=O)N2CCC(N3CCC(CCn4ccc5cc(-n6c(O)nnc6-c6cc(C(C)C)c(O)cc6O)ccc54)CC3)CC2)CC1)C(=O)N[C@@H](Cc1ccccc1)C(=O)N[C@@H](CC(C)C)C(=O)[C@@]1(C)CO1]

The title compound was prepared analogously using a similar procedure to that for SDC-TRAP-1012. ESMS calc'd for C₇₆H₁₀₁N₁₁O₁₂: 1359.8; found: 1360.7 (M+H)⁺.

### Example 1k

### 1-((4S,7S,10S,13S)-10-benzyl-7-isobutyl-15-methyl-13-((R)-2-methyloxirane-2-carbonyl)-2,5,8,11-tetraoxo-4-phenethyl-3,6,9,12-tetraazahexadecyl)piperidin-4-yl 4-(4-(4-(4-(3-(2,4-dihydroxy-5-isopropylphenyl)-5-hydroxy-4H-1,2,4-triazol-4-yl)benzyl)piperazin-1-yl)piperidin-1-yl)-4-oxobutanoate (SDC-TRAP-1014)

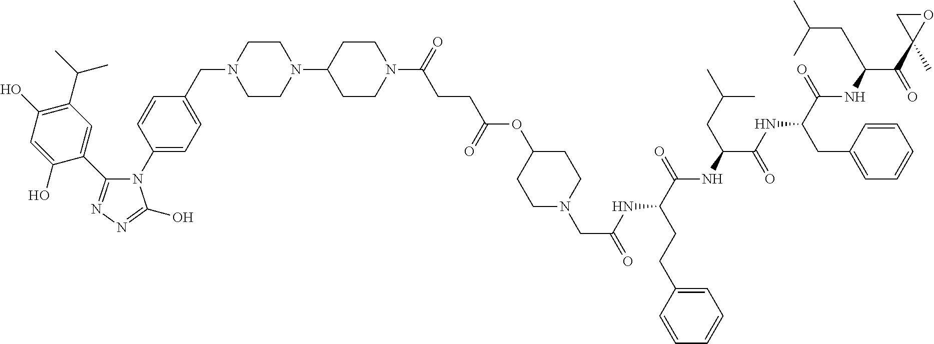 [SMILES: CC(C)C[C@H](NC(=O)[C@H](CCc1ccccc1)NC(=O)CN1CCC(OC(=O)CCC(=O)N2CCC(N3CCN(Cc4ccc(-n5c(O)nnc5-c5cc(C(C)C)c(O)cc5O)cc4)CC3)CC2)CC1)C(=O)N[C@@H](Cc1ccccc1)C(=O)N[C@@H](CC(C)C)C(=O)[C@@]1(C)CO1]

The title compound was prepared analogously using a similar procedure to that for SDC-TRAP-1012. ¹H NMR (400 MHz, DMSO-d₆) δ 11.93 (s, 1H), 9.59 (s, 1H), 9.41 (s, 1H), 8.23 (d, J=7.3 Hz, 1H), 8.09 (d, J=8.0 Hz, 1H), 7.95 (d, J=8.4 Hz, 1H), 7.87 (d, J=8.0 Hz, 1H), 7.29-7.05 (m, 14H), 6.76 (s, 1H), 6.26 (s, 1H), 4.70-4.25 (m, 5H), 3.85 (brs, 1H), 3.60 (brs, 1H), 3.41 (s, 2H), 3.01-0.76 (m, 65H). ESMS calc'd for C₇₂H₉₇N₁₁O₁₂: 1307.7; found: 1308.3 (M+H)⁺.

### Example 1l

### 1-((4S,7S,10S,13S)-10-benzyl-7-isobutyl-15-methyl-13-((R)-2-methyloxirane-2-carbonyl)-2,5,8,11-tetraoxo-4-phenethyl-3,6,9,12-tetraazahexadecyl)piperidin-4-yl 4-((1-(1-(4-(3-(2,4-dihydroxy-5-isopropylphenyl)-5-(ethylcarbamoyl)-4H-1,2,4-triazol-4-yl)benzyl)piperidine-4-carbonyl)piperidin-4-yl)(methyl)amino)-4-oxobutanoate (SDC-TRAP-1015)

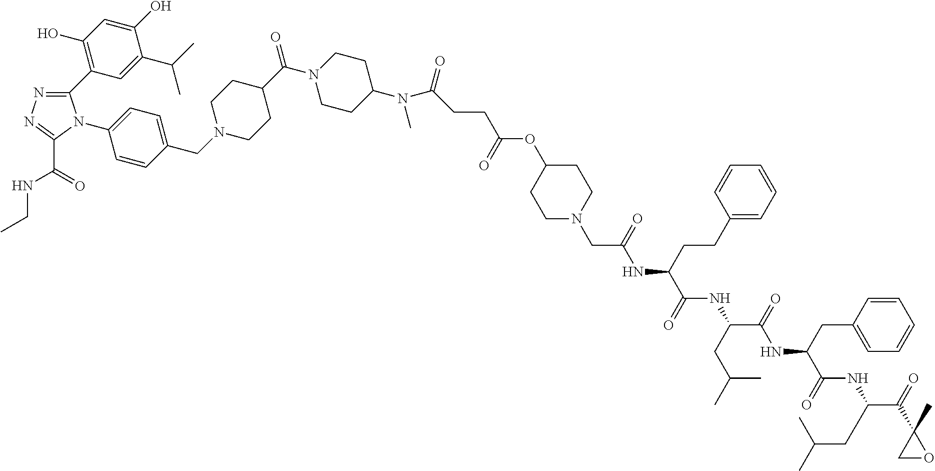 [SMILES: CCNC(=O)c1nnc(-c2cc(C(C)C)c(O)cc2O)n1-c1ccc(CN2CCC(C(=O)N3CCC(N(C)C(=O)CCC(=O)OC4CCN(CC(=O)N[C@@H](CCc5ccccc5)C(=O)N[C@@H](CC(C)C)C(=O)N[C@@H](Cc5ccccc5)C(=O)N[C@@H](CC(C)C)C(=O)[C@@]5(C)CO5)CC4)CC3)CC2)cc1]

The title compound was prepared analogously using a similar procedure to that for SDC-TRAP-1012. ESMS calc'd for C₇₈H₁₀₆N₁₂O₁₃: 1418.8; found: 1419.4 (M+H)⁺.

### Example 1m

### 1-((4S,7S,10S,13S)-10-benzyl-7-isobutyl-15-methyl-13-((R)-2-methyloxirane-2-carbonyl)-2,5,8,11-tetraoxo-4-phenethyl-3,6,9,12-tetraazahexadecyl)piperidin-4-yl 4-(4-(4-(4-(3-(2,4-dihydroxy-5-isopropylphenyl)-5-hydroxy-4H-1,2,4-triazol-4-yl)benzyl)piperazine-1-carbonyl)piperidin-1-yl)-4-oxobutanoate (SDC-TRAP-1016)

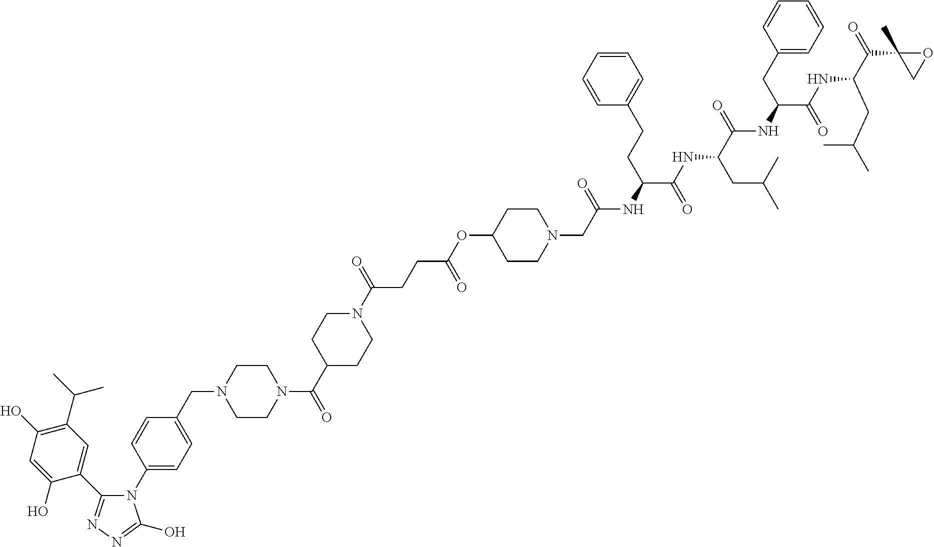 [SMILES: CC(C)C[C@H](NC(=O)[C@H](CCc1ccccc1)NC(=O)CN1CCC(OC(=O)CCC(=O)N2CCC(C(=O)N3CCN(Cc4ccc(-n5c(O)nnc5-c5cc(C(C)C)c(O)cc5O)cc4)CC3)CC2)CC1)C(=O)N[C@@H](Cc1ccccc1)C(=O)N[C@@H](CC(C)C)C(=O)[C@@]1(C)CO1]

The title compound was prepared analogously using a similar procedure to that for SDC-TRAP-1012. ¹H NMR (400 MHz, DMSO-d₆) δ 11.93 (s, 1H), 9.59 (s, 1H), 9.40 (s, 1H), 8.22 (d, J=7.4 Hz, 1H), 8.08 (d, J=8.2 Hz, 1H), 7.94 (d, J=8.4 Hz, 1H), 7.88 (d, J=8.0 Hz, 1H), 7.34-7.05 (m, 14H), 6.78 (s, 1H), 6.26 (s, 1H), 4.70-4.24 (m, 5H), 3.86 (d, J=13.1 Hz, 2H), 3.51-0.76 (m, 67H). ESMS calc'd for C₇₃H₉₇N₁₁O₁₃: 1335.7; found: 1336.3 (M+H)⁺.

### Typical Procedure for Carfilzomib-Hsp90i Conjugate

### Example 1n

### 1-(4-(3-(2,4-dihydroxy-5-isopropylphenyl)-5-(ethylcarbamoyl)-4H-1,2,4-triazol-4-yl)benzyl)-N—((S)-1-(((S)-4-methyl-1-(((S)-1-(((S)-4-methyl-1-((R)-2-methyloxiran-2-yl)-1-oxopentan-2-yl)amino)-1-oxo-3-phenylpropan-2-yl)amino)-1-oxopentan-2-yl)amino)-1-oxo-4-phenylbutan-2-yl)piperidine-4-carboxamide

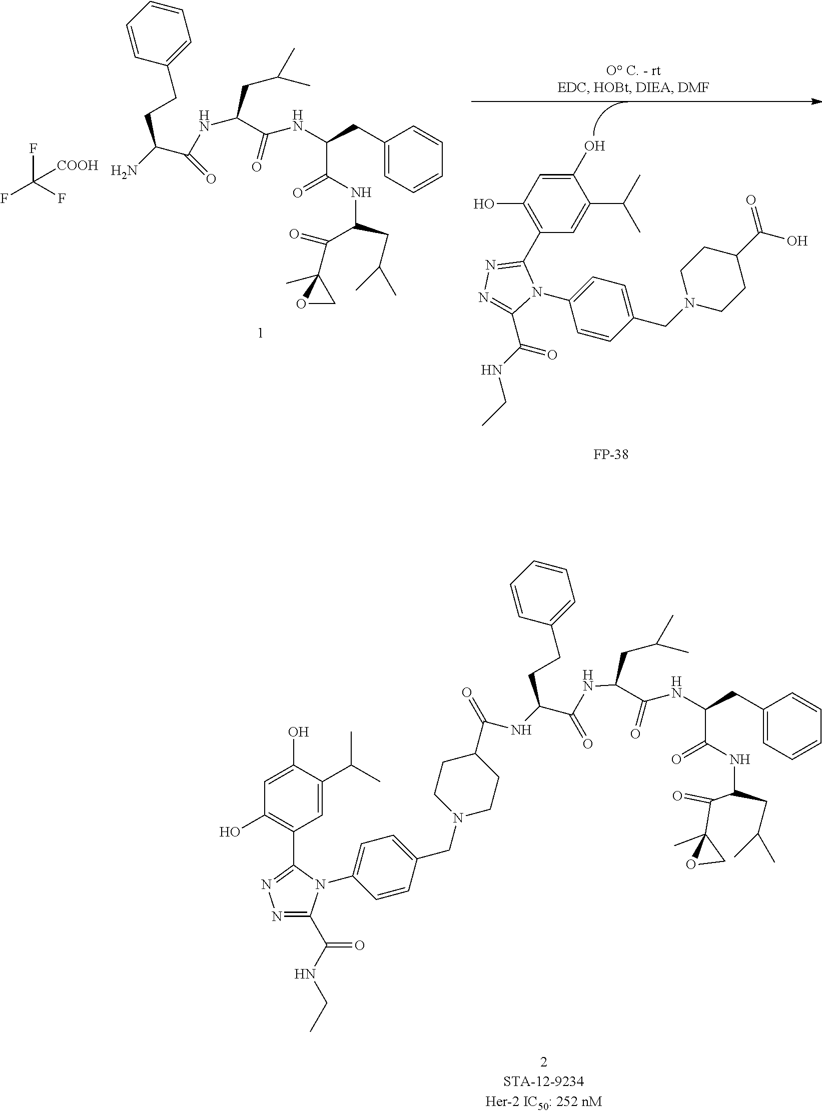

To a stirred solution of 1-(4-(3-(2,4-dihydroxy-5-isopropylphenyl)-5-(ethylcarbamoyl)-4H-1,2,4-triazol-4-yl)benzyl)piperidine-4-carboxylic acid (FP-38, 20 mg, 0.04 mmol) in EtOAc/DMF (1:1/v:v, 10 mL) was added EDC (8.2 mg, 0.04 mmol), HOBt (6.1 mg, 0.04 mmol), and DIEA (0.3 ml) at 0° C. Stirring was continued at 0° C. for half an hour. (S)-2-((S)-2-amino-4-phenylbutanamido)-4-methyl-N—((S)-1-(((S)-4-methyl-1-((R)-2-methyloxiran-2-yl)-1-oxopentan-2-yl)amino)-1-oxo-3-phenylpropan-2-yl)pentanamide 2,2,2-trifluoroacetate (1, Lot# GLS-JSH-102513, 21.2 mg, 0.03 mmol) was then added in one portion followed by DIEA (0.2 mL). The stirring was continued at 0° C. for 2 h and room temperature for 12 h. The reaction mixture was diluted with EtOAc (20 mL), washed successively with H₂O (30 mL), Sat. NaHCO₃ (2×15 mL), H₂O (15 mL), 0.5 N HCL (2×15 mL), H₂O (15 mL), and brine (15 mL) and dried over anhydrous Na₂SO₄. Removal of solvent provided a white solid. After a brief purification on ISCO column (20 g preloaded column) using a mixture of DCM/MeOH as eluent afforded the final product, 1-(4-(3-(2,4-dihydroxy-5-isopropylphenyl)-5-(ethylcarbamoyl)-4H-1,2,4-triazol-4-yl)benzyl)-N—((S)-1-(((S)-4-methyl-1-(((S)-1-(((S)-4-methyl-1-((R)-2-methyloxiran-2-yl)-1-oxopentan-2-yl)amino)-1-oxo-3-phenylpropan-2-yl)amino)-1-oxopentan-2-yl)amino)-1-oxo-4-phenylbutan-2-yl)piperidine-4-carboxamide, as a white solid (8 mg, 25% yield). LC/MS calc'd for C₆₁H₁₇₉N₉O₉ (1081.4); found: 1082.4 (M+1). ¹H NMR (400 MHz, Chloroform-d) δ 8.18 (s, 1H), 7.49-7.61 (m, 2H), 7.48-7.16 (m, 18H), 7.01 (s, 1H), 6.48 (s, 1H), 4.45-4.13 (m, 5H), 4.01-3.80 (m, 2H), 3.75-3.60 (m, 3H), 3.50-3.25 (m, 3H), 3.24-1.18 (m, 25H), 0.88-0.75 (m, 18H).

Other SDC-TRAP compounds with an amide linker (examples 2 through 12) were prepared in a similar manner as described above:

### Example 1o

### 4-(4-(((2S,5S,8S,11S,14S)-11-benzyl-8-isobutyl-16-methyl-14-((R)-2-methyloxirane-2-carbonyl)-3,6,9,12-tetraoxo-5-phenethyl-4,7,10,13-tetraazaheptadecan-2-yl)carbamoyl)phenyl)-5-(2,4-dihydroxy-5-isopropylphenyl)-N-ethyl-4H-1,2,4-triazole-3-carboxamide

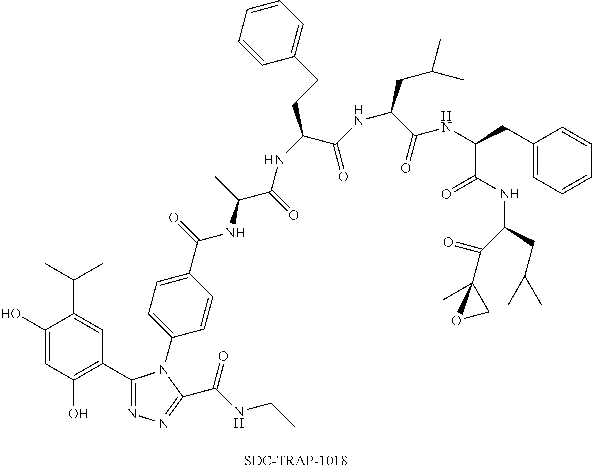 [SMILES: CCNC(=O)c1nnc(-c2cc(C(C)C)c(O)cc2O)n1-c1ccc(C(=O)N[C@@H](C)C(=O)N[C@@H](CCc2ccccc2)C(=O)N[C@@H](CC(C)C)C(=O)N[C@@H](Cc2ccccc2)C(=O)N[C@@H](CC(C)C)C(=O)[C@@]2(C)CO2)cc1]

LC/MS calc'd for C₅₈H₇₃N₉O₁₀ (1055.6); found: 1078.2 (M+Na). ¹H NMR (400 MHz, Chloroform-d) δ 7.55 (d, J=8.1 Hz, 1H), 7.51 (s, 1H), 7.31 (t, J=7.4 Hz, 2H), 7.28-7.14 (m, 16H), 7.01 (s, 1H), 6.70 (d, J=7.8 Hz, 1H), 6.48 (s, 1H), 6.37 (d, J=7.1 Hz, 1H), 4.61-4.50 (m, 3H), 4.38-4.23 (m, 3H), 4.19-4.13 (m, 2H), 3.40-2.61 (m, 12H), 2.33-1.18 (m, 11H), 0.99-0.81 (m, 18H).

### Example 1p

### 1-(4-(3-(2,4-dihydroxy-5-isopropylphenyl)-5-hydroxy-4H-1,2,4-triazol-4-yl)benzyl)-N—((S)-1-(((S)-4-methyl-1-(((S)-1-(((S)-4-methyl-1-((R)-2-methyloxiran-2-yl)-1-oxopentan-2-yl)amino)-1-oxo-3-phenylpropan-2-yl)amino)-1-oxopentan-2-yl)amino)-1-oxo-4-phenylbutan-2-yl)piperidine-4-carboxamide

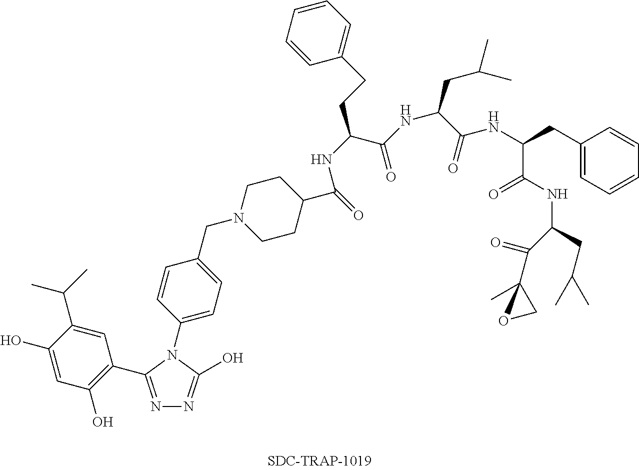 [SMILES: CC(C)C[C@H](NC(=O)[C@H](CCc1ccccc1)NC(=O)C1CCN(Cc2ccc(-n3c(O)nnc3-c3cc(C(C)C)c(O)cc3O)cc2)CC1)C(=O)N[C@@H](Cc1ccccc1)C(=O)N[C@@H](CC(C)C)C(=O)[C@@]1(C)CO1]

LC/MS calc'd for C₅₈H₇₄N₈O₉ (1026.6); found: 1027.3 (M+1). ¹H NMR (400 MHz, Chloroform-d) δ 11.65 (s, 1H), 9.45 (s, 1H), 9.12 (s, 1H), 7.77 (s, 2H), 7.50-7.057 (m, 16H), 6.49 (s, 2H), 4.65-4.08 (m, 6H), 3.80-1.13 (m, 27H), 0.98-0.81 (, 18H).

### Example 1q

### 5-(2,4-dihydroxy-5-isopropylphenyl)-N-ethyl-4-(4-(((S)-1-(((S)-4-methyl-1-(((S)-1-(((S)-4-methyl-1-((R)-2-methyloxiran-2-yl)-1-oxopentan-2-yl)amino)-1-oxo-3-phenylpropan-2-yl)amino)-1-oxopentan-2-yl)amino)-1-oxo-4-phenylbutan-2-yl)carbamoyl)phenyl)-4H-1,2,4-triazole-3-carboxamide

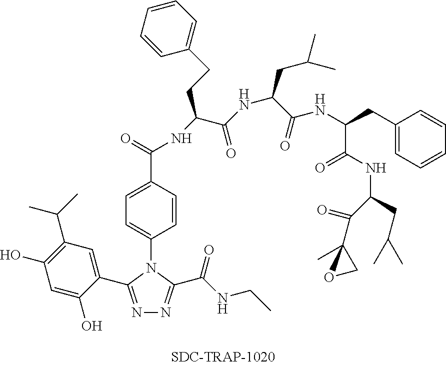 [SMILES: CCNC(=O)c1nnc(-c2cc(C(C)C)c(O)cc2O)n1-c1ccc(C(=O)N[C@@H](CCc2ccccc2)C(=O)N[C@@H](CC(C)C)C(=O)N[C@@H](Cc2ccccc2)C(=O)N[C@@H](CC(C)C)C(=O)[C@@]2(C)CO2)cc1]

LC/MS calc'd for C55H68N8O9 (984.5); found: 985.1 (M+1). ¹H NMR (400 MHz, Chloroform-d) δ 9.25 (s, 1H), 8.01 (dd, J=7.9, 3.0 Hz, 2H), 7.87 (d, J=7.3 Hz, 1H), 7.70 (s, 1H), 7.44 (dd, J=8.5, 3.1 Hz, 2H), 7.39-7.00 (m, 14H), 6.53 (d, J=3.2 Hz, 1H), 6.35 (d, J=3.2 Hz, 1H), 4.67 (d, J=7.6 Hz, 1H), 4.55 (s, 2H), 4.35 (s, 1H), 3.36-1.20 (m, 23H), 0.93-0.81 (m, 12H), 0.71 (dd, J=6.8, 3.2 Hz, 6H).

### Example 1 r

### 4-(4-(4-(3-(2,4-dihydroxy-5-isopropylphenyl)-5-hydroxy-4H-1,2,4-triazol-4-yl)benzyl)piperazin-1-yl)-N—((S)-1-(((S)-4-methyl-1-(((S)-1-(((S)-4-methyl-1-((R)-2-methyloxiran-2-yl)-1-oxopentan-2-yl)amino)-1-oxo-3-phenylpropan-2-yl)amino)-1-oxopentan-2-yl)amino)-1-oxo-4-phenylbutan-2-yl)cyclohexanecarboxamide

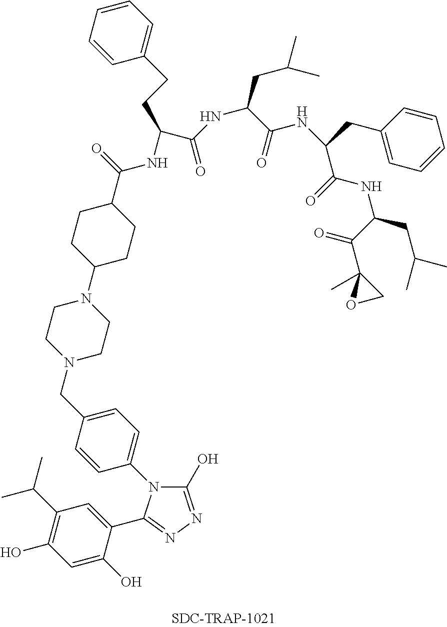 [SMILES: CC(C)C[C@H](NC(=O)[C@H](CCc1ccccc1)NC(=O)C1CCC(N2CCN(Cc3ccc(-n4c(O)nnc4-c4cc(C(C)C)c(O)cc4O)cc3)CC2)CC1)C(=O)N[C@@H](Cc1ccccc1)C(=O)N[C@@H](CC(C)C)C(=O)[C@@]1(C)CO1]

LC/MS calc'd for C63H83N9O9 (1109.6); found: 1110.3 (M+1). ¹H NMR (400 MHz, Methanol-d₄) δ 7.69 (d, J=8.3 Hz, 2H), 7.44-7.35 (m, 2H), 7.33-7.24 (m, 2H), 7.26-7.18 (m, 6H), 7.22-7.06 (m, 2H), 6.95 (s, 1H), 6.26 (s, 1H), 4.63 (ddd, J=8.7, 5.4, 3.0 Hz, 1H), 4.53 (dd, J=10.5, 3.4 Hz, 1H), 4.46 (s, 3H), 4.30 (ddd, J=28.8, 9.1, 5.6 Hz, 2H), 3.84 (s, 4H), 3.22-3.03 (m, 3H), 2.95-2.84 (m, 2H), 2.71-2.58 (m, 2H), 2.39-2.28 (m, 4H), 2.10-1.97 (m, 6H), 1.70-1.55 (m, 3H), 1.54-1.40 (m, 2H), 1.47 (s, 3H), 1.44-1.30 (m, 2H), 1.30-1.20 (m, 1H), 1.07 (d, J=6.9 Hz, 9H), 1.03-0.82 (m, 15H).

### Example 1s

### (S)-2-((S)-2-(2-(4-(4-(3-(2,4-dihydroxy-5-isopropylphenyl)-5-hydroxy-4H-1,2,4-triazol-4-yl)benzyl)piperazin-1-yl)acetamido)-4-phenylbutanamido)-4-methyl-N—((S)-1-(((S)-4-methyl-1-((R)-2-methyloxiran-2-yl)-1-oxopentan-2-yl)amino)-1-oxo-3-phenylpropan-2-yl)pentanamide

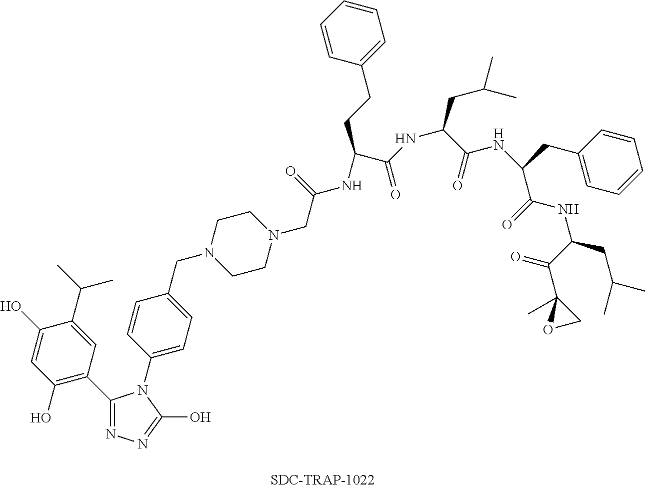 [SMILES: CC(C)C[C@H](NC(=O)[C@H](CCc1ccccc1)NC(=O)CN1CCN(Cc2ccc(-n3c(O)nnc3-c3cc(C(C)C)c(O)cc3O)cc2)CC1)C(=O)N[C@@H](Cc1ccccc1)C(=O)N[C@@H](CC(C)C)C(=O)[C@@]1(C)CO1]

LC/MS calc'd for C58H75N9O9 (1041.6); found: 1041.6 (Mt). ¹H NMR (400 MHz, Chloroform-d) δ 11.41 (s, 1H), 9.83 (d, J=6.0 Hz, 1H), 8.91 (d, J=7.0 Hz, 1H), 7.64 (t, J=6.9 Hz, 1H), 7.50-7.42 (m, 2H), 7.35-7.22 (m, 6H), 7.22-7.03 (m, 9H), 6.47 (dd, J=12.5, 5.8 Hz, 2H), 4.65 (p, J=6.9 Hz, 1H), 4.52 (dd, J=9.4, 5.6 Hz, 1H), 4.34 (s, 1H), 3.54 (d, J=5.2 Hz, 2H), 3.24 (t, J=5.5 Hz, 1H), 3.15-2.88 (m, 4H), 2.82 (t, J=5.5 Hz, 1H), 2.61 (q, J=6.7 Hz, 2H), 2.54 (s, 9H), 2.33 (s, 2H), 1.92 (dd, J=14.4, 7.6 Hz, 1H), 1.60-1.38 (m, 9H), 0.86 (ddd, J=14.0, 10.8, 5.9 Hz, 12H), 0.70 (t, J=6.3 Hz, 6H).

### Example 1t

### (S)-2-((S)-2-(2-(4-(2-(5-(3-(2,4-dihydroxy-5-isopropylphenyl)-5-hydroxy-4H-1,2,4-triazol-4-yl)-1H-indol-1-yl)ethyl)piperidin-1-yl)acetamido)-4-phenylbutanamido)-4-methyl-N—((S)-1-(((S)-4-methyl-1-((R)-2-methyloxiran-2-yl)-1-oxopentan-2-yl)amino)-1-oxo-3-phenylpropan-2-yl)pentanamide

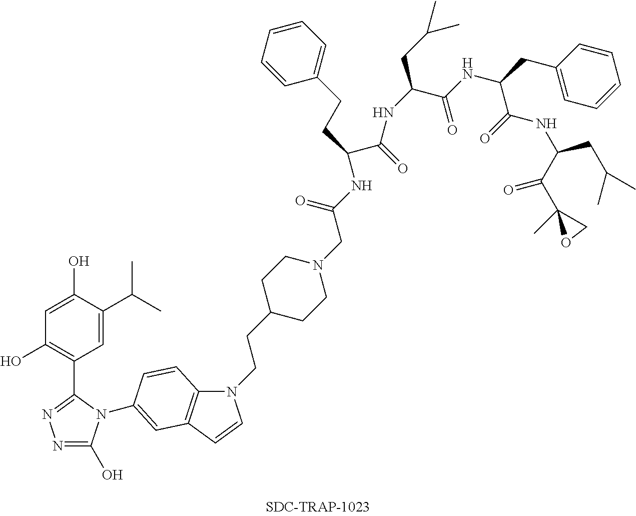 [SMILES: CC(C)C[C@H](NC(=O)[C@H](CCc1ccccc1)NC(=O)CN1CCC(CCn2ccc3cc(-n4c(O)nnc4-c4cc(C(C)C)c(O)cc4O)ccc32)CC1)C(=O)N[C@@H](Cc1ccccc1)C(=O)N[C@@H](CC(C)C)C(=O)[C@@]1(C)CO1]

LC/MS calc'd for C62H79N9O9 (1093.6); found: 1094.1 (M+1). ¹H NMR (400 MHz, Chloroform-d) δ 9.92 (s, 1H), 9.65 (s, 1H), 7.71 (d, J=8.3 Hz, 1H), 7.58 (d, J=2.0 Hz, 1H), 7.43 (d, J=8.6 Hz, 1H), 7.26 (d, J=14.7 Hz, 1H), 7.21-7.06 (m, 14H), 6.53-6.45 (m, 2H), 6.38 (s, 1H), 6.01 (s, 1H), 4.65-4.15 (m, 10H), 3.50-2.60 (m, 8H), 2.25-0.75 (m, 34H), 0.54 (d, J=6.9 Hz, 3H).

### Example 1u

### (S)-2-((S)-2-(2-(4-(4-(3-(2,4-dihydroxy-5-isopropylphenyl)-5-hydroxy-4H-1,2,4-triazol-4-yl)phenyl)piperazin-1-yl)acetamido)-4-phenylbutanamido)-4-methyl-N—((S)-1-(((S)-4-methyl-1-((R)-2-methyloxiran-2-yl)-1-oxopentan-2-yl)amino)-1-oxo-3-phenylpropan-2-yl)pentanamide

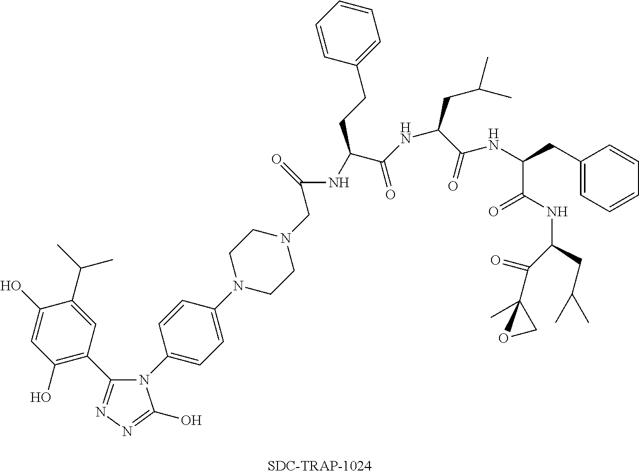 [SMILES: CC(C)C[C@H](NC(=O)[C@H](CCc1ccccc1)NC(=O)CN1CCN(c2ccc(-n3c(O)nnc3-c3cc(C(C)C)c(O)cc3O)cc2)CC1)C(=O)N[C@@H](Cc1ccccc1)C(=O)N[C@@H](CC(C)C)C(=O)[C@@]1(C)CO1]

LC/MS calc'd for C57H73N9O9 (1027.6); found: 1028.1 (M+1). ¹H NMR (400 MHz, Chloroform-d) δ 11.56 (s, 1H), 9.95 (d, J=0.8 Hz, 1H), 9.04 (s, 1H), 7.63 (d, J=8.2 Hz, 1H), 7.39 (d, J=0.9 Hz, 4H), 7.32-6.95 (m, 13H), 6.53 (s, 1H), 6.47 (d, J=1.0 Hz, 1H), 4.75-4.64 (m, 1H), 4.55-4.42 (m, 2H), 4.41-4.31 (m, 1H), 3.79-3.69 (m, 4H), 3.25-2.55 (m, 20H), 2.21-0.76 (m, 19H), 0.77 (dd, J=6.9, 1.3 Hz, 3H).

### Example 1v

### (S)-2-((S)-2-(3-(2-(4-(4-(3-(2,4-dihydroxy-5-isopropylphenyl)-5-hydroxy-4H-1,2,4-triazol-4-yl)benzyl)piperazin-1-yl)acetamido)propanamido)-4-phenylbutanamido)-4-methyl-N—((S)-1-(((S)-4-methyl-1-((R)-2-methyloxiran-2-yl)-1-oxopentan-2-yl)amino)-1-oxo-3-phenylpropan-2-yl)pentanamide

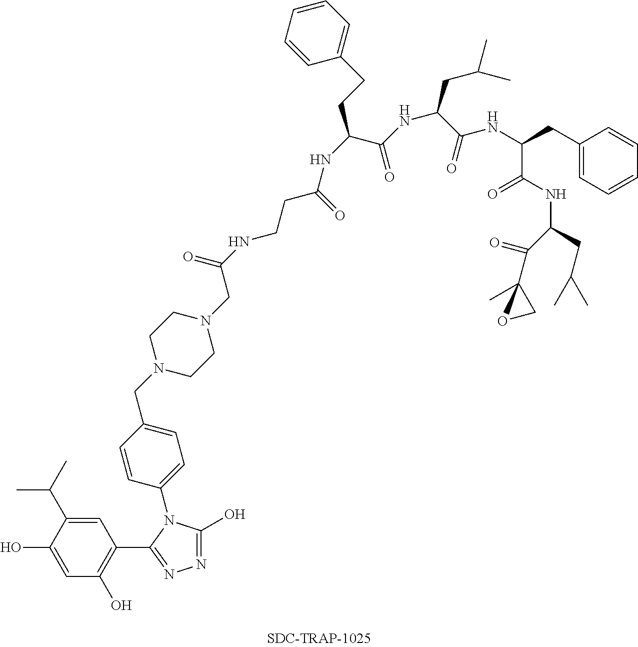 [SMILES: CC(C)C[C@H](NC(=O)[C@H](CCc1ccccc1)NC(=O)CCNC(=O)CN1CCN(Cc2ccc(-n3c(O)nnc3-c3cc(C(C)C)c(O)cc3O)cc2)CC1)C(=O)N[C@@H](Cc1ccccc1)C(=O)N[C@@H](CC(C)C)C(=O)[C@@]1(C)CO1]

LC/MS calc'd for Chemical Formula: C61H80N10O10 (1112.6); found: 1113.0 (M+1). ¹H NMR (400 MHz, Chloroform-d) δ 11.63 (s, 1H), 9.81 (d, J=4.7 Hz, 1H), 9.04 (s, 1H), 7.75 (d, J=6.1 Hz, 1H), 7.60 (s, 1H), 7.50-7.04 (m, 17H), 6.48 (dd, J=9.6, 4.8 Hz, 2H), 4.70 (s, 1H), 4.53 (s, 1H), 4.41 (s, 2H), 3.85 (s, 1H), 3.58 (d, J=4.8 Hz, 3H), 3.50-3.28 (m, 1H), 3.3-0.83 (m, 41H), 0.71 (dt, J=6.6, 4.7 Hz, 6H).

### Example 1w

### 5-(4-(4-(3-(2,4-dihydroxy-5-isopropylphenyl)-5-(ethylcarbamoyl)-4H-1,2,4-triazol-4-yl)phenoxy)piperidin-1-yl)-N—((S)-1-(((S)-4-methyl-1-(((S)-1-(((S)-4-methyl-1-((R)-2-methyloxiran-2-yl)-1-oxopentan-2-yl)amino)-1-oxo-3-phenylpropan-2-yl)amino)-1-oxopentan-2-yl)amino)-1-oxo-4-phenylbutan-2-yl)pyrazine-2-carboxamide

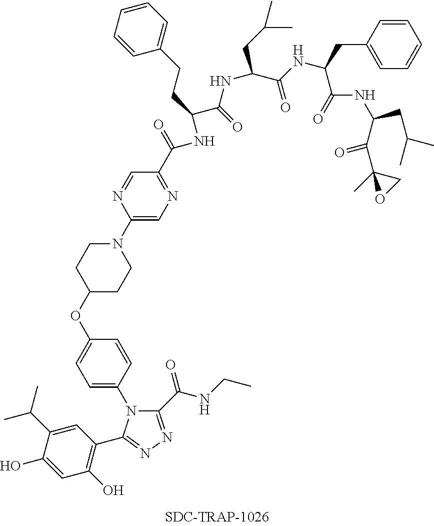 [SMILES: CCNC(=O)c1nnc(-c2cc(C(C)C)c(O)cc2O)n1-c1ccc(OC2CCN(c3cnc(C(=O)N[C@@H](CCc4ccccc4)C(=O)N[C@@H](CC(C)C)C(=O)N[C@@H](Cc4ccccc4)C(=O)N[C@@H](CC(C)C)C(=O)[C@@]4(C)CO4)cn3)CC2)cc1]

LC/MS calc'd for C64H79N11O10 (1161.6); found: 1162.3 (M+1). ¹H NMR (400 MHz, Chloroform-d) δ 11.59 (s, 1H), 8.83 (d, J=1.3 Hz, 1H), 7.85 (d, J=6.6 Hz, 1H), 7.38-7.11 (m, 13H), 7.13-7.06 (m, 2H), 6.87 (d, J=8.1 Hz, 1H), 6.64-6.51 (m, 3H), 6.47 (s, 1H), 6.04 (s, 1H), 5.74 (s, 1H), 4.75-4.61 (m, 2H), 4.54 (ddd, J=10.9, 7.8, 3.3 Hz, 1H), 4.43-4.25 (m, 2H), 3.96-3.86 (m, 5H), 3.72 (pd, J=6.7, 4.1 Hz, 2H), 3.52-3.35 (m, 3H), 3.32-2.65 (m, 8H), 2.25-1.88 (m, 4H), 1.61-1.18 (m, 9H), 0.93-0.74 (m, 18H).

### Example 1x

### 4-(4-(((3S,6S,9S,12S,15S)-12-benzyl-9-isobutyl-2,17-dimethyl-15-((R)-2-methyloxirane-2-carbonyl)-4,7,10,13-tetraoxo-6-phenethyl-5,8,11,14-tetraazaoctadecan-3-yl)carbamoyl)phenyl)-5-(2,4-dihydroxy-5-isopropylphenyl)-N-ethyl-4H-1,2,4-triazole-3-carboxamide

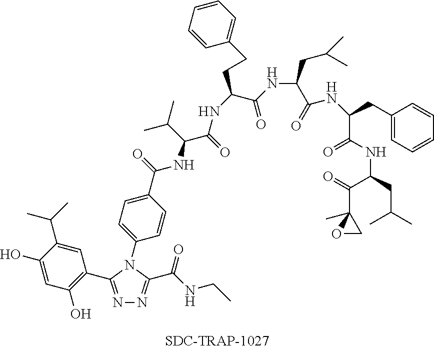 [SMILES: CCNC(=O)c1nnc(-c2cc(C(C)C)c(O)cc2O)n1-c1ccc(C(=O)N[C@H](C(=O)N[C@@H](CCc2ccccc2)C(=O)N[C@@H](CC(C)C)C(=O)N[C@@H](Cc2ccccc2)C(=O)N[C@@H](CC(C)C)C(=O)[C@@]2(C)CO2)C(C)C)cc1]

LC/MS calc'd for C60H77N9O10 (1083.6); found: 1084.1 (M+1).

### Example 1y

### 1-(1-(4-(3-(2,4-dihydroxy-5-isopropylphenyl)-5-hydroxy-4H-1,2,4-triazol-4-yl)benzyl)piperidine-4-carbonyl)-N—((S)-1-(((S)-4-methyl-1-(((S)-1-(((S)-4-methyl-1-((R)-2-methyloxiran-2-yl)-1-oxopentan-2-yl)amino)-1-oxo-3-phenylpropan-2-yl)amino)-1-oxopentan-2-yl)amino)-1-oxo-4-phenylbutan-2-yl)piperidine-4-carboxamide

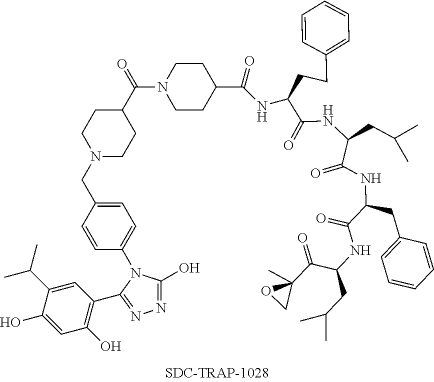 [SMILES: CC(C)C[C@H](NC(=O)[C@H](CCc1ccccc1)NC(=O)C1CCN(C(=O)C2CCN(Cc3ccc(-n4c(O)nnc4-c4cc(C(C)C)c(O)cc4O)cc3)CC2)CC1)C(=O)N[C@@H](Cc1ccccc1)C(=O)N[C@@H](CC(C)C)C(=O)[C@@]1(C)CO1]

LC/MS calc'd for C64H83N9O10 (1137.6); found: 1138.1 (M+1). ¹H NMR (400 MHz, Methanol-d₄) δ 7.82-7.53 (m, 1H), 7.39 (d, J=8.4 Hz, 2H), 7.27-6.86 (m, 11H), 6.72 (s, 1H), 6.15 (s, 1H), 4.60-4.35 (m, 3H), 4.21 (ddd, J=23.6, 9.0, 5.7 Hz, 2H), 3.88 (s, 2H), 3.25-2.75 (m, 10H), 2.73-2.67 (m, 2H), 2.67-2.39 (m, 5H), 2.15-0.62 (m, 36H).

### Example 1z

### 1-(4-(3-(2,4-dihydroxy-5-isopropylphenyl)-5-(ethylcarbamoyl)-4H-1,2,4-triazol-4-yl)benzoyl)piperidin-4-yl ((5S,8S,11S,14S)-11-benzyl-8-isobutyl-16-methyl-14-((R)-2-methyloxirane-2-carbonyl)-3,6,9,12-tetraoxo-5-phenethyl-4,7,10,13-tetraazaheptadecyl)carbamate

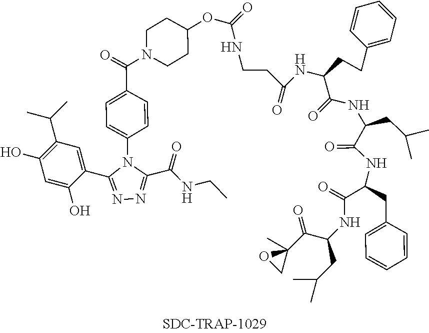 [SMILES: CCNC(=O)c1nnc(-c2cc(C(C)C)c(O)cc2O)n1-c1ccc(C(=O)N2CCC(OC(=O)NCCC(=O)N[C@@H](CCc3ccccc3)C(=O)N[C@@H](CC(C)C)C(=O)N[C@@H](Cc3ccccc3)C(=O)N[C@@H](CC(C)C)C(=O)[C@@]3(C)CO3)CC2)cc1]

General Procedure:

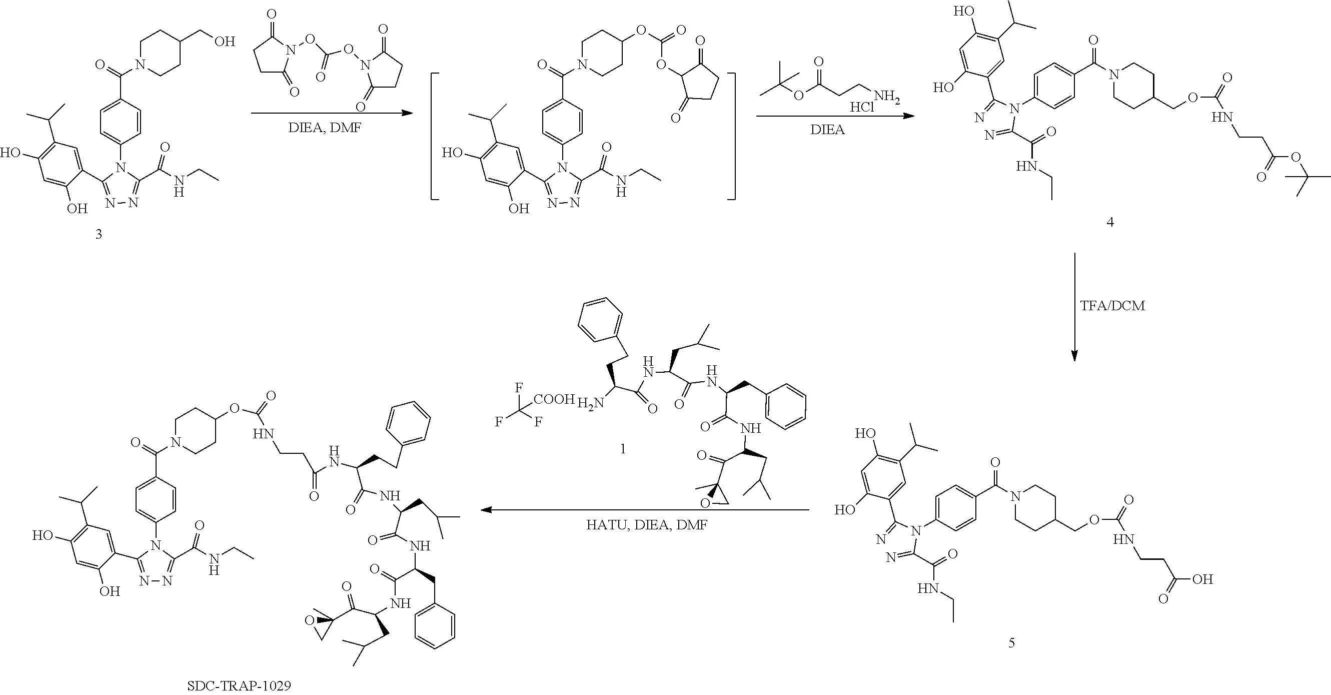

### Preparation of tert-butyl 3-((((1-(4-(3-(2,4-dihydroxy-5-isopropylphenyl)-5-(ethylcarbamoyl)-4H-1,2,4-triazol-4-yl)benzoyl)piperidin-4-yl)methoxy)carbonyl)amino)propanoate (4)

A solution of 5-(2,4-dihydroxy-5-isopropylphenyl)-N-ethyl-4-(4-(4-(hydroxymethyl)piperidine-1-carbonyl)phenyl)-4H-1,2,4-triazole-3-carboxamide (3, “Synta-FP-42”, 0.25 g, 0.5 mmol), bis(2,5-dioxopyrrolidin-1-yl) carbonate (0.26 g, 1 mmol), DIEA (0.28 mL, ˜1.6 mmol) in DMF (5 mL) was stirred at room temperature for 4 h. Tert-butyl 3-aminopropanoate hydrochloride (0.1 g, 0.55 mmol) and DIEA (0.2 mL) were then added at room temperature. The resultant clear solution was stirred at room temperature overnight. The reaction pot was diluted with water (40 mL) and the resulted solution was washed with DCM (2×15 mL) and ether (20 mL). The aqueous solution was then acidified with 6N HCl to pH 4-5 and extracted with DCM (4×20 mL). Combined DCM solution was dried over Na₂SO₄ and concentrated to afford the crude product 4 as a syrup. LC/MS calc'd for C35H46N6O8 (678.3); found: 679.1 (M+1). The crude product can be used directly in next step without further purification.

### Preparation of 3-((((1-(4-(3-(2,4-dihydroxy-5-isopropylphenyl)-5-(ethylcarbamoyl)-4H-1,2,4-triazol-4-yl)benzoyl)piperidin-4-yl)methoxy)carbonyl)amino)propanoic acid (5)

A solution of 4 in TFA (2 mL) and DCM (2 mL) was stirred at room temperature for 2 h. Volatile components were removed under reduced pressure to afford crude 3-((((1-(4-(3-(2,4-dihydroxy-5-isopropylphenyl)-5-(ethylcarbamoyl)-4H-1,2,4-triazol-4-yl)benzoyl)piperidin-4-yl)methoxy)carbonyl)amino)propanoic acid (5) as a syrup. Triturated with ether resulted in a white powder. It was collected by filtration and washed with ether. LC/MS calc'd for C31H38N6O8 (622.3); found: 623.1 (M+1). This material can be used directly in the next step without further purification.

### Synthesis of 1-(4-(3-(2,4-dihydroxy-5-isopropylphenyl)-5-(ethylcarbamoyl)-4H-1,2,4-triazol-4-yl)benzoyl)piperidin-4-yl ((5S,8S,11S,14S)-11-benzyl-8-isobutyl-16-methyl-14-((R)-2-methyloxirane-2-carbonyl)-3,6,9,12-tetraoxo-5-phenethyl-4,7,10,13-tetraazaheptadecyl)carbamate (SDC-TRAP-1029)

A solution of 5 (31 mg, 0.05 mmol), 1 (36 mg, 0.05 mmol), HATU (20 mg, 0.053 mmol), DIEA (0.2 mL) in DMF (2 mL) was stirred at room temperature for 12 h. Volatile components were removed on a rotavapor to afford a syrup which was triturated with DCM to afford the crude product, 1-(4-(3-(2,4-dihydroxy-5-isopropylphenyl)-5-(ethylcarbamoyl)-4H-1,2,4-triazol-4-yl)benzoyl)piperidin-4-yl ((5S,8S,11S,14S)-11-benzyl-8-isobutyl-16-methyl-14-((R)-2-methyloxirane-2-carbonyl)-3,6,9,12-tetraoxo-5-phenethyl-4,7,10,13-tetraazaheptadecyl)carbamate, as a solid. Further purification by column chromatography on a ISCO purification system using a mixture of DCM and MeOH as eluent to afford the pure product as a white powder (14 mg, 23% yield). LC/MS calc'd for C₆₄H₈₂N₁₀O₁₂ (1182.6); found: 1183.1 (M+1). ¹H NMR (400 MHz, Chloroform-d) δ 11.25 (s, 1H), 8.91 (s, 1H), 7.82-7.51 (m, 2H), 7.54-7.34 (m, 3H), 7.34-6.73 (m, 14H), 6.67-6.50 (m, 1H), 6.43 (s, 1H), 5.64 (s, 1H), 4.68 (d, J=7.5 Hz, 2H), 4.58 (t, J=7.5 Hz, 1H), 4.36 (s, 2H), 3.92 (s, 3H), 3.60-2.38 (m, 17H), 2.20-1.18 (m, 12H), 0.97-0.55 (m, 21H).

Other carbamate linker examples (Examples 1aa through 1ae) were synthesized similarly to the procedure described for Example 1z.

### Example 1aa

### 1-(4-(3-(2,4-dihydroxy-5-isopropylphenyl)-5-(ethylcarbamoyl)-4H-1,2,4-triazol-4-yl)benzoyl)piperidin-4-yl ((S)-1-(((S)-4-methyl-1-(((S)-1-(((S)-4-methyl-1-((R)-2-methyloxiran-2-yl)-1-oxopentan-2-yl)amino)-1-oxo-3-phenylpropan-2-yl)amino)-1-oxopentan-2-yl)amino)-1-oxo-4-phenylbutan-2-yl)carbamate

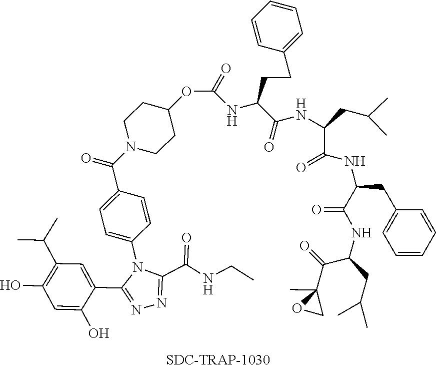 [SMILES: CCNC(=O)c1nnc(-c2cc(C(C)C)c(O)cc2O)n1-c1ccc(C(=O)N2CCC(OC(=O)N[C@@H](CCc3ccccc3)C(=O)N[C@@H](CC(C)C)C(=O)N[C@@H](Cc3ccccc3)C(=O)N[C@@H](CC(C)C)C(=O)[C@@]3(C)CO3)CC2)cc1]

LC/MS calc'd for C61H77N9O11 (1111.6); found: 1112.3 (M+1).

### Example 1ab

### (1-((4-(3-(2,4-dihydroxy-5-isopropylphenyl)-5-(ethylcarbamoyl)-4H-1,2,4-triazol-4-yl)phenyl)sulfonyl)piperidin-4-yl)methyl ((S)-1-(((S)-4-methyl-1-(((S)-1-(((S)-4-methyl-1-((R)-2-methyloxiran-2-yl)-1-oxopentan-2-yl)amino)-1-oxo-3-phenylpropan-2-yl)amino)-1-oxopentan-2-yl)amino)-1-oxo-4-phenylbutan-2-yl)carbamate

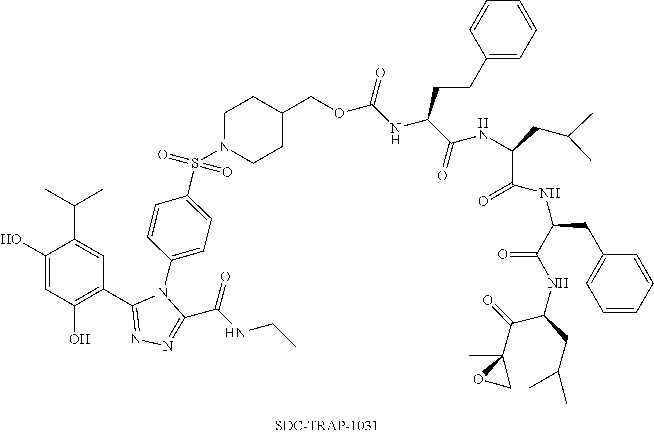 [SMILES: CCNC(=O)c1nnc(-c2cc(C(C)C)c(O)cc2O)n1-c1ccc(S(=O)(=O)N2CCC(COC(=O)N[C@@H](CCc3ccccc3)C(=O)N[C@@H](CC(C)C)C(=O)N[C@@H](Cc3ccccc3)C(=O)N[C@@H](CC(C)C)C(=O)[C@@]3(C)CO3)CC2)cc1]

LC/MS calc'd for C61H79N9O12S (1161.6); found: 1162.1 (M+1).

### Example 1ac

### (1-(4-(3-(2,4-dihydroxy-5-isopropylphenyl)-5-(ethylcarbamoyl)-4H-1,2,4-triazol-4-yl)benzoyl)piperidin-4-yl)methyl ((S)-1-(((S)-4-methyl-1-(((S)-1-(((S)-4-methyl-1-((R)-2-methyloxiran-2-yl)-1-oxopentan-2-yl)amino)-1-oxo-3-phenylpropan-2-yl)amino)-1-oxopentan-2-yl)amino)-1-oxo-4-phenylbutan-2-yl)carbamate

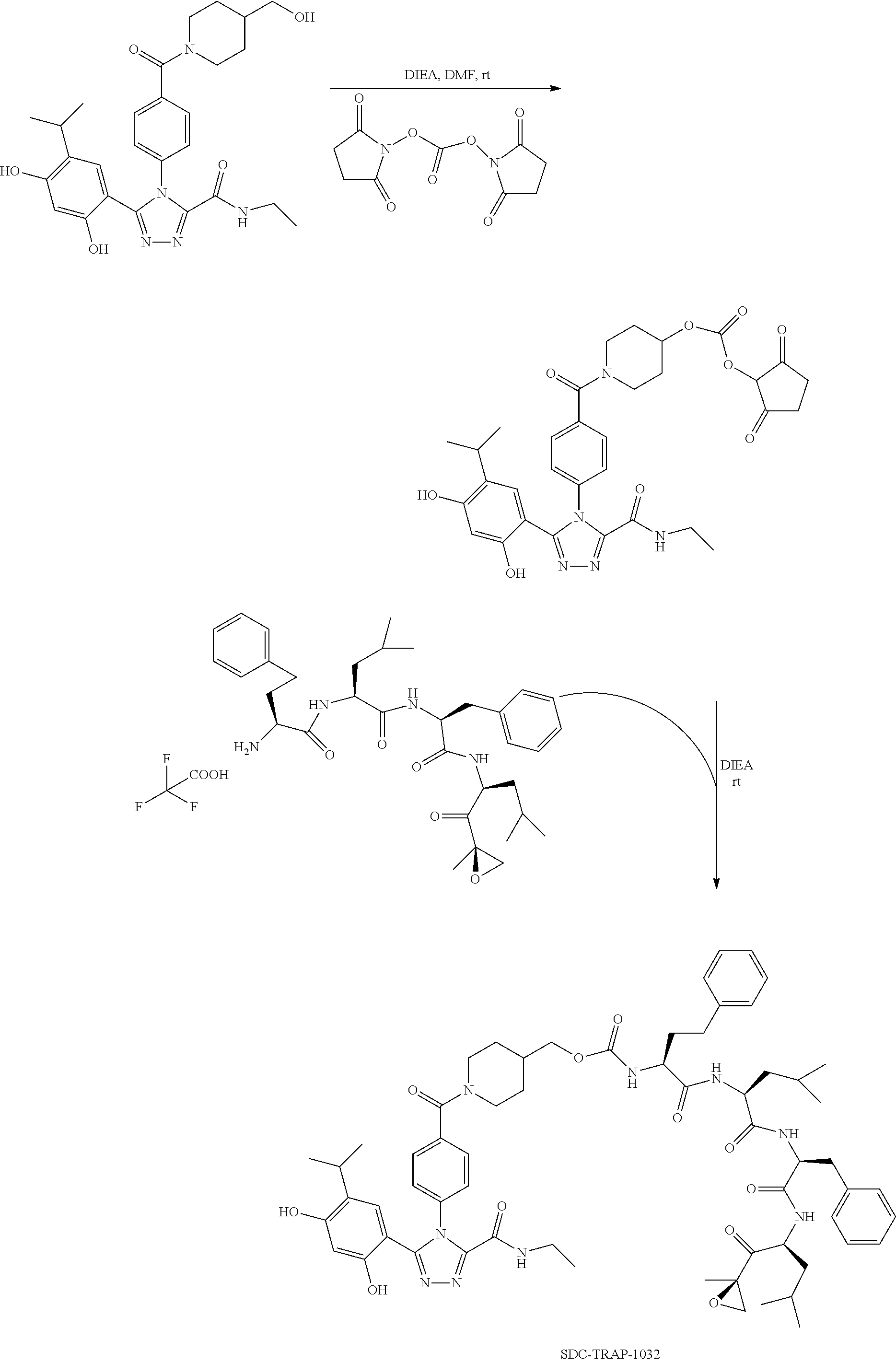 [SMILES: CCNC(=O)c1nnc(-c2cc(C(C)C)c(O)cc2O)n1-c1ccc(C(=O)N2CCC(CO)CC2)cc1 ; O=C(ON1C(=O)CCC1=O)ON1C(=O)CCC1=O ; CCNC(=O)c1nnc(-c2cc(C(C)C)c(O)cc2O)n1-c1ccc(C(=O)N2CCC(OC(=O)OC3C(=O)CCC3=O)CC2)cc1 ; O=C(O)C(F)(F)F ; CC(C)C[C@H](NC(=O)[C@@H](N)CCc1ccccc1)C(=O)N[C@@H](Cc1ccccc1)C(=O)N[C@@H](CC(C)C)C(=O)[C@@]1(C)CO1 ; CCNC(=O)c1nnc(-c2cc(C(C)C)c(O)cc2O)n1-c1ccc(C(=O)N2CCC(COC(=O)N[C@@H](CCc3ccccc3)C(=O)N[C@@H](CC(C)C)C(=O)N[C@@H](Cc3ccccc3)C(=O)N[C@@H](CC(C)C)C(=O)[C@@]3(C)CO3)CC2)cc1]

General procedure: To a stirred solution of 5-(2,4-dihydroxy-5-isopropylphenyl)-N-ethyl-4-(4-(4-(hydroxymethyl)piperidine-1-carbonyl)phenyl)-4H-1,2,4-triazole-3-carboxamide (38.3 mg, 0.075 mmol) and bis(2,5-dioxopyrrolidin-1-yl) carbonate (40 mg, 0.16 mmol) in DMF (1.5 mL) was added DIEA (0.3 mL). After 4 h, to the reaction mixture was added (1-(4-(3-(2,4-dihydroxy-5-isopropylphenyl)-5-(ethylcarbamoyl)-4H-1,2,4-triazol-4-yl)benzoyl)piperidin-4-yl)methyl ((S)-1-(((S)-4-methyl-1-(((S)-1-(((S)-4-methyl-1-((R)-2-methyloxiran-2-yl)-1-oxopentan-2-yl)amino)-1-oxo-3-phenylpropan-2-yl)amino)-1-oxopentan-2-yl)amino)-1-oxo-4-phenylbutan-2-yl)carbamate (63 mg, 0.089 mmol) in one portion followed by DIEA (0.3 mL). Stirring was then continued at room temperature overnight. The reaction was then quenched by adding a mixture of ice-H₂O (10 mL). Precipitated material was collected by filtration and purified by SGC on a ISCIO Combiflash system using a mixture of DCM and methanol as eluent to provide the final product, (1-(4-(3-(2,4-dihydroxy-5-isopropylphenyl)-5-(ethylcarbamoyl)-4H-1,2,4-triazol-4-yl)benzoyl)piperidin-4-yl)methyl ((S)-1-(((S)-4-methyl-1-(((S)-1-(((S)-4-methyl-1-((R)-2-methyloxiran-2-yl)-1-oxopentan-2-yl)amino)-1-oxo-3-phenylpropan-2-yl)amino)-1-oxopentan-2-yl)amino)-1-oxo-4-phenylbutan-2-yl)carbamate, as a white powder (19.5 mg, 23% yield). LC/MS calc'd for C62H79N9O11 (1125.6); found: 1126.2 (M+1).

### Example 1ad

### 1-(4-(3-(2,4-dihydroxy-5-isopropylphenyl)-5-(ethylcarbamoyl)-4H-1,2,4-triazol-4-yl)benzoyl)piperidin-4-yl)methyl 4-((4S,7S,10S,13S)-10-benzyl-7-isobutyl-15-methyl-13-((R)-2-methyloxirane-2-carbonyl)-2,5,8,11-tetraoxo-4-phenethyl-3,6,9,12-tetraazahexadecyl)piperazine-1-carboxylate

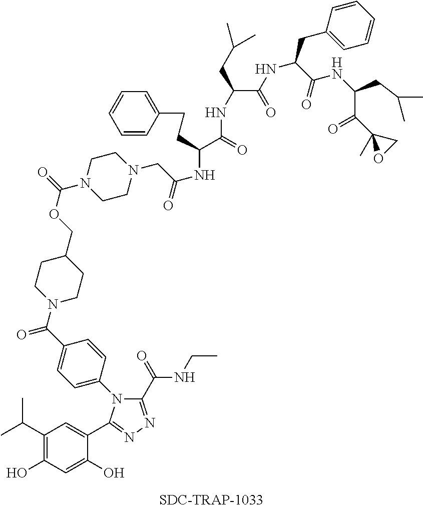 [SMILES: CCNC(=O)c1nnc(-c2cc(C(C)C)c(O)cc2O)n1-c1ccc(C(=O)N2CCC(COC(=O)N3CCN(CC(=O)N[C@@H](CCc4ccccc4)C(=O)N[C@@H](CC(C)C)C(=O)N[C@@H](Cc4ccccc4)C(=O)N[C@@H](CC(C)C)C(=O)[C@@]4(C)CO4)CC3)CC2)cc1]

LC/MS calc'd for C68H89N11O12 (1251.6); found: 1252.0 (M+1). ¹H NMR (400 MHz, Chloroform-d) δ 11.35 (br, 1H), 8.53 (br, 1H), 7.62 (d, J=8.1 Hz, 2H), 7.42 (m, 3H), 7.25-6.88 (m, 13H), 6.66 (d, J=8.8 Hz, 1H), 6.51 (d, J=13.0 Hz, 2H), 5.08-4.31 (m, 4H), 3.77-1.22 (m, 41H), 0.92-0.60 (m, 21H).

### Example 1ae

### (5S,8S,11S,14S)-11-benzyl-8-isobutyl-16-methyl-14-((R)-2-methyloxirane-2-carbonyl)-3,6,9,12-tetraoxo-5-phenethyl-4,7,10,13-tetraazaheptadecyl 4-(4-(3-(2,4-dihydroxy-5-isopropylphenyl)-5-hydroxy-4H-1,2,4-triazol-4-yl)-2-fluorobenzyl)piperazine-1-carboxylate

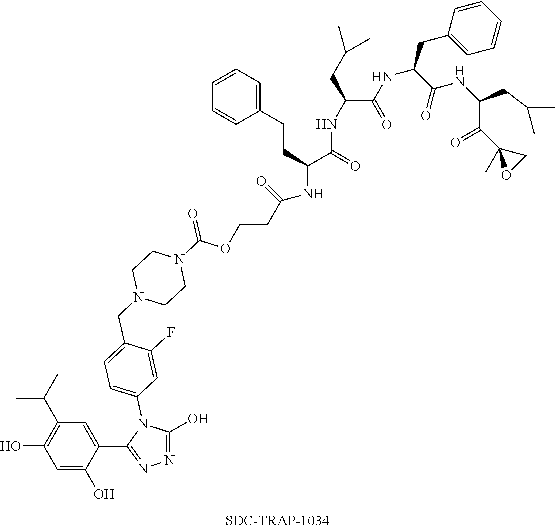 [SMILES: CC(C)C[C@H](NC(=O)[C@H](CCc1ccccc1)NC(=O)CCOC(=O)N1CCN(Cc2ccc(-n3c(O)nnc3-c3cc(C(C)C)c(O)cc3O)cc2F)CC1)C(=O)N[C@@H](Cc1ccccc1)C(=O)N[C@@H](CC(C)C)C(=O)[C@@]1(C)CO1]

LC/MS calc'd for C60H76FN9O11 (1117.6); found: 1118.2 (M+1). ¹H NMR (400 MHz, Chloroform-d) δ 7.78 (dd, J=8.0, 22.0 Hz, 1H), 7.63 (d, J=8.0 Hz, 1H), 7.50-7.38 (m, 3H), 7.35-7.01 (m, 14H), 6.58 (s, 1H), 6.39 (s, 1H), 5.31 (s, 1H), 4.68-4.47 (m, 2H), 4.45-4.29 (m, 4H), 3.60 (s, 2H), 3.48-2.33 (m, 21H), 1.92 (ddd, J=48.4, 13.9, 6.7 Hz, 2H), 1.51-1.37 (m, 4H), 1.26 (ddd, J=14.2, 10.6, 4.2 Hz, 1H), 0.91-0.76 (m, 18H).

### Example 2

This example illustrates how a HER2 degradation assay may be used as a test to determine and select Hsp90-targeting moieties suitable for use in SDC-TRAPs of the invention, and further illustrates the ability of SDC-TRAPs to target cells preferentially expressing Hsp90. Such a test may further be used to determine the Hsp90 binding ability of SDC-TRAPs of the invention, as well as through competitive binding assays and cell-based Hsp90 client protein degradation assays known in the aroom temperature.

Degradation of HER2 in Cells after Treatment with an SDC-TRAP of the Invention

Method 1:

BT-474 cells are treated with 0.5 μM, 2 μM, or 5 μM of 17-AAG (a positive control) or 0.5 μM, 2 μM, or 5 μM of an Hsp90-targeting moiety or conjugate of the invention overnight in DMEM medium. After treatment, each cytoplasmic sample is prepared from 1×10⁶ cells by incubation of cell lysis buffer (#9803, Cell Signaling Technology) on ice for 10 minutes. The resulting supernatant used as the cytosol fractions is dissolved with sample buffer for SDS-PAGE and run on a SDS-PAGE gel, blotted onto a nitrocellulose membrane by using semi-dry transfer. Non-specific binding to nitrocellulose is blocked with 5% skim milk in TBS with 0.5% Tween at room temperature for 1 hour, then probed with anti-HER2/ErB2 mAb (rabbit IgG, #2242, Cell Signaling) and anti-Tubulin (T9026, Sigma) as housekeeping control protein. HRP-conjugated goat anti-rabbit IgG (H+L) and HRP-conjugated horse anti-mouse IgG (H+L) are used as secondary Ab (#7074, #7076, Cell Signaling) and LumiGLO reagent, 20× Peroxide (#7003, Cell Signaling) is used for visualization. The Hsp90 client protein HER2 is degraded when cells are treated with Hsp90-targeting moieties or SDC-TRAPs of the invention. 0.5 μM of 17-AAG, a known Hsp90 inhibitor used as a positive control, causes partial degradation of HER2.

Method 2:

BT-474 cells are plated in the interior 60 wells of a 96 well black clear bottom plate (20,000 cells/well) in DMEM medium, with DMEM media in the surrounding 36 wells, and incubated at 37° C. with 5% CO₂ overnight. On the second day, concentration response curve source plates are produced (10 point, 3-fold dilution of compounds in DMSO) followed by a 1:30 dilution in an intermediate dilution plate containing DMEM. Compound is transferred from the intermediate plate to the cell plate at a dilution of 1:10. The cells are then incubated at 37° C. with 5% CO₂ for 24 hours.

Cells are then fixed in 4% phosphate-buffered paraformaldehyde for 30 minutes at room temperature and then permeabilized by washing five times with 0.1% Triton X-100 in PBS for 5 minutes at room temperature on a shaker. Cells are blocked with Odyssey Blocking Buffer (LI-COR, #927-40000) on a shaker at room temperature for 1.5 hours, followed by incubation with HER2 antibody (CST, #2165) diluted 1:400 in blocking buffer overnight on a shaker at 4° C. Cells are washed five times with 0.1% Tween-20 in PBS for 5 minutes at room temperature on a shaker and incubated with fluorescently-labeled secondary antibody (LI-COR, #926-32211) diluted 1:1000 in blocking buffer, and DRAQ5 nuclear stain (Biostatus Limited, #DRAQ5) diluted 1:10,000, at room temperature on a shaker for 1 hour. Cells are washed 5 times with 0.1% Tween-20 in PBS for 5 minutes at room temperature on a shaker and imaged on a LI-COR Odyssey imaging station. The raw data is normalized to DRAQ5 and the HER2 EC₅₀ is calculated using XLfit™.

The above procedures are utilized to generate HER2 degradation data, which show the ability of SDC-TRAPs to target cells preferentially expressing Hsp90.

### Example 3

This example illustrates a method of assessing the cytotoxicity of SDC-TRAPs.

Cell Lines.

Human H3122 NSCLC cells are obtained and grown in RPMI in the presence of fetal bovine serum (10%), 2 mM L-glutamine and antibiotics (100 IU/ml penicillin and 100 μg/ml streptomycin, Sigma Aldrich.) Cells are maintained at 37° C., 5% CO₂ atmosphere.

Cell Viability Assays.

Cell viability is measured using the CellTiter-Glo® assay (Promega). In brief, cells are plated in 96-well plates in triplicate at optimal seeding density (determined empirically) and incubated at 37° C., 5% CO₂ atmosphere for 24 hr prior to the addition of drug or vehicle (0.3% DMSO) to the culture medium. At the end of the assay, CellTiter-Glo is added to the wells per manufacturer's recommendation, shaken for two minutes and incubated for 10 minutes at room temperature. Luminescence (0.1 sec) is measured with a Victor II microplate reader (Perkin Elmer) and the resulting data is used to calculate cell viability, normalized to vehicle control.

All publications, patent applications, patents, and other documents cited herein are incorporated by reference in their entirety. In case of conflict, the present specification, including definitions, will control.

The specification should be understood as disclosing and encompassing all possible permutations and combinations of the described aspects, embodiments, and examples unless the context indicates otherwise. One of ordinary skill in the art will appreciate that the invention can be practiced by other than the summarized and described aspect, embodiments, and examples, which are presented for purposes of illustration.

1.-20. (canceled)

21. A SDC-TRAP comprising an Hsp90 binding moiety and a peptide epoxy ketone protease inhibitor.

22. The SDC-TRAP of claim 21, wherein the Hsp90 binding moiety interacts with a protein that is overexpressed in a target cell compared to a normal cell.

23. The SDC-TRAP of claim 22, wherein the target cell is a cancer cell.

24. The SDC-TRAP of claim 21, wherein the Hsp90 binding moiety is an Hsp90 ligand or a prodrug thereof.

25. The SDC-TRAP of claim 24, wherein the Hsp90 ligand is an Hsp90 inhibitor.

26. The SDC-TRAP of claim 25, wherein the Hsp90 inhibitor is selected from the group consisting of ganetespib, geldanamycins, macbecins, tripterins, tanespimycins, and radicicols.

27. The SDC-TRAP of claim 21, wherein the SDC-TRAP is selected from the group consisting of: SDC-TRAP-1001, SDC-TRAP-1002, SDC-TRAP-1003, SDC-TRAP-1004, SDC-TRAP-1005, SDC-TRAP-1006, SDC-TRAP-1007, SDC-TRAP-1008, SDC-TRAP-1009, SDC-TRAP-1010, SDC-TRAP-1011, SDC-TRAP-1012, SDC-TRAP-1013, SDC-TRAP-1014, SDC-TRAP-1015, SDC-TRAP-1016, SDC-TRAP-1017, SDC-TRAP-1018, SDC-TRAP-1019, SDC-TRAP-1020, SDC-TRAP-1021, SDC-TRAP-1022, SDC-TRAP-1023, SDC-TRAP-1024, SDC-TRAP-1025, SDC-TRAP-1026, SDC-TRAP-10127, SDC-TRAP-1028, SDC-TRAP-1029, SDC-TRAP-1030, SDC-TRAP-1031, SDC-TRAP-1032, SDC-TRAP-1033 and SDC-TRAP-1034.

28. The SDC-TRAP of claim 21, wherein the SDC-TRAP exhibits decreased toxicity and increased efficacy compared to the Hsp90 binding moiety or peptide epoxy ketone protease inhibitor used alone.

29. The SDC-TRAP of claim 22, wherein the SDC-TRAP is present in a target cell for at least 24 hours.

30. The SDC-TRAP of claim 22, wherein the SDC-TRAP is released in a target cell for at least 6 hours.

31. The SDC-TRAP of claim 21, wherein the Hsp90 binding moiety and the peptide epoxy ketone protease inhibitor are covalently attached.

32. The SDC-TRAP of claim 31, wherein the Hsp90 binding moiety and the peptide epoxy ketone protease inhibitor are covalently attached by a linker.

33. The SDC-TRAP of claim 32, wherein the linker comprises a cleavable linker.

34. The SDC-TRAP of claim 33, wherein the cleavable linker comprises an enzymatically cleavable linker.

35. The SDC-TRAP of claim 32, wherein the linker is selected from the group consisting of disulfide, carbamate, amide, ester, and ether linkers.

36. The SDC-TRAP of claim 21, wherein the SDC-TRAP is able to enter a cell by passive diffusion or active transport.

37. The SDC-TRAP of claim 21, wherein the peptide epoxy ketone protease inhibitor is carfilzomib or its fragment or analog.

38.-55. (canceled)

56. A pharmaceutical composition comprising a therapeutically effective amount of at least one SDC-TRAP, and at least one pharmaceutical excipient, wherein the SDC-TRAP comprises the SDC-TRAP of claim 21.

57.-58. (canceled)

59. The pharmaceutical composition of claim 56, wherein the SDC-TRAP is selected from the group consisting of: SDC-TRAP-1001, SDC-TRAP-1002, SDC-TRAP-1003, SDC-TRAP-1004, SDC-TRAP-1005, SDC-TRAP-1006, SDC-TRAP-1007, SDC-TRAP-1008, SDC-TRAP-1009, SDC-TRAP-1010, SDC-TRAP-1011, SDC-TRAP-1012, SDC-TRAP-1013, SDC-TRAP-1014, SDC-TRAP-1015, SDC-TRAP-1016, SDC-TRAP-1017, SDC-TRAP-1018, SDC-TRAP-1019, SDC-TRAP-1020, SDC-TRAP-1021, SDC-TRAP-1022, SDC-TRAP-1023, SDC-TRAP-1024, SDC-TRAP-1025, SDC-TRAP-1026, SDC-TRAP-10127, SDC-TRAP-1028, SDC-TRAP-1029, SDC-TRAP-1030, SDC-TRAP-1031, SDC-TRAP-1032, SDC-TRAP-1033 and SDC-TRAP-1034.

60. A method for treating a subject in need thereof comprising administering a therapeutically effective amount of at least one SDC-TRAP to the subject, thereby treating the subject, wherein the SDC-TRAP comprises the SDC-TRAP of claim 21.

61. (canceled)

62. The method of claim 60, wherein the subject is suffering from a disease of disorder selected from the group consisting of: cancer, autoimmune disease, graft or transplant-related condition, neurodegenerative disease, fibrotic-associated condition, ischemic-related conditions, infection (viral, parasitic or prokaryotic) and diseases associated with bone loss.

63. The method of claim 62, wherein the subject is suffering from cancer.

64.-67. (canceled)

68. A kit for imaging, diagnosing, and/or selecting a subject comprising at least one SDC-TRAP and instructions for administering an effective amount of the at least one SDC-TRAP to the subject, thereby imaging, diagnosing, and/or selecting the subject, wherein the SDC-TRAP comprises the SDC-TRAP of claim 21.

69.-71. (canceled)
